(19) United States
(12) Patent Application Publication
(10) Pub. No.: US 2023/0407298 A1
(43) Pub. Date: Dec. 21, 2023

(54) METHODS FOR INHIBITING CHMP7 EXPRESSION IN NEURONAL CELLS FOR THE TREATMENT OF NEURODEGENERATIVE DISORDERS

(71) Applicant: THE JOHNS HOPKINS UNIVERSITY, Baltimore, MD (US)

(72) Inventors: Jeffrey D. Rothstein, Baltimore, MD (US); Alyssa Coyne, Baltimore, MD (US)

(21) Appl. No.: 18/036,022

(22) PCT Filed: Nov. 10, 2021
(86) PCT No.: PCT/US2021/058780

§ 371 Date: May 9, 2023

Related U.S. Application Data
(60) Provisional application No. 63/111,882, filed on Nov. 10, 2020.

(51) Int. Cl. C12N 15/113; G01N 33/50; A61P 25/28

(52) U.S. Cl. CPC C12N 15/113; G01N 33/5058; A61P 25/28; C12N 2310/11

(57) ABSTRACT

The present disclosure relates to methods of screening compounds which reduce or inhibit CHMP7 in neuronal cells or a population of cells and methods of inhibiting CHMP7 expression in a cell to treat one or more subjects suffering from one or more neurodegenerative diseases.

### RELATED APPLICATION INFORMATION

This application claims priority to U.S. Application No. 63/111,882 filed on Nov. 10, 2020, the contents of which is herein incorporated by reference.

### STATEMENT OF GOVERNMENTAL INTEREST

This invention was made with government support under grant AG062171 awarded by the National Institutes of Health. The government has certain rights in the invention.

### INCORPORATION-BY-REFERENCE OF MATERIAL SUBMITTED ELECTRONICALLY

The instant application contains a Sequence Listing which has been submitted in ASCII format via EFS-Web and is hereby incorporated by reference in its entirety. Said ASCII copy, created on Nov. 10, 2021, is named 38922-601_ST25.txt and is 4,824 bytes in size.

### BACKGROUND

The motor neuron disease Amyotrophic Lateral Sclerosis (ALS) is a devastating neurodegenerative disease affecting multiple neuronal and glial subtypes within the motor cortex and spinal cord. Approximately 10% of ALS cases are familial (fALS) with more than 20 familial mutations identified to date. The most common genetic cause of ALS is a GGGGCC (G₄C₂) (SEQ ID NO: 1) hexanucleotide repeat expansion (HRE) in the C9orf72 gene, accounting for about 40% of familial ALS and 8% of sporadic ALS. However, 90% of ALS cases are sporadic (sALS) with no known underlying causative genetic mutation. Despite the heterogenous nature of ALS etiology, the nuclear clearing and cytoplasmic mislocalization and/or aggregates of TAR DNA Binding Protein (TDP-43), has emerged as a prominent pathological hallmark of endstage familial and sporadic disease (36-38).

While studies of genetic forms of ALS including C9orf72 have provided novel insights into underlying disease mechanisms and potential therapeutic strategies, these discoveries do not necessarily translate to sALS. The lack of preclinical animal model systems that faithfully reproduce the heterogenous nature of humans ALS highlights a major challenge in understanding 90% of ALS pathophysiology. However, the reprogramming of patient derived fibroblasts and peripheral blood mononuclear cells (PBMCs) into induced pluripotent stem cells (iPSCs) and then differentiated into neuronal and glial cells are being employed to model human disease (39-40). Studies have shown that C9orf72 iPSC derived motor neurons (iPSNs) recapitulate key pathological features of disease seen in postmortem human nervous system (41-42). Collectively, iPSNs provide a useful and relevant platform for studying mechanisms underlying ALS and other neurodegenerative diseases.

Molecular changes to the composition of the nuclear pore complex (NPC) have been identified as an early and key pathomechanism in C9orf72 disease (43). The NPC is comprised of multiple copies of ˜30 distinct Nups and can be classified as either: scaffold Nups and mobile Nups. Scaffold Nups build the 8-fold radial architecture of the NPC and secure it to the nuclear envelope. The NPC scaffold provides anchor points for FG-Nups which fill the central transport channel and mediate the nucleocytoplasmic exchange of macromolecules. Some Nups can also impact gene transcription (44-47). Given the critical roles of the NPC in maintaining overall cellular function, alterations in specific Nups may affect neuronal viability and subsequently give rise to disease pathology.

A subset of 8 Nups are significantly reduced within the NPC and nucleoplasm of C9orf72 and isolated postmortem nuclei. Moreover, steady state Nup mRNA levels, and Nup mRNA stability and association with actively translating polyribosome fractions are not altered in C9orf72 iPSNs (43). Collectively, these data suggest that early alterations in Nup protein homeostasis may underlie reduction of Nups and functional transport in C9orf72 ALS/FTD pathogenesis. However, the molecular pathway that underlies neuronal NPC disruption and whether this pathophysiology is more broadly applicable to sporadic disease remains unknown.

Work in several model systems including yeast supports the existence of molecular pathways that can impact Nup and NPC homeostasis within the nuclear envelope and nucleoplasm. The recruitment of the endosomal sorting complexes required for transport (ESCRT) machinery by LEM family inner nuclear membrane (INM) proteins has been implicated in the proteasomal degradation of misassembled Nups (48) and the sealing of defective NPCs (49-50). Analogous mechanisms likely function in non-neuronal mammalian cells to facilitate the removal of Nups and NPCs (51). Recent work in yeast reveals that both individual Nups and NPCs can also be targets of autophagy (52-53). Critically, the nuclear recruitment and/or retention of CHMP7 appears to be an initiating step in proper Nup and NPC homeostasis (49, 50, 54), although little is known regarding CHMP7 mediated Nup proteostasis in human neurons and altered Nup levels in neurodegeneration.

### SUMMARY

Using induced pluripotent stem cell (iPSC) derived spinal neurons (iPSNs) and postmortem human tissue the inventors now show that the nuclear envelope-specific ESCRT protein CHMP7 and its downstream effecter AAA-ATPase VPS4, are increased in C9orf72 and sALS nuclei. In accordance with the inventors' previous study suggesting that G₄C₂ repeat RNA initiates the reduction of key Nups, overexpression of G₄C₂ repeat RNA alone results in a nuclear increase in CHMP7 and VPS4 in iPSNs.

Importantly, using Trim Away (25) and antisense oligonucleotides (ASOs), the inventors now provide multiple lines of evidence that knockdown of CHMP7 mitigates the reduction of specific Nups from the nucleoplasm and NPCs in C9orf72 iPSNs. Additionally, CHMP7 ASOs mitigate defects in stathmin-2 splicing and alleviate downstream neuronal toxicity. Mechanistically, impaired nuclear export, but not increased nuclear recruitment, of CHMP7 leads to reduced expression of specific Nups and human neurons. Furthermore, impaired nuclear export of CHMP7 may be the result of G₄C2 mediated reduction of the presence of CHMP7 in XPO1 complexes within neuronal nuclei. Additionally, CHMP7 mediated reduction in specific Nups results in alterations in stathmin-2 splicing and TDP-43 mislocalization. Collectively, these data show that in human neurons, aberrant activation of the ESCRT-III pathway as a result of pathologic G4C2 repeat RNA is a substantial and early contributor to Nup alterations in ALS. Additionally, these findings highlight the potential for CHMP7 as a therapeutic target in familial and sporadic ALS/FTD and related neurodegenerative diseases characterized by Nup reduction and TDP-43 pathology.

Thus, one embodiment, the present disclosure provides methods for screening compounds which reduce or inhibit CHMP7 in a cell or population of cells, such as a neuronal cell or population of neuronal cells. The methods comprise administering to a cell or population of cells (such as a neuronal cell or population of neuronal cells) at least one compound and determining if the compound reduces or inhibits CHMP7 expression in the cell or population of cells. Compounds that reduce or inhibit CHMP7 expression are then selected. Compounds that can screened and then selected pursuant to the above methods can include one or more of a small molecule or a pharmaceutically acceptable salt thereof, an antibody (such as a monoclonal antibody, a chimeric antibody, a humanized antibody, a fully human antibody, etc.) or antigen-binding fragment thereof, an oligonucleotide (e.g., antisense oligonucleotide or a pharmaceutically acceptable salt thereof), a small-interfering RNA (siRNA), a microRNA (miRNA), a peptide, a peptidomimetic, or any combinations thereof.

In another embodiment, the present disclosure provides methods of inhibiting CHMP7 expression in a cell or population of cells (such as a neuronal cell or population of neuronal cells). The methods comprise administering to the cells an effective amount of a CHMP7 inhibiting agent. In this aspect, the CHMP7 inhibiting agent can be one or more of a small molecule or a pharmaceutically acceptable salt thereof, an antibody (such as a monoclonal antibody, a chimeric antibody, a humanized antibody, a fully human antibody, etc.) or antigen-binding fragment thereof, an oligonucleotide (e.g., antisense oligonucleotide or a pharmaceutically acceptable salt thereof), a small-interfering RNA (siRNA), a microRNA (miRNA), a peptide, a peptidomimetic, or any combinations thereof. In yet further aspects, the CHMP7 inhibiting agent is one or more antisense oligonucleotides or pharmaceutically acceptable salts thereof.

In yet another embodiment, the present disclosure provides methods of treating one or more neurodegenerative diseases in a subject in need thereof. The methods comprise administering to the subject in need of treatment thereof an effective or therapeutically effective amount of at least one CHMP7 inhibiting agent. In this aspect, the CHMP7 inhibiting agent can be one or more of a small molecule or a pharmaceutically acceptable salt thereof, an antibody (such as a monoclonal antibody, a chimeric antibody, a humanized antibody, a fully human antibody, etc.) or antigen-binding fragment thereof, an oligonucleotide (e.g., antisense oligonucleotide or a pharmaceutically acceptable salt thereof), a small-interfering RNA (siRNA), a microRNA (miRNA), a peptide, a peptidomimetic, or any combinations thereof. In yet other aspects, the CHMP7 inhibiting agent is one or more antisense oligonucleotides or pharmaceutically acceptable salts thereof. In yet other further aspects of this method, the subject to be treated is a human. In still yet other aspects, the CHMP7 inhibiting agent is administered to the subject in an amount of about 0.001 mg/kg to about 1000 mg/kg. In yet further aspects, the neurodegenerative disease being treated is Amyotrophic Lateral Sclerosis (ALS), Huntington's disease, Alzheimer's disease, Parkinson's disease, Lewy body dementia, multiple sclerosis, or frontotemporal degeneration (FTD). In still further aspects, the neurodegenerative disease is ALS or FTD.

### BRIEF DESCRIPTION OF THE DRAWINGS

FIG. 1 illustrates that the nuclear expression of CHMP7 and VPS4 is increased in C9orf72 iPSN and postmortem nuclei. (A) Maximum intensity projections from SIM imaging of CHMP7 and VPS4 in nuclei isolated from control and C9orf72 iPSNs. Genotype as indicated on left, antibody and time point as indicated on top (FIGS. 1B-1C). Quantification of CHMP7 (FIG. 1B) and VPS4 (FIG. 1C) spots. n=8 control and 8 C9orf72 iPSC lines (including 1 isogenic pair), 50 NeuN+ nuclei per line/time point. Two-way ANOVA with Tukey's multiple comparison test was used to calculate statistical significance. ** p<0.01, **** p<0.0001 (FIGS. 1D-1E). Western blot (FIG. 1D) and quantification (FIG. 1E) for CHMP7 and VPS4 levels in nuclei isolated from day 32 control and C9orf72 iPSNs. Antibodies as indicated on right, genotype as indicated on bottom. n=4 control and 4 C9orf72 iPSC lines. Student's t-test was used to calculate statistical significance. * p<0.05. (FIG. 1F) Maximum intensity projections from SIM imaging of CHMP7 and VPS4 in nuclei isolated from control and C9orf72 postmortem tissue. Genotype as indicated on left, antibody and CNS region as indicated on top. (FIGS. 1G-1H) Quantification of CHMP7 (FIG. 1G) and VPS4 (FIG. 1H) spots. n=3 control and 3 C9orf72 cases, 50 NeuN+ nuclei per case/CNS region. Two-way ANOVA with Tukey's multiple comparison test was used to calculate statistical significance. **** p<0.0001. Scale bar=5 μm.

FIG. 2 illustrates that the nuclear localization and expression of CHMP7 is increased in thin sections from postmortem C9orf72 patient motor cortex (FIG. 2A). Immunostaining for CHMP7 in paraffin embedded postmortem motor and occipital cortex. Genotype as indicated on left, brain region and antibody as indicated on top (FIG. 2B). Quantification of nuclear/cytoplasmic ratio of CHMP7 immunostaining. n=7 control and 7 C9orf72 cases, at least 50 Map2+ cells per case and brain region. Student's t-test was used to calculate statistical significance. **** p<0.0001. Scale bar=10 μm.

FIG. 3 illustrates that knockdown of CHMP7 restores the nuclear expression of specific Nups in C9orf72 iPSNs (A-C). Western blot (FIG. 3A) and quantification (FIGS. 3B-3C) of Trim21 GFP mediated reduction in CHMP7 in control and C9orf72 iPSNs. Student's t-test was used to calculate statistical significance. * p<0.05, ** p<0.01. (FIG. 3D) Maximum intensity projections from SIM imaging of nuclei isolated from control and C9orf72 iPSNs following knockdown of CHMP7. Antibody used for Trim21 GFP mediated knockdown as indicated on left, genotype and antibodies as indicated on top (FIGS. 3E-3J). Quantification of spots and volume. n=3 control and 3 C9orf72 iPSC lines, 50 GFP+ nuclei per line/knockdown. Two-way ANOVA with Tukey's multiple comparison test was used to calculate statistical significance. **** p<0.0001. Scale bar=5 μm.

FIG. 4 illustrates that ASO mediated knockdown of CHMP7 restores the nuclear expression of specific Nups and mitigates glutamate induced excitotoxicity in C9orf72 IPSNs (FIG. 4A). Maximum intensity projections from SIM imaging of nuclei isolated from control and C9orf72 iPSNs following 2-week exposure to 5 μM scrambled control ASO or CHMP7 ASO 2. Treatment as indicated on left, genotype and antibodies as indicated on top (FIGS. 4B-4G). Quantification of spots and volume. n=4 control and 4 C9orf72 iPSC lines, 50 NeuN+ nuclei per line/treatment. Two-way ANOVA with Tukey's multiple comparison test was used to calculate statistical significance. **** p<0.0001 (FIG. 4H). Quantification of percent cell death as measured by propidium iodide (PI) incorporation following exposure to glutamate in control and C9orf72 iPSNs following 2-week exposure to 5 μM scrambled control ASO or CHMP7 ASO 2. n=4 control and 4 C9orf72 iPSC lines, 5 frames per well. Two-way ANOVA with Tukey's multiple comparison test was used to calculate statistical significance. **** p<0.0001. Scale bar=5 μm.

FIG. 5 illustrates that knockdown of LEMD2 does not restore the nuclear expression of specific Nups in C9orf72 iPSNs (FIG. 5A-5C). Western blot (FIG. 5A) and quantification (FIG. 5B-5C) of Trim21 GFP mediated reduction in LEMD2 in control and C9orf72 iPSNs. Student's t-test was used to calculate statistical significance. * p<0.05. (FIG. 5D) Maximum intensity projections from SIM imaging of nuclei isolated from control and C9orf72 iPSNs following knockdown of LEMD2. Antibody used for Trim21 GFP mediated knockdown as indicated on left, genotype and antibodies as indicated on top (FIGS. 5E-5K). Quantification of spots and volume. n=3 control and 3 C9orf72 iPSC lines, 50 GFP+ nuclei per line/knockdown. Two-way ANOVA with Tukey's multiple comparison test was used to calculate statistical significance. **** p<0.0001. Scale bar=5 μm.

FIG. 6 illustrates that inhibition of CHMP7 nuclear export in wildtype iPSNs recapitulates C9orf72 mediated Nup alterations (FIG. 6A). Maximum intensity projections from SIM imaging of nuclei isolated from control iPSNs following Leptomycin B treatment. Treatment as indicated on left, antibodies as indicated on top. LMB=Leptomycin B. (FIG. 6B) Quantification of spots and volume. n=3 control iPSC lines, 50 NeuN+ nuclei per line/treatment. Student's t-test was used to calculate statistical significance. * p<0.05, **** p<0.0001 (FIG. 6C). Maximum intensity projections from SIM imaging of nuclei isolated from control iPSNs overexpressing GFP tagged CHMP7 variants. Overexpression as indicated on left, antibodies as indicated on top (FIG. 6D-6I). Quantification of spots and volume. n=4 control iPSC lines, 50 GFP+ nuclei per line/overexpression. One-way ANOVA with Tukey's multiple comparison test was used to calculate statistical significance. **** p<0.0001. Scale bar=5 μm.

FIG. 7 illustrates that G₄C₂ repeat RNA decreases the presence of CHMP7 in XPO1 complexes (FIG. 7A). Western blot following XPO1 immunoprecipitation. Genotype and treatment as indicated on top, antibody for immunoprecipitation as indicated on bottom, antibody for western blot as indicated on right (FIGS. 7B-7F). Quantification of CHMP7 (FIG. 7B), NXF3 (FIG. 7C), HuR (FIG. 7D), DDX3X (FIG. 7E), and RanBP1 (FIG. 7F), enrichment in XPO1 IP normalized to input and relative to XPO1 enrichment. n=4 control and 4 C9orf72 iPSC lines. One-way ANOVA with Tukey's multiple comparison test was used to calculate statistical significance. *** p<0.001, **** p<0.0001. (G) qRT-PCR for indicated RNAs following XPO1 immunoprecipitation. n=4 control and 4 C9orf72 iPSC lines.

FIG. 8 illustrates that nuclear expression of CHMP7 and VPS4 is increased as a result of G₄C₂ repeat RNA expression in iPSNs (FIG. 8A). Maximum intensity projections from SIM imaging of CHMP7 and VPS4 in nuclei isolated from control iPSNs overexpressing G₄C₂ repeat RNA only. Antibody as indicated on left, overexpression as indicated on top (FIGS. 8B-8E). Quantification (FIGS. 8B-8C) and histogram distribution (FIGS. 8D-8E) of CHMP7 (FIG. 8B, FIG. 8D) and VPS4 (FIG. 8C, FIG. 8E) spots. n=4 control iPSC lines, 100 NeuN+ nuclei per line/overexpression. One-way ANOVA with Tukey's multiple comparison test was used to calculate statistical significance. **** p<Scale bar=5 μm.

FIG. 9 illustrates that a dominant negative VPS4 partially mitigates alterations in nuclear POM121 expression (FIG. 9A). Maximum intensity projections from SIM imaging of CHMP7, VPS4, and POM121 in nuclei isolated from control and C9orf72 iPSNs overexpressing GFP tagged VPS4 variants. Overexpression as indicated on left, genotype and antibody as indicated on top (FIGS. 9B-9D). Quantification of CHMP7 (FIG. 9B), VPS4 (FIG. 9C), and POM121 (FIG. 9D) spots. n=4 control and 4 C9orf72 iPSC lines, 50 GFP+ nuclei per line/overexpression. Two-way ANOVA with Tukey's multiple comparison test was used to calculate statistical significance. * p<0.05, **** p<0.0001. Scale bar=5 μm.

FIG. 10 illustrates that CHMP7 ASOs reduce CHMP7 protein in iPSNs in a dose dependent manner (FIGS. 10A-10C). Western blot (FIG. 10A) and quantification (FIGS. for CHMP7 protein in control (10A-10B) and C9orf72 (FIG. 10A, FIG. 10C) iPSNs following 2-week exposure to scrambled control or CHMP7 targeting ASOs. Genotype as indicated on top, concentration and ASO as indicated on bottom. n=4 control and 4 C9orf72 IPSC lines. Two-way ANOVA with Tukey's multiple comparison test was used to calculate statistical significance. * p<0.05, *** p<0.001, **** p<0.0001.

FIG. 11 illustrates that Trim21 mediated knockdown of XPO1 in wildtype iPSNs recapitulates C9orf72 mediated alterations in the nuclear expression of CHMP7, VPS4, and specific Nups (FIGS. 11A-11B). Western blot (FIG. 11A) and quantification (FIG. 11B) of Trim21 GFP mediated reduction in XPO1 in wildtype iPSNs. n=3 control iPSC lines. Student's t-test was used to calculate statistical significance. * p<0.05. (FIG. 11C) Maximum intensity projections from SIM imaging of nuclei isolated from control iPSNs following Trim21 GFP mediated knockdown of XPO1. Antibody for knockdown as indicated on left, antibodies as indicated on top (FIG. 11D). Quantification of spots and volume. n=3 control iPSC lines, 50 GFP+ nuclei per line/knockdown. Student's t-test was used to calculate statistical significance. **** p<0.0001. Scale bar=5 μm.

FIG. 12 illustrates that leptomycin B treatment retains G₄C₂ repeat RNA in the nucleus of C9orf72 iPSNs (FIG. 12A). Western blot following nuclear/cytoplasmic fractionation of C9orf72 iPSNs. Fraction and treatment as indicated on bottom, antibody as indicated on right. In=input, Nuc=nucleus, Cyto=cytoplasm, LMB=Leptomycin B. (FIGS. 12B-12C) Quantification of RanBP1 (FIG. 12B) and DDX3X (FIG. 12C) protein enrichment in nuclear and cytoplasmic fractions following Leptomycin B treatment. n=3 C9orf72 iPSC lines. Two-way ANOVA with Tukey's multiple comparison test was used to calculate statistical significance. * p<0.05 (FIGS. 12D-12F). qRT-PCR for G₄C₂ repeat RNA (FIG. 12D), G₂C₄ repeat RNA (FIG. 12E), and C9orf72 RNA (FIG. 12F) following nuclear/cytoplasmic fractionation. n=3 C9orf72 iPSC lines.

FIG. 13 illustrates that G₄C₂ repeat RNA is not present in a complex with NXF1. (FIG. 13A) Western blot following NXF1 immunoprecipitation. Antibody for immunoprecipitation as indicated on bottom, antibody for western blot as indicated on right. In=input (FIG. 13B). qRT-PCR for indicated RNAs following NXF1 immunoprecipitation. n=4 control and 4 C9orf72 iPSC lines.

FIG. 14 illustrates the model for CHMP7 mediated Nup alterations in C9orf72 ALS/FTD. Expanded C9orf72 repeat RNA interferes with the association between XPO1 and CHMP7, but not other NES bearing cargo proteins, leading to increased nuclear CHMP7 levels. CHMP7 recruits VPS4 which subsequently leads to reduced nucleoporin levels in C9orf72 neuronal nuclei and NPCs.

FIG. 15 illustrates that a reduction in specific nucleoporins correlates with increased nuclear expression of CHMP7 in C9orf72 and sALS iPSN nuclei. (FIG. 15A) Maximum intensity projections from SIM imaging of Nups in nuclei isolated from control and sALS iPSNs at day 32 of differentiation. Genotype as indicated on left, antibody as indicated on top (FIG. 15B). Quantification of Nup spots. n=10 control and 17 sALS iPSC lines, 50 NeuN+ nuclei per line. Student's t-test was used to calculate statistical significance. **** p<0.0001 (FIG. 15C). Maximum intensity projections from SIM imaging of CHMP7 in nuclei isolated from control, C9orf72, and sALS iPSNs. Time point as indicated on left, genotype as indicated on top (FIG. 15D). Quantification of CHMP7 spots. n=10 control, 8 C9orf72, and 17 sALS iPSC lines, 50 NeuN+ nuclei per line. One-way ANOVA Tukey's multiple comparison test was used to calculate statistical significance. **** p<0.0001. (e-g) Western blot (FIG. 15E) and quantification (FIG. 15F-15G) for CHMP7 levels in nuclei isolated from day 32 control, C9orf72, and sALS iPSNs. Antibodies as indicated on right, genotype as indicated on bottom. Note: two independent CHMP7 antibodies were used. Lamin B1 was used as a loading control. n=8 control, 8 C9orf72, and 8 sALS iPSC lines. One-way ANOVA Tukey's multiple comparison test was used to calculate statistical significance. *** p<0.001, **** p<0.0001. Scale bar=5 μm.

FIG. 16 illustrates that nuclear localization of CHMP7 is increased in C9orf72 and sALS iPSNs and postmortem patient motor cortex (FIG. 16A). Immunostaining and confocal imaging for CHMP7 in iPSNs at day 25 of differentiation. Genotype as indicated on left, antibody as indicated on top (FIGS. 16B-16C). Quantification of nuclear/cytoplasmic ratio (FIG. 16B) and nuclear intensity (FIG. 16C) of CHMP7 immunostaining. n=7 control, 5 C9orf72, and 7 sALS iPSC lines, at least 50 Map2+ neurons per line. One-way ANOVA Tukey's multiple comparison test was used to calculate statistical significance. ** p<0.01, *** p<0.001 (FIG. 16D). Immunostaining for CHMP7 in paraffin embedded postmortem motor cortex. Genotype as indicated on left, antibody as indicated on top. Arrows indicate cytoplasmic CHMP7 immunostaining. Asterisks indicate nuclear CHMP7 immunostaining (FIGS. 16E-16F). Quantification of nuclear/cytoplasmic ratio (FIG. 16E) and nuclear intensity (FIG. 16F) of CHMP7 immunostaining. n=13 control, 17 C9orf72, and 30 sALS cases, at least 100 Map2+ cells per case. One-way ANOVA Tukey's multiple comparison test was used to calculate statistical significance. * p<0.05, *** p<0.001, **** p<0.0001. Scale bars=10 μm.

FIG. 17 illustrates that the inhibition of CHMP7 nuclear export in wildtype iPSNs recapitulates disease associated Nup alterations (FIG. 17A). Maximum intensity projections from SIM imaging of nuclei isolated from control iPSNs on day 25 of differentiation following 1 week of GFP tagged CHMP7 variant overexpression. Overexpression as indicated on left, antibodies as indicated on top (FIGS. 17B-17F). Quantification of CHMP7 and Nup spots and volume. n=4 control iPSC lines, 50 GFP+ nuclei per line/overexpression. One-way ANOVA with Tukey's multiple comparison test was used to calculate statistical significance. **** p<0.0001. Scale bar=5 μm.

FIG. 18 illustrates that the inhibition of CHMP7 nuclear export impacts TDP-43 localization and function (FIG. 18A). Immunostaining and confocal imaging for TDP-43 in control iPSNs on day 32 of differentiation following 2 weeks of GFP tagged CHMP7 variant overexpression. Overexpression as indicated on left, antibodies as indicated on top (FIG. 18B-FIG. 18C). Quantification of nuclear/cytoplasmic ratio (FIG. 18B) and nuclear intensity (FIG. 18C) of TDP-43 immunostaining. n=3 control iPSC lines, at least 50 Map2+ and GFP+ neurons per line/overexpression. One-way ANOVA with Tukey's multiple comparison test was used to calculate statistical significance. * p<0.05, **** p<0.0001 (FIGS. 18D-18E). qRT-PCR for full length (FIG. 18D) and truncated (FIG. 18E) stathmin-2 mRNA in control iPSNs on day 32 of differentiation following 2 weeks of GFP tagged CHMP7 variant overexpression. GAPDH was used for normalization. One-way ANOVA with Tukey's multiple comparison test was used to calculate statistical significance. * p<0.05, *** p<0.001. Scale bar=10 μm.

FIG. 19 illustrates that CHMP7 and TDP-43 co-pathology is present in a subset of neurons in postmortem patient motor cortex (FIG. 19A). Immunostaining for TDP-43 and CHMP7 in paraffin embedded postmortem motor cortex. Genotype as indicated on left, antibody as indicated on top. Arrows indicate cytoplasmic CHMP7 immunostaining. Asterisks indicate nuclear CHMP7 immunostaining (FIGS. 19B-19D). Quantification of nuclear/cytoplasmic ratio of TDP-43 vs cytoplasmic/nuclear ratio of CHMP7 in control (FIG. 19B), C9orf72 (FIG. 19C), and sALS (FIG. 19D) motor cortex. Individual data points for each Map2+ neuron analyzed are shown. n=10 control, 10 C9orf72, and 20 sALS cases. Note: red indicates high nuclear CHMP7/low nuclear TDP-43, yellow indicates high nuclear CHMP7/high nuclear TDP-43, blue indicates low nuclear CHMP7/low nuclear TDP-43, green indicates low nuclear CHMP7/high nuclear TDP-43. Scale bar=10 μm.

FIG. 20 illustrates that ASO mediated knockdown of CHMP7 restores the nuclear expression of specific Nups and mitigates TDP-43 mediated splicing defects and glutamate induced excitotoxicity in C9orf72 and sALS iPSNs. (FIG. 20A) Maximum intensity projections from SIM imaging of nuclei isolated from control, C9orf72, and sALS iPSNs on day 40 of differentiation following 2-week exposure to 51.1M scrambled control ASO or CHMP7 ASO 2. Treatment as indicated on left, genotype and antibodies as indicated on top (FIGS. 20B-20F). Quantification of spots and volume. n=6 control, 4 C9orf72, and 8 sALS iPSC lines, 50 NeuN+ nuclei per line/treatment. Two-way ANOVA with Tukey's multiple comparison test was used to calculate statistical significance. **** p<0.0001 (FIGS. 20G-qRT-PCR for full length (FIG. 20G) and truncated (FIG. 20H) stathmin-2 mRNA in control, C9orf72, and sALS iPSNs on day 46 of differentiation following 3-week exposure to 51.1M scrambled control ASO or CHMP7 ASO 2. GAPDH was used for normalization. n=8 control, 6 C9orf72, and 6 sALS iPSC lines. One-way ANOVA with Tukey's multiple comparison test was used to calculate statistical significance. *** p<0.001, **** p<0.0001 (FIG. 20I). Quantification of percent cell death as measured by propidium iodide (PI) incorporation following exposure to glutamate in control and C9orf72 iPSNs following 2-week exposure to 5 μM scrambled control ASO or CHMP7 ASO 2. n=7 control, 5 C9orf72, and 6 sALS iPSC lines, 5 frames per well. Two-way ANOVA with Tukey's multiple comparison test was used to calculate statistical significance. **** p<0.0001. Scale bar=5 μm.

FIG. 21 illustrates that CHMP7 is increased within the nucleus of C9orf72 and sALS iPSNs (FIGS. 21A-21B). Maximum intensity projections from SIM imaging of CHMP7 in nuclei isolated from control and sALS iPSNs at day 32 of differentiation. Genotype as indicated on left, antibody as indicated on top. Note: two independent CHMP7 antibodies were used. Single Z and inset images showing CHMP7 is primarily intra-nuclear but can associate in close-proximity with NPCs (as evaluated by Nup62 staining) in iPSNs. Scale bar=5 μm.

FIG. 22 illustrates that knockdown of POM121 does not result in increased nuclear CHMP7 (FIG. 22A). Maximum intensity projections from SIM imaging of CHMP7 and POM121 in nuclei isolated from control iPSNs following 48 hours of Trim21 GFP mediated POM121 knockdown. Antibody for knockdown as indicated on left, antibody for immunostaining as indicated on top (FIG. 22B). Quantification of POM121 and CHMP7 spots. n=3 control iPSC lines, 50 GFP+ nuclei per line. Student's t-test was used to calculate statistical significance. **** p<0.0001.

FIG. 23 illustrates that knockdown and overexpression of LEMD2 does not impact nuclear expression of CHMP7 or POM121 in iPSNs (FIGS. 23A-23B). Western blot (FIG. 23A) and quantification (FIG. 23B) of Trim21 GFP mediated reduction in LEMD2 in control iPSNs. n=3 control iPSC lines. Student's t-test was used to calculate statistical significance. * p<0.05 (FIG. 23C). Maximum intensity projections from SIM imaging of nuclei isolated from control iPSNs following Trim21 GFP mediated knockdown of LEMD2. Antibody used for knockdown as indicated on left, antibodies for immunostaining as indicated on top (FIGS. 23D-23G). Quantification of LEMD2, CHMP7, POM121, and 414 spots. n=3 control iPSC lines, 50 GFP+ nuclei per line/knockdown. Student's t-test was used to calculate statistical significance. **** p<0.0001 (FIG. 23F). Maximum intensity projections from SIM imaging of nuclei isolated from control iPSNs following 2 weeks of GFP tagged LEMD2 overexpression. Overexpression as indicated on left, antibodies for immunostaining as indicated on top (FIG. 231-FIG. 23L). Quantification of LEMD2, CHMP7, POM121, and 414 spots. n=3 control iPSC lines, 50 GFP+ nuclei per line/overexpression. Student's t-test was used to calculate statistical significance. * p<0.05, **** p<0.0001. Scale bars=5 μm.

FIG. 24 illustrates that CHMP7 ASOs reduce CHMP7 protein in iPSNs in a dose dependent manner (FIGS. 24A-24D). Western blot (FIG. 24A) and quantification (FIGS. 24B-24D) for CHMP7 protein in control (FIGS. 24A-24B), C9orf72 (FIG. 24A, FIG. 24C), and sALS (a,d) iPSNs following 2 week exposure to scrambled control or CHMP7 targeting ASOs. Genotype as indicated on top, concentration and ASO as indicated on bottom. n=6 control, 4 C9orf72, and 6 sALS iPSC lines. Two-way ANOVA with Tukey's multiple comparison test was used to calculate statistical significance. * p<0.05, *** p<0.001, **** p<0.0001.

FIG. 25 illustrates that ASO mediated knockdown of CHMP7 restores the localization of Ran GTPase to the nucleus in C9orf72 and sALS iPSNs (FIG. 25A). Immunostaining and confocal imaging for Ran GTPase in control, C9orf72, and sALS iPSNs on day 46 of differentiation following 3 weeks exposure to 5 μM scrambled control or CHMP7 targeting ASO. Treatment as indicated on left, genotype and antibodies as indicated on top (FIG. 25B). Quantification of nuclear/cytoplasmic ratio of Ran GTPase immunostaining. n=4 control, 4 C9orf72, and 6 sALS iPSC lines, at least 50 Map2+ neurons per line/treatment. Two-way ANOVA with Tukey's multiple comparison test was used to calculate statistical significance. **** p<0.0001. Scale bar=10 μm.

FIG. 26 illustrates that ASO mediated knockdown of CHMP7 restores the localization of TDP-43 to the nucleus in sALS iPSNs (FIG. 26A). Immunostaining and confocal imaging for TDP-43 in control, C9orf72, and sALS iPSNs on day 46 of differentiation following 3 weeks exposure to 5 μM scrambled control or CHMP7 targeting ASO. Treatment as indicated on left, genotype and antibodies as indicated on top. (FIG. 26B) Quantification of nuclear/cytoplasmic ratio of TDP-43 immunostaining. n=4 control, 4 C9orf72, and 6 sALS iPSC lines, at least 50 Map2+ neurons per line/treatment. Two-way ANOVA with Tukey's multiple comparison test was used to calculate statistical significance. **** p<0.0001. Scale bar=10 μm.

FIG. 27 shows that VPS4, but not CHMP4B nor CHMP2B, is increased in C9orf72 and sALS iPSN nuclei. FIGS. 27A-27B show the maximum intensity projections from SIM imaging of CHMP4B, CHMP2B, and VPS4 in nuclei isolated from control, C9orf72, and sALS iPSNs at day 18 (FIG. 27A) and 32 (FIG. 27B) of differentiation. Genotype is as indicated on left, time point and antibody as indicated on top. FIGS. 27C-27E shows the quantification of CHMP4B (FIG. 27C), CHMP2B (FIG. 27D), and VPS4 (FIG. 27E) spots. n=10 control, 10 C9orf72, and 10 sALS iPSC lines, 50 NeuN+nuclei per line/time point. One-way ANOVA with Tukey's multiple comparison test was used to calculate statistical significance. **p<0.01, ****p<0.0001. Scale bar=5 μm.

FIG. 28 shows that VPS4, but not CHMP4B nor CHMP2B, is increased in C9orf72 and sALS postmortem motor cortex neuronal nuclei. FIGS. 28A-28B show the maximum intensity projections from SIM imaging of CHMP4B, CHMP2B, and VPS4 in nuclei isolated from postmortem control, C9orf72, and sALS motor (FIG. 28A) and occipital (FIG. 28B) cortex tissue. Genotype as indicated on left, brain region and antibody as indicated on top. FIGS. 28C-FIG. 28E show quantification of CHMP4B (FIG. 28C), CHMP2B (FIG. 28D), and VPS4 (FIG. 28E) spots. n=6 control, 6 C9orf72, and 9 sALS cases, 50 NeuN+nuclei per line/brain region. One-way ANOVA with Tukey's multiple comparison test was used to calculate statistical significance. ****p<0.0001. Scale bar=5 μm.

FIG. 29 shows that the nuclear expression of VPS4 is dependent upon CHMP7 in C9orf72 and sALS iPSNs. FIGS. 29A, 29C and 29E show the maximum intensity projections from SIM imaging of CHMP4B (FIG. 29A), CHMP2B (FIG. 29C), and VPS4 (FIG. 29E) in nuclei isolated from control, C9orf72, and sALS iPSNs following 2-week exposure to 5 μM scrambled control or CHMP7 ASO. Treatment as indicated on left, genotype and antibody as indicated on top. FIGS. 29B, FIG. 29E and FIG. 29F show quantification of CHMP4B (FIG. 29B), CHMP2B (FIG. 29D), and VPS4 (FIG. 29F) spots. n=5 control, 5 C9orf72, and 5 sALS iPSC lines, 50 NeuN+nuclei per line/treatment. Two-way ANOVA with Tukey's multiple comparison test was used to calculate statistical significance. **p<0.01, ****p<0.0001. Scale bar=5 μm.

FIG. 30 shows the overexpression of a dominant negative VPS4 increases nuclear POM121 spots but does not restore their distribution in C9orf72 and sALS iPSNs. FIG. 30A and FIG. 30C show the maximum intensity projections from SIM imaging of VPS4 (FIG. and POM121 (FIG. 30C) in nuclei isolated from control, C9orf72, and sALS iPSNs overexpressing GFP or GFP tagged VPS4 variants. Overexpression as indicated on left, genotype and antibody as indicated on top. Arrows (FIG. 30C) indicate uneven distribution of POM121 observed following overexpression of dominant negative VPS4 (VPS4^{E228Q}) in ALS nuclei. FIG. 30B and FIG. 30D show the quantification of VPS4 (FIG. 30B) and POM121 (FIG. 30D) spots. n=4 control, 4 C9orf72, and 4 sALS iPSC lines, 50 GFP+nuclei per line/overexpression. Two-way ANOVA with Tukey's multiple comparison test was used to calculate statistical significance. *p<0.05, **p<0.01, ****p<0.0001. Scale bar=5 μm.

FIG. 31 shows the expression of VPS4 is increased in C9orf72 and sALS iPSN nuclei but not whole iPSN lysates (FIGS. 31A-31D). Western blot (FIG. 30A) and quantification (FIGS. 31B-31D) for CHMP4B (FIG. 31A-31B), CHMP2B (FIGS. 31A, 31C), and VPS4 (FIGS. 31A, 31D) expression in control, C9orf72, and sALS iPSN lysates. Antibodies as indicated on right, genotype as indicated on bottom. GAPDH was used as a loading control. n=4 control, 4 C9orf72, and 4 sALS iPSC lines. One-way ANOVA with Tukey's multiple comparison test was used to calculate statistical significance (FIGS. 31E-31H). Western blot (FIG. 31E) and quantification (FIG. 31F-31H) for CHMP4B (FIGS. 3L-31F), CHMP2B (FIGS. 31E, 31G), and VPS4 (FIGS. 31E, 31H) expression in nuclei isolated from control, C9orf72, and sALS iPSNs. Antibodies as indicated on right, genotype as indicated on bottom. Lamin B1 was used as a loading control. n=4 control, 4 C9orf72, and 4 sALS iPSC lines. One-way ANOVA with Tukey's multiple comparison test was used to calculate statistical significance. ** p<0.01.

### DETAILED DESCRIPTION

Section headings as used in this section and the entire disclosure herein are merely for organizational purposes and are not intended to be limiting.

### 1. Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially o” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “administering” as used herein means providing a pharmaceutical agent or composition, such as a CHMP7 inhibiting agent (e.g., such as a CHMP7 antisense oligonucleotide), to a subject, and includes, but is not limited to, administering by a medical professional, self-administering, or a combination thereof. As used herein, the term “treating” a disease in a subject or “treating” a subject having a disease refers to subjecting the subject to a pharmaceutical treatment, e.g., the administration of a drug, such as a CHMP7 inhibiting agent (e.g., such as a CHMP7 antisense oligonucleotide or a pharmaceutically acceptable salt of an antisense oligonucleotide), or siRNA, such that at least one symptom of the disease (e.g., such as a neurodegenerative disease) is decreased or prevented from worsening.

The term “antibody” as used herein, refers to a molecule characterized by reacting specifically with an antigen in some way, where the antibody and the antigen are each defined in terms of the other. Antibody may refer to a complete antibody molecule or any fragment or region thereof, such as the heavy chain, the light chain, Fab region, and Fc region.

The term antigen-binding fragment” as used herein refers to antibody fragments that retain the ability to bind specifically to the antigen bound by the full-length antibody, e.g. fragments that retain one or more CDR regions. Examples of antigen-binding fragments include, but are not limited to, Fab, Fab′, F(ab′)₂, and Fv fragments; diabodies; single-chain antibody molecules, e.g., sc-Fv; nanobodies and multispecific antibodies formed from antibody fragments.

The term “CHMP7” or “charged multivesicular body protein 7” as used interchangeably herein, refers to a human ESCRT-III-related protein 453 amino acids in length (see, GENBANK Accession No. Q8WUX9). CHMP7 contains an SNF7 domain and a distantly SNF7-related domain in its C-terminal half and N-terminal half. The term “CHMP7” as used herein refers not only to the 453 amino acid protein, but also to fragments of this protein (e.g., such fragments can have a length of 5 amino acids to 400 amino acids, 10 amino acids to 350 amino acids, 10 amino acids to 300 amino acids, 10 amino acids to 250 amino acids, 10 amino acids to 200 amino acids, 10 amino acids to 150 amino acids, 10 amino acids to 100 amino acids, 10 amino acids to 75 amino acids, 10 amino acids to 50 amino acids, 10 amino acids to 40 amino acids, 10 amino acids to 30 amino acids, or 10 amino acids to 20 amino acids) as well as CHMP7 DNA, CHMP7 cDNA, CHMP7 RNA, etc.

The term “CHMP7 inhibiting agent” as used herein refers to any compound or molecule that is capable of inhibiting or reducing CHMP7 (such as a CHMP7 protein or fragment thereof (e.g, such fragments can have a length of 5 amino acids to 400 amino acids, 10 amino acids to 350 amino acids, 10 amino acids to 300 amino acids, 10 amino acids to 250 amino acids, 10 amino acids to 200 amino acids, 10 amino acids to 150 amino acids, 10 amino acids to 100 amino acids, 10 amino acids to 75 amino acids, 10 amino acids to 50 amino acids, 10 amino acids to 40 amino acids, 10 amino acids to 30 amino acids, or 10 amino acids to 20 amino acids), CHMP7 DNA, CHMP7 cDNA, CHMP7 siRNA, CHMP7 miRNA, etc.) in a cell or population of cells, such as a neuronal cell or population of neuronal cells. Examples of CHMP7 inhibiting agents include, but are not limited to, one or more of a small molecule (including pharmaceutically acceptable salts thereof), an antibody (such as a monoclonal antibody, a chimeric antibody, a humanized antibody, a fully human antibody, etc.) or antigen binding fragments thereof, an oligonucleotide (e.g., antisense oligonucleotide or a pharmaceutically acceptable salt of an antisense oligonucleotide), a microRNA (miRNA), small-interfering RNA (siRNA), a peptide, a peptidomimetic, or any combinations thereof.

The term “contiguous” as used herein means immediately adjacent to each other.

The term “immediately adjacent” as used herein means there are no intervening elements between the immediately adjacent elements.

The term “inhibit” or “inhibits” means to decrease, suppress, attenuate, diminish, arrest, or stabilize the development or progression of a disease, disorder, or condition, the activity of a biological pathway, or a biological activity, such as the growth of a solid malignancy, e.g., by at least 10%, 20%, 30%, 40%, 50%, 60%, 70%, 80%, 90%, 95%, 98%, 99%, or even 100% compared to an untreated control subject, cell, biological pathway, or biological activity or compared to the target, such as a growth of a solid malignancy, in a subject before the subject is treated. By the term “decrease” is meant to inhibit, suppress, attenuate, diminish, arrest, or stabilize a symptom of a cancer disease, disorder, or condition. It will be appreciated that, although not precluded, treating a disease, disorder or condition does not require that the disease, disorder, condition or symptoms associated therewith be completely eliminated.

The phrase, “neurodegenerative disease” as used herein, refers to a disorder (including a neuropathy) associated with degeneration or dysfunction of neurons or other neural cells such as retinal ganglion cells. A neurodegenerative disease or disorder can be any disease or condition in which decreased function or dysfunction of neurons, or loss or neurons or other neural cells, can occur. Such conditions include, without limitation, glaucoma, and neurodegenerative disorders such as or associated with alcoholism, Alexander's disease, Alper's disease, Alzheimer's disease, Amyotrophic Lateral Sclerosis (Lou Gehrig's Disease), ataxia telangiectasia, Batten disease (also known as Spielmeyer-Vogt-Sjogren-Batten disease), bovine spongiform encephalopathy (BSE), Canavan disease, Cockayne syndrome, corticobasal degeneration, Creutzfeldt-Jakob disease, diabetic neuropathy, frontotemporal degeneration (FTD), Huntington's disease, HIV-associated dementia, Kennedy's disease, Krabbe's disease, Lewy body dementia, neuroborreliosis, Machado-Joseph disease (Spinocerebellar ataxia type 3), wet or dry macular degeneration, Multiple System Atrophy, multiple sclerosis, Niemann Pick disease, Parkinson's disease, Pelizaeus-Merzbacher Disease, photoreceptor degenerative diseases such as retinitis pigmentosa and associated diseases, Pick's disease, primary lateral sclerosis, prion diseases, Progressive Supranuclear Palsy, Refsum's disease, Sandhoffs disease, Schilder's disease, subacute combined degeneration of spinal cord secondary to pernicious anemia, Spielmeyer-Vogt-Sjogren-Batten disease (also known as Batten disease), spinocerebellar ataxia (multiple types with varying characteristics), spinal muscular atrophy, Steele-Richardson-Olszewski disease, and tabes dorsalis. Traumatic injury or other damage to neuronal cells (e.g., trauma due to accident, blunt-force injury, gunshot injury, spinal cord injury, ischemic conditions of the nervous system such as stroke, cell damage due to aging or oxidative stress, and the like) is also intended to be included within the language “neurodegenerative disease or disorder”. In certain embodiments, the neurodegenerative disease or disorder is a disease or disorder that is not associated with excessive angiogenesis, for example, glaucoma that is not neovascular glaucoma.

In some aspects, the neurodegenerative disease is Amyotrophic Lateral Sclerosis (Lou Gehrig's Disease). In other aspects, the neurodegenerative disease is Huntington's disease. In another aspect, the neurodegenerative disease is Alzheimer's disease. In another aspect, the neurodegenerative disease is Parkinson's disease. In yet another aspect, the neurodegenerative disease is Lewy body dementia. In still yet another aspect, the neurodegenerative disease is multiple sclerosis. In still yet another aspect, the neurodegenerative disease is frontotemporal degeneration (FTD).

The term “nucleic acid” as used herein, refers to molecules composed of monomeric nucleotides. A nucleic acid includes, but is not limited to, ribonucleic acids (RNA), deoxyribonucleic acids (DNA), single-stranded nucleic acids, double-stranded nucleic acids, small-interfering ribonucleic acids (siRNA), and microRNAs (miRNA).

As used herein, the term “nucleobase” means a heterocyclic moiety capable of pairing with a base of another nucleic acid.

The term “oligonucleotide” as used herein, refers to an oligomer or polymer of ribonucleic acid or deoxyribonucleic acid. This term includes oligonucleotides composed of naturally-occurring nucleobases, sugars and covalent intersugar (backbone) linkages as well as oligonucleotides having non-naturally-occurring portions which function similarly. Such modified or substituted oligonucleotides are often preferred over native forms because of desirable properties such as, for example, enhanced cellular uptake, enhanced binding to target and increased stability in the presence of nucleases. The oligonucleotides of the present disclosure comprise from about 5 to about 50 contiguous nucleobases. In another aspect, the oligonucleotides comprise from about 8 to about 50 contiguous nucleobases. In yet another aspect, the oligonucleotides comprise from about 10 to about 50 contiguous nucleobases. In still yet another aspect, the oligonucleotides comprise from about 15 to about contiguous nucleobases. In still a further aspect, the oligonucleotides comprise from about 20 to about 50 contiguous nucleobases.

An example of an oligonucleotide is an antisense contiguous oligonucleotide. The antisense oligonucleotides of the present disclosure, including pharmaceutically acceptable salts thereof, comprise from about 5 to about 50 contiguous nucleobases. In another aspect, the antisense oligonucleotides comprise from about 8 to about 50 contiguous nucleobases. In yet another aspect, the antisense oligonucleotides comprise from about 10 to about 50 contiguous nucleobases. In still yet another aspect, the antisense oligonucleotides comprise from about 15 to about 50 contiguous nucleobases. In still a further aspect, the antisense oligonucleotides comprise from about 20 to about 50 contiguous nucleobases.

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2=, 3= or 5=hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure, however, open linear structures are generally preferred. Within the oligonucleotide structure, the phosphate groups are commonly referred to as forming the internucleoside backbone of the oligonucleotide. The normal linkage or backbone of RNA and DNA is a 3= to 5=phosphodiester linkage.

Additionally, the antisense oligonucleotides of the present disclosure or pharmaceutically acceptable salts thereof may be single stranded or double-stranded (such as dsRNA molecules).

The terms “parenteral administration” and “administered parenterally” as used herein mean modes of administration other than enteral and topical administration, usually by injection, and includes, without limitation, intravenous, intramuscular, intarterial, intrathecal, intracapsular, intraorbital, intraocular, intracardiac, intradermal, intraperitoneal, transtracheal, subcutaneous, subcuticular, intraarticular, subcapsular, subarachnoid, intraspinal and intrasternal injection and infusion.

The term “peptide” means a molecule formed by linking at least two amino acids by amide bonds. Without limitation, as used herein, peptide refers to polypeptides and proteins. Peptides for use in the present disclosure can have a length of 5 amino acids to 500 amino acids, 10 amino acids to 400 amino acids, 10 amino acids to 300 amino acids, 10 amino acids to 200 amino acids, 10 amino acids to 100 amino acids, 10 amino acids to 90 amino acids, 10 amino acids to 80 amino acids, 10 amino acids to 70 amino acids 10 amino acids to 60 amino acids, 10 amino acids to 50 amino acids or 10 amino acids to 40 amino acids. A “peptidomimetic” is a type of peptide that includes unnatural or synthetic amino acids, including D and L isomers and amino acid analogs linked by amide linkages or other bonds, e.g., ester, ether, etc. “Peptidomimetics” also include organic molecules not obviously analogous to peptides, including, for example, aptamers.

The term “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable carrier” as used herein means a pharmaceutically acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

The term “pharmaceutically acceptable salts” refers to the relatively non-toxic, inorganic and organic salts of compounds, including small molecules and oligonucleotides, such as antisense oligonucleotides

The terms “prevent,” “preventing,” “prevention,” “prophylactic treatment,” and the like refer to reducing the probability of developing a disease, disorder, or condition in a subject, who does not have, but is at risk of or susceptible to developing a disease, disorder, or condition

A “subject” can include a human subject for medical purposes, such as for the treatment of an existing disease, disorder, condition or the prophylactic treatment for preventing the onset of a disease, disorder, or condition or an animal subject for medical, veterinary purposes, or developmental purposes. Suitable animal subjects include mammals including, but not limited to, primates, e.g., humans, monkeys, apes, gibbons, chimpanzees, orangutans, macaques and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, guinea pigs, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a disease, disorder, or condition. Thus, the terms “subject” and “patient” are used interchangeably herein. Subjects also include animal disease models (e.g., rats or mice used in experiments, and the like).

The term “subject suspected of having” means a subject exhibiting one or more clinical indicators of a disease or condition. In certain embodiments, the disease or condition is a neurodegenerative disease.

The term “subject in need thereof” means a subject identified as in need of a therapy or treatment.

The terms “systemic administration,” “administered systemically,” “peripheral administration,” and “administered peripherally” mean the administration of a compound, drug or other material other than directly into the central nervous system, such that it enters the patient's system and, thus, is subject to metabolism and other like processes, for example, subcutaneous administration.

The term “therapeutically effective amount” or “effective amount” as used interchangeably herein refers to an amount sufficient to produce the desired effect, e.g., to reduce or ameliorate the severity, duration, progression, or onset of a disease, condition, or disorder (e.g., a disease, condition, or disorder related to loss of neuronal cells or cell function, such as for example, a neurodegenerative disease), or one or more symptoms thereof; prevent the advancement of a disease, condition, or disorder; cause the regression of a disease, condition, or disorder; prevent the recurrence, development, onset or progression of a symptom associated with a disease, condition, or disorder; or enhance or improve the prophylactic or therapeutic effect(s) of another therapy.

In some aspects, the effective amount or therapeutically effective amount of a compound (such as a CHMP7 inhibiting agent) will depend on its therapeutic index, solubility, and the like. For example, certain compounds discovered by the methods of the present disclosure may be administered in a sufficient amount to produce a reasonable benefit/risk ratio applicable to such treatment. By way of example, a CHMP7 inhibiting agent may be administered in a sufficient amount to produce a reasonable benefit/risk ratio applicable to such treatment.

In some other aspects, an effective amount or therapeutically effective amount of a CHMP7 inhibiting agent (e.g., such as a CHMP7 antisense oligonucleotide and/or siRNA) according to this disclosure can range from, e.g., about 0.001 mg/kg to about 1000 mg/kg, or in certain embodiments, about 0.01 mg/kg to about 100 mg/kg, or in certain embodiments, about 0.1 mg/kg to about 50 mg/kg. Effective doses will also vary, as recognized by those skilled in the art, depending on the disorder treated, route of administration, excipient usage, the age and sex of the subject, and the possibility of co-usage with other therapeutic treatments such as use of other agents.

The term “therapeutic effect” refers to a local or systemic effect in animals, particularly mammals, and more particularly humans, caused by a pharmacologically active substance, such as, for example a CHMP7 inhibiting agent. The term thus means any substance (e.g., such as a CHMP7 inhibiting agent) intended for use in the diagnosis, cure, mitigation, treatment or prevention of disease or in the enhancement of desirable physical or mental development and conditions in an animal, such as a human.

The term “treating” a disease in a subject or “treating” a subject having a disease refers to subjecting the subject to a pharmaceutical treatment, e.g., the administration of a drug, such that at least one symptom of the disease is decreased or prevented from worsening.

The following examples have been included to provide guidance to one of ordinary skill in the art for practicing representative embodiments of the presently disclosed subject matter. In light of the present disclosure and the general level of skill in the art, those of skill can appreciate that the following examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### 2. Screening Methods

In one embodiment, the present disclosure relates to methods for screening compounds which: (a) bind to CHMP7 (such as a CHMP7 protein, CHMP7 DNA, CHMP7 cDNA, CHMP7 RNA, etc.) with a certain binding affinity; (b) inhibit the polymerization of CHMP7, (c) inhibit the intranuclear accumulation of CHMP7 and/or (d) reduce or inhibit CHMP7 expression in at least one cell or a population of cells, such as, for example, at least one neuronal cell or a population of neuronal cells or a population of other nervous system cell such as astrocytes, oligodendroglia or microglia. Compounds that can be screened and selected pursuant to the methods described herein are at least one of a small molecule or a pharmaceutically acceptable salt thereof, an antibody (such as a monoclonal antibody, a chimeric antibody, a humanized antibody, a fully human antibody, etc.) or antigen-binding fragments thereof, an oligonucleotide (such as an antisense oligonucleotide or a pharmaceutically acceptable salt thereof), a small-interfering RNA (siRNA), a microRNA (miRNA), a peptide, a peptidomimetic, or any combinations thereof.

In one aspect, the screening method involves measuring the binding of a compound to CHMP7. In this method, the compound to be tested is contacted with CHMP7 (such as a CHMP7 protein or fragment thereof, CHMP7 DNA, CHMP7 cDNA, CHMP7 RNA, etc.) and the binding (or lack thereof) measured using routine techniques known in the art. A compound may be identified (and selected) as a compound that binds to CHMP7 (such as a CHMP7 protein or fragment thereof, CHMP7 DNA, CHMP7 cDNA, CHMP7 RNA, etc.) if it has a particular binding affinity to CHMP7. In some aspects, the compound may be identified as a compound that specifically binds to CHMP7 (such as a CHMP7 protein, CHMP7 DNA, CHMP7 cDNA, CHMP7 RNA, etc.). For example, a compound that binds to CHMP7 may have a dissociation constant (Kd) in the micromolar range; or, preferably, in the range of 100 nM to 1 pM. As mentioned, the binding of a compound to CHMP7 can be measured by using routine techniques in the art, such as, for example, Biacore, ChemoProteomics, Microscale Thermophoresis or any other techniques or combination of techniques known in the art.

In another aspect, the screening method involves contacting a compound with at least one cell or population of cells (e.g., a neuronal cell or population of neuronal cells) that expresses CHMP7 and determining whether the compound reduces or inhibits CHMP7 expression in the cell or population of cells. The expression of CHMP7 in the presence and absence of the test compound may be measured using routine techniques in the art, such as, for example, (q)PCR, Western Blot, Mass spectroscopy or any other techniques or combination of techniques known in the art. Compounds that reduce or inhibit CHMP7 can be selected for further testing and use in other methods described herein.

The screening methods described herein may additionally comprise the step of comparing the compound being tested to a control. Said control may be an inactive test compound, wherein said inactive test compound is a compound that (a) does not reduce the expression and/or activity of CHMP7; and/or (b) does not bind to CHMP7.

In another aspect, a compound that reduces the expression of CHMP7 in a cell or population of cells (e.g., a neuronal cell or population of neuronal cells) is identified (and can be selected) as a compound that prevents, ameliorates and/or inhibits (i.e., treats) a neurodegenerative disease. In another aspect, a compound that reduces the activity of CHMP7 is identified (and can be selected) as a compound that prevents, ameliorates and/or inhibits (i.e., treats) a neurodegenerative disease.

The above screening methods can lead to the identification of a compound that prevents, ameliorates and/or inhibits a neurodegenerative disease. Such compounds can be selected and used to ameliorate and/or inhibit (namely, treats) a neurodegenerative disease. Thus, the herein provided screening methods are useful in the identification and selection of compounds for treating a neurodegenerative disease.

### 3. CHMP7 Inhibiting Agent and Pharmaceutical Compositions Containing CHMP7 Inhibiting Agents

In another embodiment, the present disclosure relates to at least one CHMP7 inhibiting agent. A CHMP7 inhibiting agent can be a compound which binds to CHMP7 (such as a CHMP7 protein, CHMP7 DNA, CHMP7 cDNA, CHMP7 RNA, etc.) with a certain binding affinity; and/or (b) reduces or inhibits CHMP7 (such as a CHMP7 protein, CHMP7 DNA, CHMP7 cDNA, CHMP7 RNA, etc.) expression in at least one cell or a population of cells. Such compounds can include the compounds identified and selected in Section 2, herein. In another aspect, the CHMP7 inhibiting agent is a CHMP7 inhibiting agent known in the art.

The CHMP7 inhibiting agent can be least one of a small molecule or a pharmaceutically acceptable salt thereof, an antibody (such as a monoclonal antibody, a chimeric antibody, a humanized antibody, a fully human antibody, etc.) or antigen-binding fragment thereof, an oligonucleotide (such as an antisense oligonucleotide or a pharmaceutically acceptable salt thereof), a small interfering RNA (siRNA), a microRNA (miRNA), a peptide, a peptidomimetic, or any combinations thereof. In one aspect, the CHMP7 inhibiting agent is a small molecule or a pharmaceutically acceptable salt thereof. In another aspect, the CHMP7 inhibiting agent is an antibody or an antigen-binding fragment thereof. In yet another aspect, the CHMP7 inhibiting agent is an oligonucleotide. In yet another aspect, the CHMP7 inhibiting agent is an antisense oligonucleotide or a pharmaceutically acceptable salt thereof. In still another aspect, the CHMP7 inhibiting agent is a siRNA. In still another aspect, the CHMP7 inhibiting agent is a small interfering RNA. In still another aspect, the CHMP7 inhibiting agent is a microRNA. In yet a further aspect, the CHMP7 inhibiting agent is a peptide. In yet still a further aspect, the CHMP7 inhibiting agent is a peptidometric. In some aspects, the CHMP7 inhibiting agent is an oligonucleotide. In another aspect, the CHMP7 inhibiting agent is an antisense oligonucleotide or a pharmaceutically acceptable salt thereof. Examples of antisense oligonucleotides which are CHMP7 inhibiting agents and can be used in the methods described herein include those described in Coyne et al., “Nuclear accumulation of CHMP7 initiates nuclear pore complex injury and subsequent TDP-43 dysfunction in sporadic and familial AILS” Science Translational Medicine, Vol. 13, No. 604 (2021) and include one or more of the following: CCTATAGGACTATCCAGGAA (SEQ ID NO:2), GAAAACGGTTTCCACTGTAT (SEQ ID NO:3), TGTTACCCTCAGATACCGCC (SEQ ID NO:4), ATGTGATGCTATTAATAGGA (SEQ ID NO:14) and any combinations thereof.

In some aspects, the CHMP7 inhibiting agent is a siRNA. Examples of siRNA sequences which are CHMP7 inhibiting agents include one or more of the following:
siRNA: CGACCUUGGUAAACGGAAA (SEQ ID NO:22)
siRNA: GGGUUUAUCCUGUCGCUAA (SEQ ID NO:23)
siRNA: GGAGGUGUAUCGUCUGUAU (SEQ ID NO:24)
siRNA: GUAACAAAUGGCUUAGAUU (SEQ ID NO:25)

In another aspect, the present disclosure provides pharmaceutically acceptable compositions which comprise a therapeutically effective amount of one or more such CHMP7 inhibiting agents (e.g., an active agent or first active agent) described above, formulated together with one or more pharmaceutically acceptable carriers (additives) and/or diluents.

As described in detail below, the pharmaceutical compositions of the present disclosure may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous, intrathecal, or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

CHMP7 inhibiting agent compositions include those suitable for oral, nasal, topical (including buccal and sublingual), rectal, vaginal and/or parenteral administration. The formulations may conveniently be presented in unit dosage form and may be prepared by any methods well known in the art of pharmacy. The amount of CHMP7 inhibiting agent which can be combined with a carrier material to produce a single dosage form will vary depending upon the host being treated and the particular mode of administration. The amount of CHMP7 inhibiting agent which can be combined with a carrier material to produce a single dosage form will generally be that amount of the compound which produces a therapeutic effect.

In certain embodiments, a formulation of CHMP7 inhibiting agent compositions can comprise other carriers to allow more stability, to allow more stability, different releasing properties in vivo, targeting to a specific site, or any other desired characteristic that will allow more effective delivery of the complex to a subject or a target in a subject, such as, without limitation, liposomes, microspheres, nanospheres, nanoparticles, bubbles, micelle forming agents, e.g., bile acids, and polymeric carriers, e.g., polyesters and polyanhydrides. In certain embodiments, an aforementioned composition renders orally bioavailable the CHMP7 inhibiting agent of the present disclosure.

Liquid dosage formulations of CHMP7 inhibiting agent compositions include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of an active ingredient. A CHMP7 inhibiting agent composition of the present disclosure may also be administered as a bolus, electuary or paste.

In solid dosage forms (e.g., capsules, tablets, pills, dragees, powders, granules and the like), the is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol, glycerol monostearate, and non-ionic surfactants; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. Compositions may also be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Formulations for rectal or vaginal administration may be presented as a suppository, which may be prepared by mixing one or more compounds of the disclosure with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations which are suitable for vaginal administration also include pessaries, tampons, creams, gels, pastes, foams or spray formulations containing such carriers as are known in the art to be appropriate.

Dosage forms for the topical or transdermal administration of a CHMP7 inhibiting agent composition of the present disclosure include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The ointments, pastes, creams and gels may contain, in addition to an active compound of this disclosure, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this disclosure.

Pharmaceutical compositions suitable for parenteral administration can comprise sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

In certain embodiments, the above-described pharmaceutical compositions can be combined with other pharmacologically active compounds (“second active agents”) known in the art according to the methods and compositions provided herein. Second active agents can be large molecules (e.g., proteins) or small molecules (e.g., synthetic inorganic, organometallic, or organic molecules). In one embodiment, second active agents independently or synergistically help to treat cancer. Examples of second active agents with mechanism independent of CHMP7 inhibition include one or more of immunotherapy, hormonal therapy, radiation therapy, HER2/neu receptor antagonists, vincristine, cyclophosphamide, cytarabine, etoposide, and/or doxarubicin.

### 4. Methods of Inhibiting CHMP7 Expression

In another embodiment, the present disclosure relates to methods of inhibiting CHMP7 expression in a cell or population of cells, such as a neuronal cell or population of neuronal cells, in a subject in need of treatment thereof. In one aspect, the method involves administering to a cell or population of cells an effective amount or therapeutically effective amount of at least one CHMP7 inhibiting agent or a composition comprising an effective amount or therapeutically effective amount of at least one CHMP7 inhibiting agent to a subject in need of treatment thereof and then determining, detecting and/or measuring the inhibition of CHMP7 expression using routine techniques known in the art, such as, for example, (q)PCR, Western Blot, Mass spectroscopy or any other techniques or combination of techniques known in the art. In some aspects, the subject is suffering from at least one neurodegenerative disease.

An effective amount or therapeutically effective amount of a CHMP7 inhibiting agent (e.g., such as a CHMP7 antisense oligonucleotide and/or siRNA) or a composition containing an effective amount or a therapeutically effective amount of a CHMP7 inhibiting agent used in said methods can range from about 0.001 mg/kg to about 1000 mg/kg, or in certain embodiments, about 0.01 mg/kg to about 100 mg/kg, or in certain embodiments, about 0.1 mg/kg to about 50 mg/kg.

The effective amount or therapeutically effective amount of a CHMP7 inhibiting agent (e.g., such as a CHMP7 antisense oligonucleotide and/or siRNA) or a composition containing an effective amount or a therapeutically effective amount of a CHMP7 inhibiting agent used in the methods herein can be administered orally, parenterally, topically, intravaginally or intrarectally, sublingually, ocularly, transdermally or nasally, using routine techniques known in the art.

### 4. Methods of Treating Neurodegenerative Diseases

In another embodiment, the present disclosure relates to methods of treating at least one neurodegenerative disease in a subject in need of treatment thereof. In one aspect, the method involves administering to a subject suffering or believed to be suffering from at least one neurodegenerative disease (and in need of treatment thereof), an effective amount or therapeutically effective amount of at least one CHMP7 inhibiting agent or a composition comprising an effective or therapeutically effective amount of at least one CHMP7 inhibiting agent to treat the neurodegenerative disease. Any neurodegenerative disease can be treated pursuant to the methods described herein. In some aspects, the neurodegenerative disease is Amyotrophic Lateral Sclerosis (Lou Gehrig's Disease). In other aspects, the neurodegenerative disease is Huntington's disease. In another aspect, the neurodegenerative disease is Alzheimer's disease. In another aspect, the neurodegenerative disease is Parkinson's disease. In yet another aspect, the neurodegenerative disease is Lewy body dementia. In still yet another aspect, the neurodegenerative disease is multiple sclerosis. In still yet another aspect, the neurodegenerative disease is frontotemporal degeneration (FTD).

An effective amount or therapeutically effective amount of a CHMP7 inhibiting agent (e.g., such as a CHMP7 antisense oligonucleotide and/or siRNA) or a composition containing an effective amount or a therapeutically effective amount of a CHMP7 inhibiting agent used in said methods can range from about 0.001 mg/kg to about 1000 mg/kg, or in certain embodiments, about 0.01 mg/kg to about 100 mg/kg, or in certain embodiments, about 0.1 mg/kg to about 50 mg/kg.

The effective amount or therapeutically effective amount of a CHMP7 inhibiting agent (e.g., such as a CHMP7 antisense oligonucleotide and/or siRNA) or a composition containing an effective amount or a therapeutically effective amount of a CHMP7 inhibiting agent used in the methods herein can be administered orally, parenterally, topically, intravaginally or intrarectally, sublingually, ocularly, transdermally or nasally, using routine techniques known in the art.

### EXAMPLES

### Example 1

Materials and Methods

The below materials and methods were used for Examples 2-7

iPSC Derived Neuronal Differentiation

C9orf72 and non-neurological control iPSC lines were obtained from the Answer ALS repository at Cedars-Sinai (See Table A for demographics). Feeder-free iPSCs were maintained on Matrigel with MTeSR and maintained according to Cedars Sinai SOP. iPSCs were differentiated into spinal neurons using the previously described direct induced motor neuron (diMNs) protocol (6). All cells were maintained at 37° C. with 5% CO2. iPSCs and iPSNs routinely tested negative for mycoplasma.

TABLE A
Demographic information for iPSC lines

| iPSC | | | Age at | | |
|---|---|---|---|---|---|
| Line | | | Time of | | |
| Name | Source | Clinical Diagnosis | Collection | Sex | Origin |
| EDi036-A | Cedars- | Non-neurologic control | 79 | Female | PBMC |
| | Sinai | | | | |
| EDi037-A | Cedars- | Non-neurologic control | 79 | Male | PBMC |
| | Sinai | | | | |
| EDi029-A | Cedars- | Non-neurologic control | 80 | Male | PBMC |
| | Sinai | | | | |
| EDi034-A | Cedars- | Non-neurologic control | 79 | Female | PBMC |
| | Sinai | | | | |
| EDi022-A | Cedars- | Non-neurologic control | 79 | Male | PBMC |
| | Sinai | | | | |
| EDi044-A | Cedars- | Non-neurologic control | 80 | Female | PBMC |
| | Sinai | | | | |
| CS1ATZ | Cedars- | Non-neurologic control | 60 | Male | PBMC |
| | Sinai | | | | |
| CS8PAA | Cedars- | Non-neurologic control | 58 | Female | PBMC |
| | Sinai | | | | |
| EDi043-A | Cedars- | Non-neurologic control | 80 | Male | PBMC |
| | Sinai | | | | |
| CS0201 | Cedars- | Non-neurologic control | 56 | Female | PBMC |
| | Sinai | | | | |
| CS0002 | Cedars- | Non-neurologic control | 51 | Male | PBMC |
| | Sinai | | | | |
| CS9XH7 | Cedars- | Non-neurologic control | 53 | Male | PBMC |
| | Sinai | | | | |
| CS0BUU | Cedars- | C9orf72 | 63 | Female | PBMC |
| | Sinai | | | | |
| CS7VCZ | Cedars- | C9orf72 | 64 | Male | PBMC |
| | Sinai | | | | |
| CS0LPK | Cedars- | C9orf72 | 67 | Male | PBMC |
| | Sinai | | | | |
| CS6ZLD | Cedars- | C9or172 | | Female | PBMC |
| | Sinai | | | | |
| CS8KT3 | Cedars- | C9orf72 | 60 | Male | PBMC |
| | Sinai | | | | |
| CS2YNL | Cedars- | C9orf72 | 60 | Male | PBMC |
| | Sinai | | | | |
| CS0NKC | Cedars- | C9orf72 | 52 | Female | PBMC |
| | Sinai | | | | |
| CS6CLW | Cedars- | C9orf72 | | Male | PBMC |
| | Sinai | | | | |

Leptomycin B and ASO Treatment of iPSC Derived Neurons Leptomycin B: On day 18 of differentiation, 10 nM Leptomycin B (LMB, Cayman Chemicals) was added to the culture media and iPSNs were returned to the incubator for ˜18 hours before downstream analyses were conducted. Ethanol was used as vehicle control.

C9orf72 repeat ASO: Sense strand targeting G4C2 ASO (619251) (13) was generously provided by Ionis Pharmaceuticals. On day 20 of differentiation, 5 μM sense targeting ASO was added to the culture media. Media was exchanged and ASO replaced on day 22 of differentiation and downstream analyses were performed on day 25 of differentiation. CHMP7 ASO: Non-targeting scrambled control (676630): CCTATAGGACTATCCAGGAA (SEQ ID NO: 2), CHMP7 ASO 1 (1508916): GAAAACGGTTTCCACTGTAT (SEQ ID NO: 3), CHMP7 ASO 2 (1508917): TGTTACCCTCAGATACCGCC (SEQ ID NO: 4), and CHMP7 ASO 3 (1508918): ATGTGATGCTATTAATAGGA (SEQ ID NO:14) were generously provided by Ionis Pharmaceuticals. On day 25 of differentiation, ASOs were added to the culture media at concentrations of 1, 5, or 10 μM as indicated in Description of the Figures. Media was exchanged and ASO replaced every 3 days until day 40 of differentiation.

Nuclei Isolation

Nuclei were isolated from iPSNs and postmortem human brain and spinal cord tissue using the Nuclei Pure Prep Nuclei Isolation Kit (Sigma Aldrich) following manufacturer protocol as previously described (13). Briefly, iPSN lysates were prepared by rinsing iPSNs with 1×PBS, adding mL supplied lysis buffer supplemented with DTT and Triton X-100 directly to each well, and harvesting iPSNs with a cell scraper. Postmortem brain and spinal cord lysates were prepared by homogenizing 150 mg fresh frozen tissue directly in supplied lysis buffer with a dounce homogenizer. All lysates were transferred to a 50 mL conical tube and vortexed. Sucrose gradients were assembled following manufacturer protocol. A 1.85 M sucrose gradient was used to enrich for neuronal nuclei. Samples were centrifuged at 15,600 rpm and 4° C. using a Swi32T swinging bucket rotor and Beckman ultracentrifuge (Beckman Coulter) for 45 minutes. The supernatant was discarded, and the remaining nuclei pellet was resuspended in 1 mL of supplied nuclei storage buffer to wash the nuclei of any remaining sucrose. Resuspended nuclei were centrifuged at 2500 rpm and 4° C. for 5 minutes. The supernatant was once again discarded, and the resulting nuclei pellet was vortexed in 1 mL of supplied nuclei storage buffer to resuspend for downstream imaging analysis. For western blots, washed nuclei were lysed in RIPA buffer as described below.

Super Resolution Structured Illumination Microscopy

Nuclei staining and super resolution imaging was performed as previously described (6). Following nuclei isolation, 10-50 μL of final nuclei/storage buffer suspension was centrifuged onto collagen coated (1 mg/mL; Advanced Biomatrix) slides with a CytoSpin 4 centrifuge (Thermo Fisher Scientific). Nuclei were immediately fixed with 4% PFA for 5 minutes, washed with 1×PBS 3×10 minutes and permeabilized with 1×PBST containing 0.1% Triton X-100 for 15 minutes. Nuclei were then blocked with 10% normal goat serum diluted in 1×PBS for 1 hour at room temperature and incubated in primary antibody diluted in block (10% normal goat serum in 1×PBS) overnight at 4° C. (See Table B for antibody information). After 16-18 hours, nuclei were washed with 1×PBS 3×10 minutes, incubated in secondary antibody diluted in block (10% normal goat serum in 1×PBS) for 1 hour at room temperature (See Table B for antibody information). Nuclei were washed with 1×PBS 3×10 minutes and cover slipped using Prolong Gold Antifade Reagent (Invitrogen) and 18 mm×18 mm 1.5 high tolerance coverslips (MatTek). NeuN or GFP positive nuclei (see Description of the Figures) were identified via microscope eye pieces. A single z section ˜110 nm thick was acquired by widefield imaging to confirm NeuN or GFP positivity. The immunostained Nup or ESCRT-III protein was subsequently imaged by super resolution structured illumination microscopy (SIM) using a Zeiss ELYRA 51. For each image, 5 grid rotations and optimal z sectioning parameters were employed. Following image acquisition, SIM images were reconstructed using default SIM processing parameters with Zeiss Zen Black 2.3 SP1 software. Automated nucleoporin spot and volume analysis was conducted as previously described (6, 26) using Imaris version 9.2.0 (Bitplane) and the 3D suite plugin in FIJI version 1.52p. Nucleoporin spots were counted using automated spot detection. A Bayesian classifier taking into account volume, average intensity, and contrast features was applied to detect and segment individual spots. The total number of nucleoporin spots was determined using a 3D-rendering of segmented SIM images comprising the entire depth of each nucleus.

When individual nucleoporin spots could not be resolved due to limits of resolution of immunofluorescent SIM, the percent total nuclear volume occupied by the nucleoporin was calculated. To calculate total nuclear volume, X and Y axis length was measured in the center z-slice for each nucleus and Z axis length was estimated from the total z depth of acquired images. To calculate nucleoporin volume, image stacks were processed with automatic thresholding and automatic thresholding the 3D suite plugin in FIJI was used to determine the volume of the thresholded area for each nucleus. Representative images are presented as 3D maximum intensity projections generated in Zeiss Zen Black 2.3 SP1. Images were faux colored green for contrast and display.

Human Tissue Immunofluorescence

Non-neurological control and C9orf72 patient postmortem paraffin embedded motor and occipital cortex sections were obtained through the Target ALS Human Postmortem Tissue Core (See Table C for demographic information).

TABLE C
Demographic Information

| | Clinical Diagnosis | Age of Death | Sex |
|---|---|---|---|
| Control-1 | Non-neurologic control | | Female |
| Control-2 | Non-neurologic control | 70 | Female |
| Control-3 | Non-neurologic control | 70 | Male |
| Control-4 | Non-neurologie control | 59 | Male |
| Control-5 | Non-neurologic control | 71 | Female |
| Control-6 | Non-neurologic control | 72 | Male |
| Control-7 | Non-neurologie control | 72 | Male |
| Control-8 | Non-neurologic control | 74 | Female |
| C9orf72-1 | C9orf72 | 72 | Male |
| C9orf72-2 | C9orf72 | 69 | Female |
| C9orf72-3 | C9orf72 | 61 | Female |
| C9orf72-4 | CSorf72 | 51 | Female |
| C9orf72-5 | C9orf72 | 59 | Male |
| C9orf72-6 | C9orf72 | 61 | Female |
| C9orf72-7 | C9orf72 | 68 | Female |

Tissue sections were gradually rehydrated with xylene 3×5 minutes, 100% ethanol 2×5 minutes, 90% ethanol 5 minutes, 70% ethanol 5 minutes, and finally dH2O 3×5 minutes. Antigen retrieval was performed with Tissue-Tek antigen retrieval solution (IHC World) for 1 hour in a steamer. Slides were cooled for 10 minutes and washed 3×5 minutes with dH2O, 2×5 minutes 1×PBS, and permeabilized with 0.4% Triton X-100 diluted in 1×PBS for 10 minutes on a shaker. Slides were subsequently washed 3×5 minutes in 1×PBS blocked with DAKO protein-free serum block (DAKO) overnight at 4° C. Tissue sections were incubated with primary antibody (See Table B for antibody information) diluted in DAKO antibody diluent reagent with background reducing components for a total of 2 overnights at 4° C. In between the 2 overnight incubations, slides were incubated in primary antibody at room temperature with gentle agitation for 10 hours. Following primary antibody, tissue sections were washed 3×5 minutes with 1×PBS and then incubated with secondary antibody (See Table B for antibody information) diluted in DAKO antibody diluent with background reducing components (DAKO) at room temperature with gentle agitation for 1 hour.

TABLE B
Antibody Information
Primary Antibodies

| | | Catalog | Application and |
|---|---|---|---|
| Antibody | Source | Number | Concentration |
| Mouse Anti-CHMP7 | Santa Cruz | sc-271805 | IF: 1/50 |
| | Biotechnology | | Western: 1/250 |
| Mouse Anti-VPS4 | Santa Cruz | sc-133122 | IF: 1/50 |
| | Biotechnology | | Western: 1/250 |
| Rabbit Anti-Nup50 | Abcam | ab137092 | IF: 1/250 |
| Rabbit Anti-POM121 | Novus Biologicals | NBP2-19890 | IF: 1/250 |
| Mouse Anti-Nup133 | Santa Cruz | Sc-376699 | IF: 1/50 |
| | Biotechnology | | |
| Mouse Anti-414 (FG | Abcam | Ab5000S | IF: 1/250 |
| Nups) | | | |
| Mouse Anti-CRM1 | Santa Cruz | sc-74454 | IF: 1/50 |
| (XPO1) | Biotechnology | | Western: 1/250 |
| Rabbit Anti-LEMD2 | Thermo Fisher | PA553589 | IF: 1/250 |
| | Scientific | | |
| Rabbit Anti-LEMD2 | Sigma Aldrich | HPA017340 | IF: 1/250 |
| | | | Western: 1/1000 |
| Chicken Anti-NeuN | Millipore | ABN91 | IF: 1/500 |
| Guinea Pig Anti-Map2 | Synaptic Systems | 188004 | IF: 1/1000 |
| Chicken Anti-GFP | Millipore | AB16901 | IF: 1/1000 |
| Mouse Anti-Turbo | Origene | TA150041 | IF: 1/250 |
| GFP | | | |
| Rabbit Anti-Turbo | Thermo Fisher | PA5-22688 | IF: 1/250 |
| GFP | Scientific | | |
| Rabbit Anti-DDX3X | Sigma Aldrich | HPA001648 | Western: 1/1000 |
| Rabbit Anti-NXP3 | Novus Biologicals | NBP2-19614 | Western: 1/1000 |
| Rabbit Anti-HuR | Cell Signaling | 12582S | Western: 1/1000 |
| (ELAVL1) | Technologies | | |
| Rabbit Anti-RanBP1 | Thermo Fisher | PA1080 | Western: 1/1000 |
| | Scientific | | |
| Rabbit Anti-Lamin B1 | Abcam | ab16048 | Western: 1/1000 |
| Rabbit Anti-Hsp90 | Abcam | ab13492 | Western: 1/1000 |
| Rabbit Anti-NXF1 | Abcam | ab129160 | Western: 1/1000 |
| Mouse Anti-GAPDH | Life Technologies | AM4300 | Western: |
| | | | 1/10,000 |

Secondary Antibodies

| | | | |
|---|---|---|---|
| Goat Anti-Mouse | Invitrogen | A11029 | IF: 1/1000 |
| Alexa 488 | | | |
| Goat Anti-Rabbit | Invitrogen | A11034 | IF: 1/1000 |
| Alexa 488 | | | |
| Goat Anti-Guinea Pig | Invitrogen | A11073 | IF: 1/1000 |
| Alexa 488 | | | |
| Goat Anti-Chicken | Invitrogen | A11039 | IF: 1/1000 |
| Alexa 458 | | | |
| Goat Anti-Mouse | Invitrogen | A11031 | IF: 1/1000 |
| Alexa 568 | | | |
| Goat Anti-Rabbit | Invitrogen | A11036 | IF: 1/1000 |
| Alexa 568 | | | |
| Goat Anti-Mouse | Invitrogen | A21236 | IF: 1/1000 |
| Alexa 647 | | | |
| Goat Anti-Rabbit | Invitrogen | A21245 | IF: 1/1000 |
| Alexa 647 | | | |
| Goat Anti-Chicken | Invitrogen | A21449 | IF: 1/1000 |
| Alexa 647 | | | |
| Donkey Anti-Rabbit | Thermo Fisher | 45-000-682 | Western: 1/5000 |
| IgQ HRP | Scientific | | |
| Horse Anti-Mouse IgG | Cell Signaling | 7076S | Western: 1/5000 |
| HRP | | | |

Slides were then washed 3×5 minutes in 1×PBS, rinsed briefly with 2-3 drops autofluorescence eliminator reagent (Millipore), and extensively washed 5×5 minutes in 1×PBS to remove debris. Tissue sections were stained with Hoescht diluted 1:1000 in 1×PBS for 20 minutes, and washed 3×5 mins in 1×PBS. Slides were coverslipped using Prolong Gold Antifade Reagent with DAPI and nuclei from Map2 positive Layer V neurons were imaged with a 20× objective and a Zeiss Axioimager Z2 fluorescent microscope equipped with an apotome2 module.

CHMP7 nuclear/cytoplasmic ratios were quantified using FIJI. Briefly, in focus neurons with visible nuclei were identified by positive Map2 and Hoescht signals. A small pixel box was used to measure fluorescent intensity in the CHMP7 channel. Two integrated density measurements were taken from each cellular compartment (nucleus and cytoplasm), the average of which was used for subsequent analysis. Five background fluorescent intensity mean measurements were taken in areas devoid of cells, the average of which was used for analysis. Nuclear and cytoplasmic intensities were calculated as follows: Intensity=Integrated Density−(Mean Background Intensity*Area Measured Within Cell). The nuclear/cytoplasmic CHMP7 ratio was calculated by dividing resulting nuclear intensity by cytoplasmic intensity for each cell. Images are presented as default apotome processed images generated in Zeiss Zen Blue 2.3.

Immunoprecipitation

XPO1: To couple antibody to beads, 504 Magnetic Dynabeads Protein G were rinsed and equilibrated in 1×PBST (0.02% Tween-20). PBST wash was removed from beads and discarded. Beads were then incubated with 10 μg Mouse Anti-XPO1 (Santa Cruz Biotechnology) or Mouse IgG Isotype control (Thermo Fisher Scientific) antibody diluted in 1×PBST (final volume 200 μL) with end over end rotation for 10 minutes at room temperature. The supernatant was discarded, and antibody coupled beads were washed with 200 μL 1×PBST. Antibody coupled beads were subsequently washed 2× with 200 μL conjugation buffer (20 mM NaPO₄, 0.15 M NaCl, pH 7.0). Antibody was then conjugated to the magnetic dynabeads by incubating in 250 μL 5 mM BS3 (Thermo Fisher Scientific) diluted in conjugation buffer with end over end rotation for 30 minutes at room temperature. The coupling reaction was quenched by adding 12.5 μL 1 M Tris HCl pH 7.5 and incubating with end over end rotation for 15 minutes at room temperature. Antibody conjugated beads were washed 3× with IP buffer (IP lysis buffer (Thermo Fisher Scientific), 1× protease inhibitor cocktail (Roche), 0.4 U/4 RNasin Plus (Promega)).

Nuclei were isolated from iPSNs as described above. The resulting nuclei pellet was lysed in 1 mL IP buffer by vortexing for 30-45 seconds. Nuclei lysates were centrifuged for 10 minutes at 2500 rpm and 4° C. to remove debris. Following centrifugation, the supernatant was transferred to a clean Eppendorf tube. 25 μL of the supernatant was added to μL 2× Laemmli buffer (BioRad) and set aside as protein input and an additional 25 μL supernatant was set aside on ice as RNA input. The remainder of the lysate was split equally between IgG and XPO1 antibody conjugated bead tubes. Lysates were incubated with beads with end over end rotation for 2 hours at 4° C. The unbound supernatant was discarded and immunoprecipitated complexes were washed 3×5 minutes in 200 μL IP buffer with end over end rotation at 4° C. Bead-IP complexes were resuspended in 200 μL IP buffer. 100 μL of each bead-IP complex sample was set aside for RNA analysis. For the remaining 100 μL, the supernatant was discarded and 50 μL 1× Laemmli buffer was added to bead-IP complexes. All RNA samples (including input) were added to 500 μL RLT Buffer (QIAGEN) and RNA isolation, cDNA synthesis, and qRT-PCR were performed as described below. Protein samples were analyzed by western blot as described below. Equal volumes of each sample (10 μL) were loaded in acrylamide gels.

NXF1: NXF1 immunoprecipitations were performed as described above without antibody-bead conjugation. Rabbit Anti-NXF1 (Abcam) and Rabbit IgG Isotype control (Thermo Fisher Scientific) antibodies were used.

Nuclear/Cytoplasmic Fractionation

iPSNs were rinsed in 1×PBS and nuclear/cytoplasmic fractionation was performed with the NE-PER Nuclear and Cytoplasmic Extraction Kit (Thermo Fisher Scientific) according to manufacturer protocol. Following the addition of C2 buffer, 25 μL of sample was added to 25 μL 2× Laemmli buffer (BioRad) and set aside as protein input. An additional 25 μL was set aside on ice as RNA input. From each nuclear and cytoplasmic fraction obtained, 25 μL sample was added to 25 μL 2× Laemmli buffer for protein analysis. An additional 25 μL was added to 500 μL RLT buffer (QIAGEN) for RNA analysis. The remainder of each fraction was stored at −80° C. At the completion of the fractionation, 500 μL RLT buffer was added to the RNA input sample. RNA isolation, cDNA synthesis, and qRT-PCR were performed as described below. Western blot analysis was conducted as described below. Equal volume of each sample (10 μL) was loaded in acrylamide gels.

qRT-PCR

RNA from immunoprecipitated complexes or nuclear and cytoplasmic cellular fractions was isolated with a RNeasy kit (QIAGEN) according to manufacturer protocol. RNA concentrations were determined with a NanoDrop 1000 spectrophotometer (Thermo Fisher Scientific). For detection of mRNA transcripts from immunoprecipitated complexes or nuclear and cytoplasmic cellular fractions, 1 μg of RNA was used for cDNA synthesis with random hexamers and the Superscript IV First-Strand cDNA Synthesis Kit (Thermo Fisher Scientific). qRT-PCR reactions were conducted using TaqMan Gene Expression Master Mix (Thermo Fisher Scientific), an Applied Biosystems Step One Plus Real Time PCR Machine (Thermo Fisher Scientific), and TaqMan Gene Expression Assays (See Table D, Thermo Fisher Scientific).

TABLE D
TaqMan probe IDs for qRT-PCR

| Target | TaqMan ID |
|---|---|
| 18S rRNA | Hs99999901_s1 |
| MAP1B | Hs01067016_m1 |
| ELAVL3 | Hs00154959_m1 |
| Beta Actin | Hs03023943_g1 |
| GAPDH | Hs02786624_g1 |

For detection of C9orf72 RNAs from immunoprecipitated complexes or nuclear and cytoplasmic cellular fractions, 1 μg of RNA was used for cDNA synthesis using gene specific primers and the Superscript IV First-Strand cDNA synthesis kit (Thermo Fisher Scientific). qRT-PCR reactions were performed out using TaqMan Gene Expression Master Mix (Thermo Fisher Scientific) and an Applied Biosystems Step One Plus Real Time PCR Machine (Thermo Fisher Scientific) and previously described primer/probe sets (6, 34) (See Table E for sequences). GAPDH was used for normalization in all experiments.

TABLE E
Primer and probe sequences for C9orf72 qRT-PCR

| Target | Sequence | |
|---|---|---|
| Human | Forward | TGTGACAGTTGGAATGCAGTGA |
| C9orf72 | Reverse | GCCACTTAAAGCAATCTCTGTCTTG |
| | Probe | TCGACTCTTTGCCCACCGCCA |
| G₄C₂ Repeat | Forward | GGGTCTAGCAAGAGCAGGTG |
| | Reverse | GTCTTGGCAACAGCTGGAGAT |
| | Probe | TGATGTCGACTCTTTGCCCACCGC |
| G₂C₄ Repeat | Forward | AGAAATGAGAGGGAAAGTAAAAATGC |
| | Reverse | CGACTGGAGCACGAGGACACTGACGGCTGCCGGGAAGA |
| | Probe | AGGAGAGCCCCCGCTTCTACCCG |

Human C9orf72 Forward Primer (Row 1) is SEQ ID NO: 5
Human C9orf72 Reverse Primer (Row 2) is SEQ ID NO: 6
Human C9orf72 Probe is (Row 3) SEQ ID NO: 7
G₄C₂ Repeat Forward Primer (Row 4) is SEQ ID NO: 8
G₄C₂ Repeat Reverse Primer (Row 5) is SEQ ID NO: 9
G₄C₂ Probe (Row 6) is SEQ ID NO: 10
G₄C₂ Repeat Forward Primer (Row 7) is SEQ ID NO: 11
G₄C₂ Repeat Reverse Primer (Row 8) is SEQ ID NO: 12
G₄C₂ Probe (Row 9) is SEQ ID NO: 13

Western Blots

Nuclei Lysates: Following nuclei isolation, nuclei pellets were resuspended in 25 μL RIPA buffer (Millipore) containing 1× protease inhibitor cocktail (Roche). Homogenates were spun at 12,000 g for 15 minutes and 4° C. to remove debris. The supernatant was transferred to a new Eppendorf tube and protein concentrations were determined using the BCA protein estimation assay kit (Thermo Fisher Scientific). 4× Laemmli buffer (BioRad) was added to each sample to a final concentration of 1×, samples were heated at 100° C. for 5 minutes, and 5 μg protein was loaded into 4-20% acrylamide gels (BioRad). Gels were run until the dye front reached the bottom. Protein was transferred onto a nitrocellulose membrane using the Trans-Blot Turbo Transfer System (BioRad). Blots were blocked for 30 minutes with 5% non-fat milk in 1×TBST (0.1% Tween-20) and incubated overnight at 4° C. with primary antibody diluted in block (See Table B for antibody information). The next day, blots were washed 4×10 minutes with 1×TBST and probed with secondary antibody diluted in block (See Table B for antibody information) for 1 hour at room temperature. Blots were subsequently washed 4×10 minutes with 1×TBST and ECL substrate (Thermo Fisher Scientific, Millipore) was applied for 30 seconds. Chemiluminescent images were acquired with the GE Healthcare ImageQuant LAS 4000 system. To sequentially probe membranes without stripping, chemiluminescent signals were quenched by incubating blots in room temperature 30% H₂O₂ for 15 minutes (35). Analysis was conducted in FIJI. Lamin B1 was used for normalization.

ASO Treated iPSN Lysates: On day 40 of differentiation, iPSNs were rinsed with ice cold 1×DPBS with Ca²⁺ and Mg²⁺. 1 mL fresh 1×DPBS was added to each well and iPSNs were harvested with a cell scraper and transferred to an Eppendorf tube. Cells were then pelleted at 2500 rpm, 4° C. for 5 minutes. The supernatant was aspirated, and the resulting cell pellet was lysed in 50 μL RIPA buffer containing 1× protease inhibitor cocktail. Western blots and analysis were performed as described above. GAPDH was used for normalization.

Trim Away Samples: Following aspiration of media, iPSNs were rinsed with ice cold 1×DPBS with Ca²⁺ and Mg²⁺. 1 mL fresh 1×DPBS was added to each well and iPSNs were harvested with a cell scraper and transferred to an Eppendorf tube. Samples were centrifuged at 2500 rpm, 4° C. for 5 minutes to pellet cells. The supernatant was aspirated, and the resulting cell pellet was lysed in 25 μL RIPA buffer containing 1× protease inhibitor cocktail. Western blots and analysis were performed as described above. GAPDH was used for normalization.

Plasmids and Nucleofection for CHMP7 and VPS4 Overexpression

The GFP tagged CHMP7 plasmid was obtained from Origene. To generate the CHMP7 NES mutant (CHMP7 NES*) plasmid, a single amino acid substitution (amino acid 430, L to A) was created in the CHMP7 GFP plasmid (Origene) using the Q5 site directed mutagenesis kit (New England BioLabs). VPS4 GFP and VPS4^{E228Q} GFP plasmid were obtained from Addgene. GFP control plasmids were obtained from Origene and Addgene. RNA Only plasmids (12) were a kind gift from Adrian Issacs. See Table F for plasmid information.

TABLE F
Plasmid information

| Plasmid | Backbone | Catalog Number |
|---|---|---|
| CHMP7 GFP | pCMV6-AC-GFP | Origene RG222461 |
| CHMP7 NES* GFP | pCMV6-AC-GFP | N/A, this study |
| VPS4 GFP | pLNCX2 | Addgene 116924 |
| VPS4^{E228Q} GFP | pEGFP-C1 | Addgene 80351 |
| GFP | pCMV6-AC-GFP | Origene PS100010 |
| GFP | mEGFP-C1 | Addgene 54759 |
| 36RO | pcDNA3.1+ | N/A, Mizielinska et al., 2014 |
| 106RO | pcDNA3.1+ | N/A, Mizielinska et al., 2014 |
| 288RO | pcDNA3.1+ | N/A, Mizielinska et al., 2014 |
| Trim21 GFP | pCMV6-AC-GFP | Origene RG202088 |

On day 18 of differentiation, iPSNs were dissociated with accutase to assist with single cell dissociation and nucleofected in suspension using the Lonza P3 Primary Cell 4D Nucleofector Kit (Lonza) and program DC104 on the Lonza nucleofection system. Each cuvette contained 5×106 iPSNs and 4 μg plasmid DNA. Following nucleofection, iPSNs were plated in Matrigel (Corning) coated cell culture dishes according to Lonza protocol. Media was exchanged on day 19 and 22 of differentiation. Downstream experimentation was carried out on day 25 of differentiation.

Knockdown of CHMP7, LEMD2, and XPO1 by Trim Away

Knockdown of endogenous CHMP7, LEMD2, and XPO1 was carried out using a modified Trim Away protocol (11). CHMP7 (Santa Cruz Biotechnology), LEMD2 (Thermo Fisher Scientific), and XPO1 (Santa Cruz Biotechnology) antibodies were dialyzed in 1×PBS using a Slide-A-Lyzer MINI Dialysis Device, 20K MWCO (Thermo Fisher Scientific). PBS was exchanged after 2 hours and dialysis proceeded with gentle agitation at room temperature overnight. Dialyzed antibodies were concentrated with an Amicon Ulta-0.5 Centrifugal Filter Unit with Ultacell-100 membrane (Millipore). Resulting antibody concentration was calculated with a NanoDrop 1000 spectrophotometer (Thermo Fisher Scientific) as previously described (11). On day 18 of differentiation, iPSNs were dissociated with accutase. Suspension based nucleofection was carried out with the Lonza P3 Primary Cell 4D Nucleofector Kit (Lonza) and the program DC154. Each cuvette contained 5×10 6 iPSNs, 5 μg of antibody and 4 μg Trim21 GFP plasmid DNA (Origene, See Table F). Following nucleofection, iPSNs were replated in Matrigel (Corning) coated cell culture dishes according to Lonza protocol and used for downstream analysis on day 20 of differentiation.

Glutamate Toxicity

On day 12 of differentiation, iPSNs were plated in 24 well optical bottom plates (Cellvis) at a density of 250,000 neurons per well. Neurons were rinsed with 1×PBS and fed with fresh stage 3 media daily remove dead cells and debris until day 25 of differentiation. On day 25 of differentiation, ASO treatment was initiated as described above. Every 3 days, iPSNs were rinsed 3× with 1×PBS and media and ASO were replaced. On day 40 of differentiation, iPSNs were washed with 1×PBS to remove any remaining debris and dead cells. Media was replaced with ACSF (Tocris) containing 0 or 10 μM glutamate (Sigma Aldrich). iPSNs were incubated at 37° C. with 5% CO₂ for 4 hours. After 3.5 hours, one drop of NucBlue Live ReadyProbes (Thermo Fisher Scientific) and 1 propidium iodide (Thermo Fisher Scientific) and returned to the incubator for 30 mins. iPSNs were imaged in an environmentally controlled chamber with a Zeiss LSM 800 confocal microscope. 5 images per well were acquired with a 10× objective. PI and DAPI spots were counted using FIJI.

### Statistical Analysis

All data analysis was conducted with Imaris or FIJI as described in each experimental section above and was either completely automated or blinded. All statistical analyses were performed using GraphPad Prism version 8 (GraphPad). For imaging experiments where multiple cells or nuclei per iPSC line or patient were quantified, statistical analyses were performed such that the average of all nuclei or cells evaluated per iPSC line or patient represents N=1 with total N per experiment and group as indicated in Description of the Figures. Student's t-test, One-way ANOVA with Tukey's multiple comparison test, or Two-way ANOVA with Tukey's multiple comparison test was used as described in the Description of the Figures. * p<0.05, ** p<0.005, *** p<0.0005, **** p<0.0001. Violin plots are used to display the full spread and variability of large data sets (>10 data points). Center dotted line indicates median value. Two additional dotted lines indicate the 25th and 75th percentiles. For smaller data sets, bar graphs displaying individual data points shown where error bars represent +/−SEM.

### Example 2—Nuclear Expression of CHMP7 and VPS4 is Increased in C9orf72 iPSNs and Postmortem Human Brain as a Result of G₄C₂ Repeat RNA

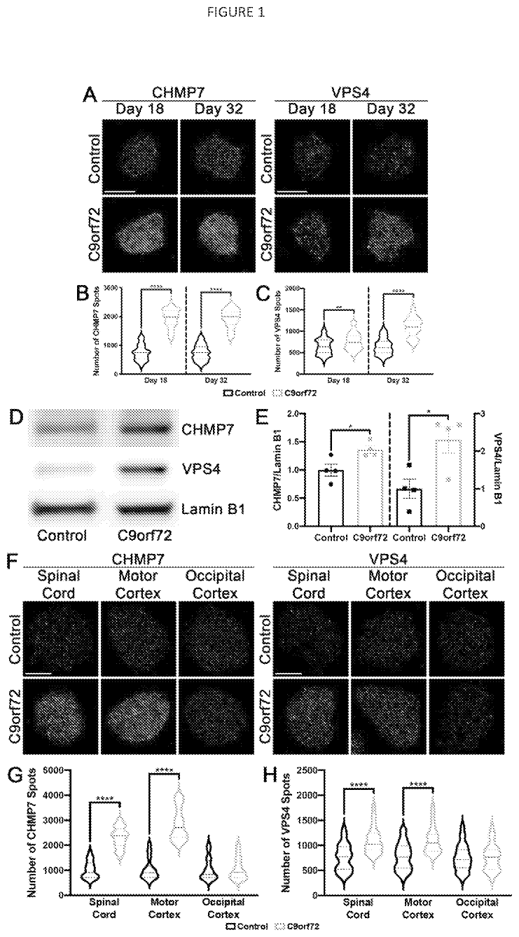

Recently, it was established that the protein expression of eight specific Nups is reduced within the nucleoplasm and NPCs of C9orf72 human neurons (13). Given the critical involvement of CHMP7 and VPS4 in NPC homeostasis in yeast (7-9), it was investigated whether the expression of these proteins was pathologically altered in C9orf72 nuclei. Using super resolution structured illumination microscopy (SIM) to visually examine nuclear distribution and expression, we found a significant increase in both CHMP7 and VPS4 immunoreactivity in NeuN positive nuclei isolated from control and C9orf72 iPSNs (FIGS. 1A-C). Interestingly, this increase was observed even at a time point (Day 18 of differentiation, FIG. 1A-1C) preceding that at which Nup reduction was previously observed (6). This suggest that CHMP7 is not responding to NPC injury but may play a more direct role in its initiation. Additionally, western blot analyses quantitatively confirmed an increase in CHMP7 and VPS4 protein levels in C9orf72 nuclei (FIGS. 1D-1E).

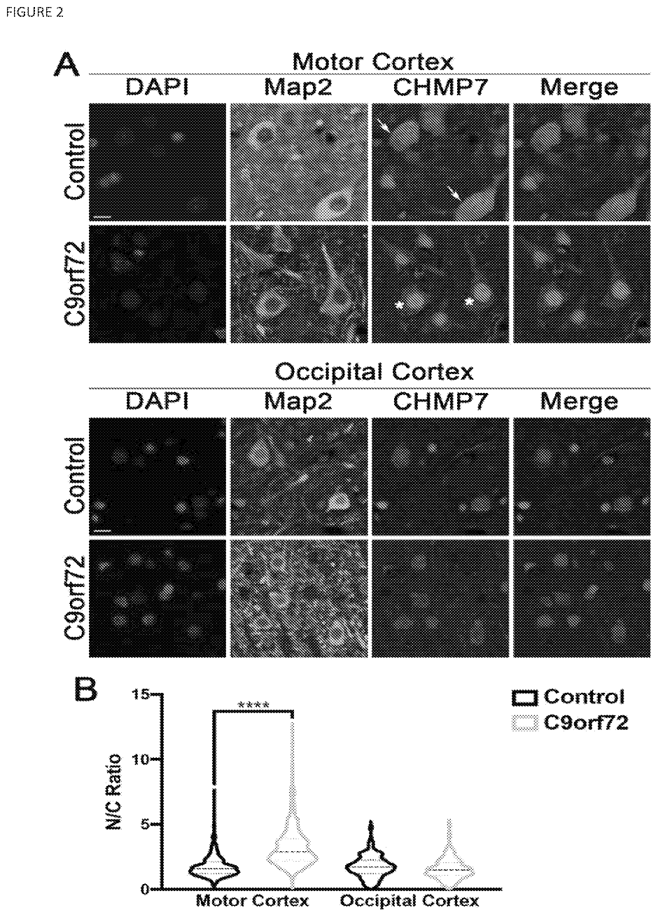

To verify that our result in iPSNs recapitulated human disease pathology, nuclei were isolated from control and C9orf72 postmortem spinal cord, and motor and occipital cortex tissue and performed SIM for CHMP7 and VPS4. In the disease relevant CNS regions (thoracic spinal cord and motor cortex), but not the unaffected occipital cortex, a similar increase was observed in the nuclear expression of CHMP7 and VPS4 (FIGS. 1F-1H). Moreover, immunofluorescent staining in thin sections reveals a significant relocalization of CHMP7 to the nucleus in Map2 positive neurons in postmortem patient motor but not occipital cortex (FIG. 2).

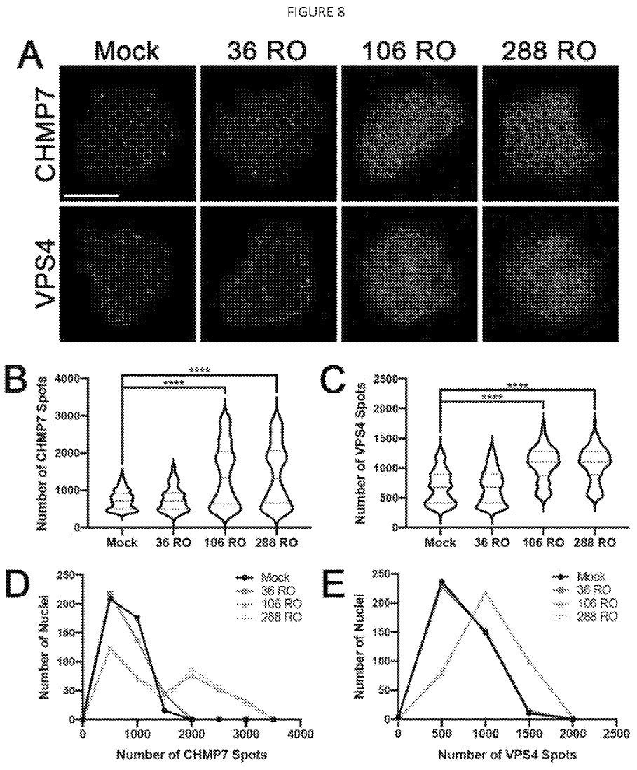

Nup alterations are known to be a pathological consequence of expanded G₄C₂ repeat RNA but not DPRs or loss of C9ORF72 protein (6). To determine if the nuclear increase in CHMP7 and VPS4 was similarly mediated by G₄C₂ repeat RNA, we performed SIM on nuclei isolated from control iPSNs overexpressing stop codon optimized constructs that only produce G₄C₂ repeat RNA (12). Overexpression of 106 or 288, but not 36 G₄C₂ repeats resulted in a significant increase in nuclear CHMP7 and VPS4 immunoreactivity (FIG. 8). Collectively, these data suggest that the nuclear localization of CHMP7 and VPS4 are pathologically increased in C9orf72 neuronal nuclei as a result of expanded G₄C₂ repeat RNA.

### Example 3—Knockdown of CHMP7 Protein Restores the Nuclear Expression of Specific Nups in C9orf72 iPSNs

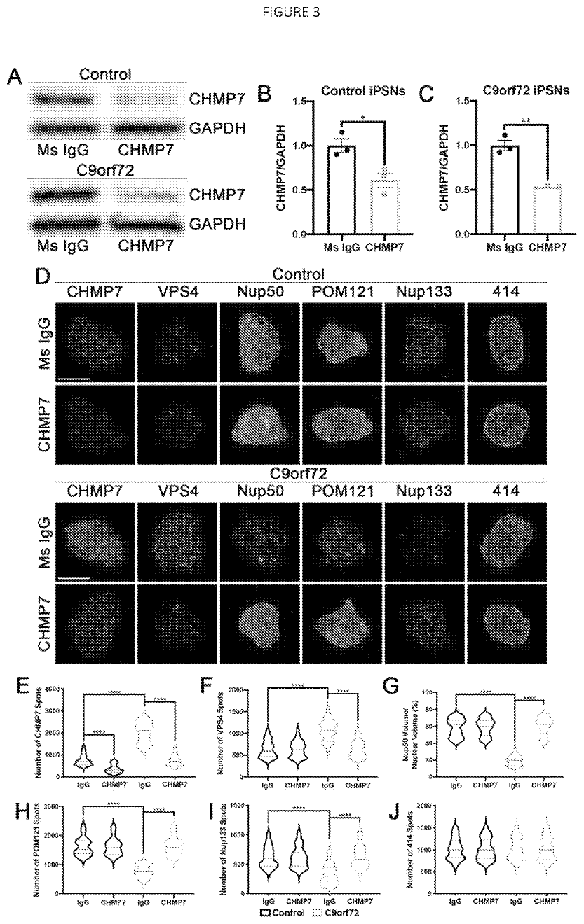

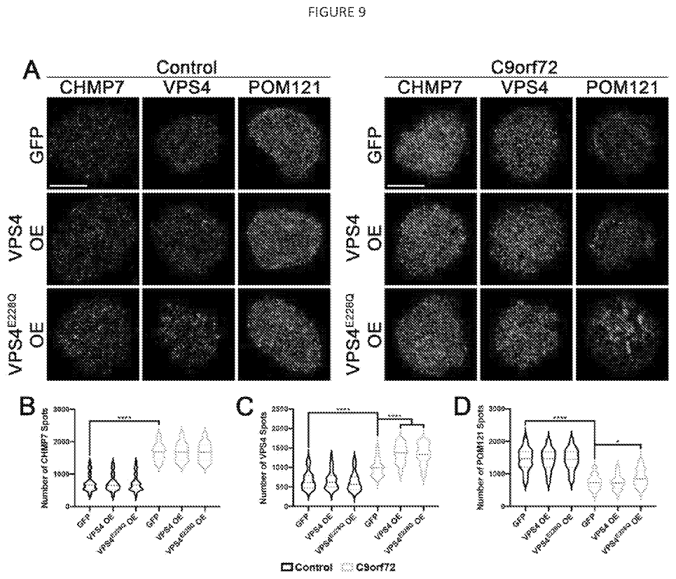

As CHMP7 has previously been implicated as an early-acting factor that mediates Nup and NPC degradation (7-9), we hypothesized that perhaps the nuclear increase in CHMP7 might lead to reduced Nup expression in C9orf72 neuronal nuclei and NPCs. To test this, the Trim Away method (11) was employed to rapidly degrade endogenous CHMP7 protein in control and C9orf72 iPSNs (FIGS. 3A-3C). Using SIM, it was found that Trim21 GFP mediated knockdown of CHMP7 (FIGS. 3D-E) significantly mitigated the reduction in Nup50, POM121, and Nup133 immunoreactivity in C9orf72 iPSN nuclei (FIGS. 3D, 3G-3I). Importantly, knockdown of CHMP7 had no effect on FG repeat containing Nups as assayed by mAb414 staining (FIGS. 3D, 3J) suggesting that increased nuclear CHMP7 may only impact the nuclear levels of a subset of Nups in human neurons. Moreover, VPS4, which generally acts at a terminal step in ESCRT-mediated cellular processes (13), is reduced in C9orf72 iPSN nuclei upon CHMP7 knockdown (FIGS. 3D, 3F). Consistent with a function for VPS4 downstream of CHMP7, the overexpression of a GFP tagged dominant negative variant of VPS4 (VPS4^{E228Q}, FIGS. 9A, 9C) only partially restored the nuclear expression of POM121 (FIGS. 9A, 9D), and had no effect on increased nuclear CHMP7 in C9orf72 iPSN nuclei (FIGS. 9A-B). Interestingly, the distribution of POM121 appears to be abnormal in C9orf72 nuclei overexpressing VPS4^{E228Q} (FIG. 9A). Together, these data suggest that increased nuclear CHMP7 can initiate a pathological cascade impacting the nuclear levels of specific Nups spanning multiple subcomplexes of the neuronal NPC within C9orf72 nuclei.

### Example 4—ASO Mediated Knockdown of CHMP7 Mitigates Nup Alterations and Improves Neuronal Survival

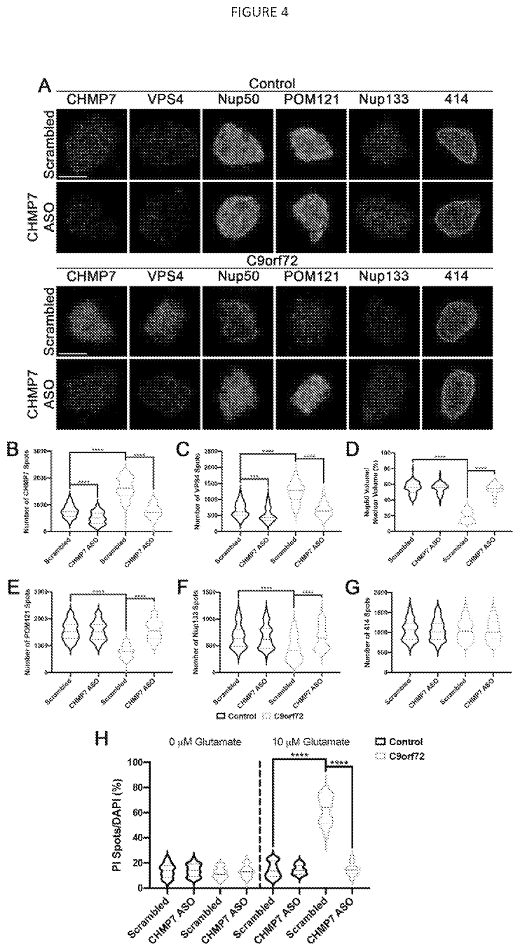

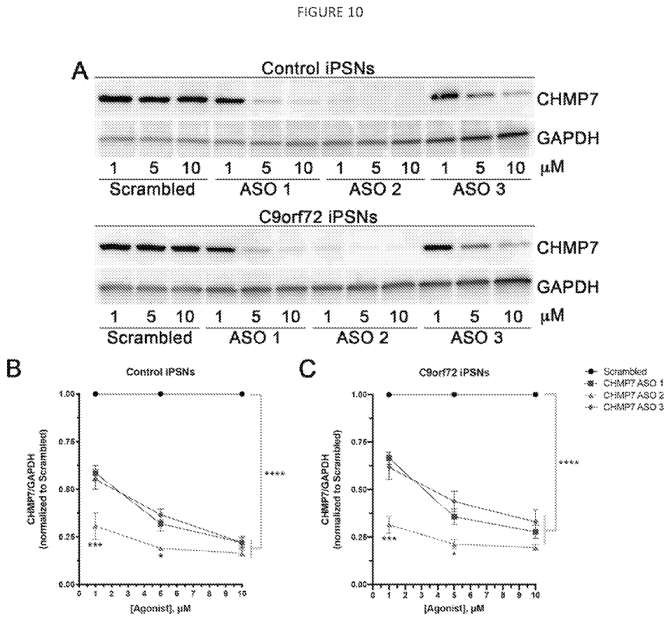

ASOs are now commonly used as a therapeutic strategy to reduce mRNA and protein levels of genes of interests (14, 15). To evaluate if this therapeutic strategy would effectively mitigate NPC injury, ASOs were designed that specifically target human CHMP7 pre-mRNA and induce its degradation via RNase H based mechanisms. Compared to scrambled control ASOs, 2-week exposure to 3 different CHMP7 targeting ASOs, significantly reduced CHMP7 protein levels in control and C9orf72 iPSNs in a dose dependent manner (FIG. 10). Consistent with our Trim Away experiments (FIG. 3), CHMP7 ASO treatment significantly reduced nuclear levels of both ESCRT-III proteins CHMP7 and VPS4 in control and C9orf72 iPSNs (FIGS. 4A-C). Moreover, compared to a scrambled control ASO, 5 μM CHMP7 ASO 2 significantly restored POM121, Nup133, and Nup50 expression in C9orf72 iPSN nuclei and NPCs (FIGS. 4A, 4D-4F) as evaluated by SIM. Importantly, no disruption in Nups was observed in control iPSNs treated with CHMP7 ASOs (FIGS. 4A, 4D-4G) nor on FG Nups in C9orf72 iPSNs (FIGS. 4A, 4G) suggesting that ASO mediated knockdown in CHMP7 protein levels specifically mitigates disease associated Nup alterations in iPSNs. Moreover, CHMP7 ASOs significantly decreased the sensitivity of C9orf72 iPSNs to glutamate induced excitotoxicity (FIG. 4H) as measured by propidium iodide (PI) incorporation as previously described (6, 16). Collectively, these data suggest that CHMP7 ASOs can effectively alleviate Nup defects and downstream neuronal death in C9orf72 iPSNs.

### Example 5—LEMD2 does not Mediate Increased Nuclear CHMP7 in C9orf72 iPSNs

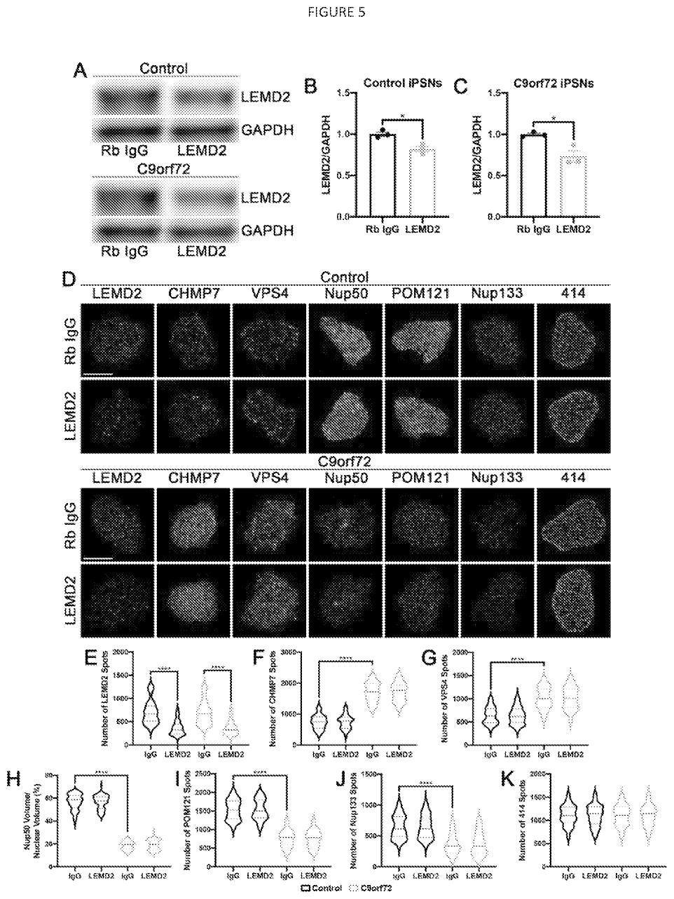

Having established that increased nuclear CHMP7 can impact the nuclear expression of VPS4 and specific Nups, it was next sought to determine the mechanism by which this pathology occurred in C9orf72 nuclei. As both the yeast and human CHMP7 orthologues are recruited to the nuclear envelope by the integral INM protein LEMD2 (9, 17), the Trim Away method was used to endogenously knockdown LEMD2 in control and C9orf72 iPSNs (FIGS. 5A-C). In contrast to knockdown of CHMP7, it was found that a small but significant reduction in nuclear LEMD2 has no effect on the nuclear immunoreactivity of CHMP7, VPS4, or specific Nups using SIM (FIGS. 5D-5K). Thus, this data suggests that LEMD2 mediated recruitment of CHMP7 to the nuclear envelope is not responsible for increased nuclear expression of CHMP7 in C9orf72 iPSNs.

### Example 6—Inhibition of CHMP7 Nuclear Export Recapitulates C9orf72 Mediated Reduction in Specific Nucleoporins

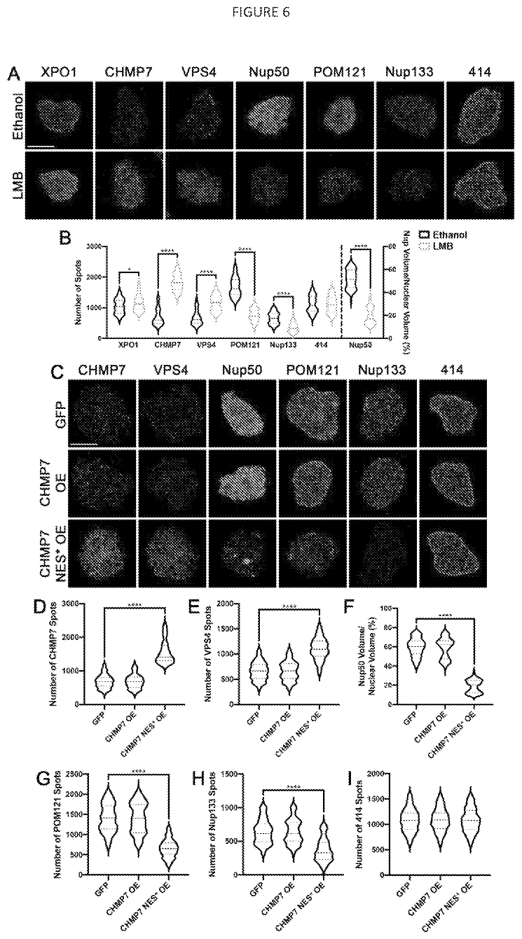

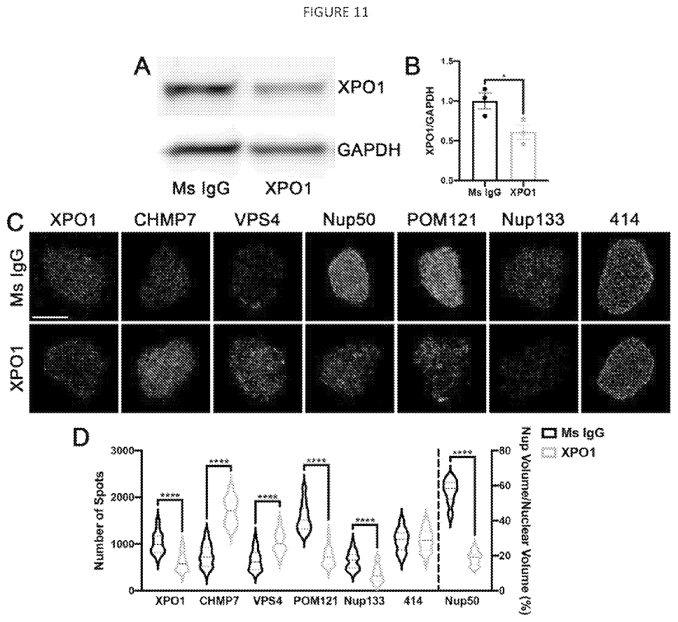

In unperturbed conditions, both yeast and human CHMP7 cannot accumulate in the nucleus due to its nuclear export by XPO1/CRM1 (8, 18). Therefore, Leptomycin B (LMB) was used to potently inhibit global XPO1 function (19) and performed SIM for CHMP7, VPS4, and specific nucleoporins. Consistent with published data, (8, 18) LMB treatment resulted in a significant increase in CHMP7 in the nucleus (FIGS. 6A-6B). Remarkably, this occurred alongside an increase in VPS4 and a concomitant reduction in Nup50, POM121, and Nup133, but not FG-Nup immunoreactivity in nuclei isolated from wildtype iPSNs (FIGS. 6A-6B). These results were also recapitulated by a direction reduction of XPO1 levels using Trim Away (FIG. 11). Thus, by globally inhibiting XPO1 mediated nuclear export, disease phenotypes could be recapitulated in wildtype human neurons.

To determine whether the reduction in Nup levels in LMB treated iPSNs was a direct result of specifically inhibiting CHMP7 nuclear export, the nuclear export sequence (NES) of CHMP7 was mutated by making a single amino acid substitution (L to A, amino acid 430) in the CHMP7 NES sequence (LEAELEKLSLS (SEQ ID NO:21)). As previously described (8), this single point mutation is sufficient to abrogate XPO1 mediated nuclear export of CHMP7 (34). Overexpression of the GFP tagged CHMP7 NES mutant (CHMP7 NES*), but not wildtype GFP tagged CHMP7, in wildtype iPSNs recapitulated C9orf72 mediated alterations in nuclear CHMP7, VPS4, Nup50, POM121, and Nup133 levels as evaluated by SIM (FIG. 6C-6I). Collectively, this data suggests that aberrant nuclear accumulation of CHMP7 is sufficient to facilitate alterations to Nup levels in C9orf72 iPSN nuclei.

### Example 7—G4C2 Repeat RNA Reduces the Presence of CHMP7 in Nuclear XPO1 Complexes

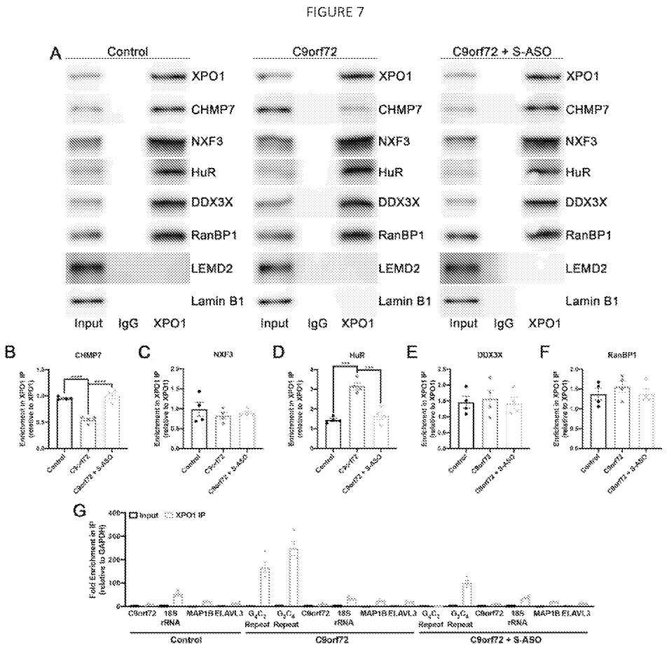

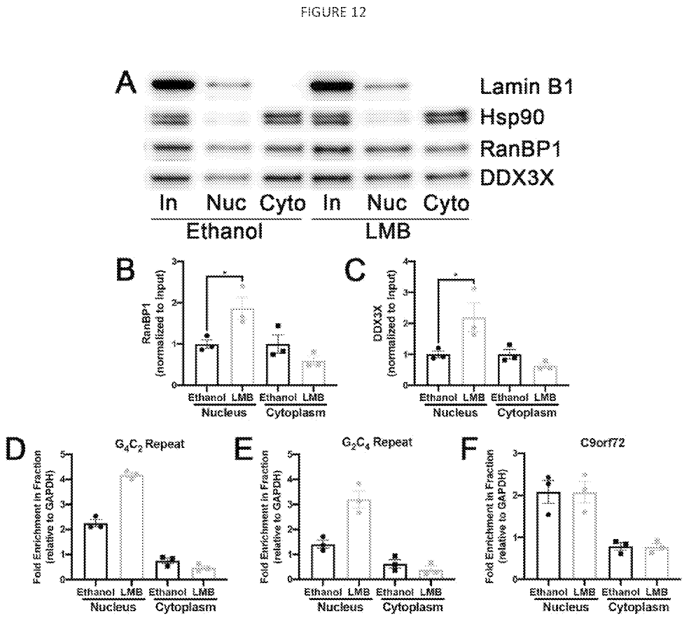

The data establishes that expression of pathologic G4C2 repeat RNA results in increased nuclear CHMP7 and VPS4 levels (FIG. 8) and that impaired nuclear export of CHMP7 in wildtype iPSNs can recapitulate C9orf72 mediated alterations in specific Nups (FIG. 6). Therefore, it was hypothesized that perhaps G4C2 repeat RNA might disrupt the association between CHMP7 and XPO1, which would result in the nuclear accumulation of CHMP7. To test this, co-immunoprecipitation was performed experiments to examine the proportion of nuclear protein and RNA pools present within XPO1 complexes in human neurons. Western blot analyses confirmed that we efficiently immunoprecipitated nuclear associated XPO1 complexes that contain the known XPO1 interacting proteins NXF3 and HuR (21-23) (FIGS. 7A, 7C-7D). Although devoid of LEMD2 and Lamin B1 (FIG. 7A), these immunoprecipitated complexes contain DDX3X and RanBP1 (FIGS. 7A, 7E-7F). Both DDX3X and RanBP1 contain predicted NESs and LMB treatment increases their nuclear levels in non-neuronal cell lines (24) and iPSNs (FIGS. 12A-12C), suggesting that both proteins are XPO1 cargoes. Interestingly, the association of nuclear pools of these specific cargo proteins with XPO1 complexes is not disrupted in the context of the C9orf72 HRE (FIGS. 7A, 7E-7F). In contrast, there was a significant decrease in the association of nuclear pools of CHMP7 with immunoprecipitated XPO1 complexes (FIGS. 7A-7B) in C9orf72 iPSN nuclei compared to controls.

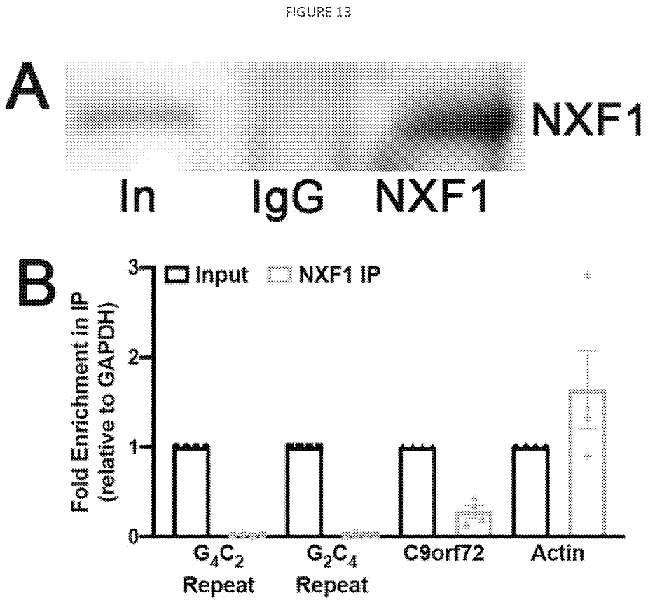

Using qRT-PCR, we found that both the pathologic G4C2 and G2C4 repeat RNAs are also present in a complex with XPO1 (FIG. 7G) but not NXF1 (FIG. 13), a key component of the canonical NXF-NXT1 mRNA export pathway (21-23). Notably the association of G4C2 and G2C4 repeat RNAs with XPO1 complexes was increased compared to that of RNAs (18S rRNA, and ARE sequence containing RNAs MAP1B and ELAVL3) whose nuclear export may at least in part be governed by XPO1 (35-37) (FIG. 7G).

It has been previously established that five day treatment with antisense oligonucleotides (ASOs) targeting the intron upstream of the sense strand G4C2 repeat decreases G4C2 repeat RNA but not DPR levels and mitigates alterations in POM121 (6). As a result, it was evaluated whether a reduction in G4C2 repeat RNA could impact the association of CHMP7 with XPO1 complexes. Following short term ASO treatment, we observed a reduction in the presence of both G4C2 and G2C4 repeat RNAs in XPO1 complexes (FIG. 7G). Moreover, western blot analyses revealed a restored association of CHMP7 with nuclear XPO1 complexes (FIGS. 7A-B). Collectively, these data suggest that G4C2 repeat RNA may specifically disrupt the association of CHMP7 with XPO1 complexes without disrupting global cargo export to turn initiate Nup alterations in the pathogenesis of C9orf72 ALS/FTD.

Pathological Nup alterations are prevalent in multiple neurodegenerative diseases including ALS, AD, and HD (1, 25-27). However, the molecular mechanisms by which these disruptions occur remain poorly understood. In contrast to the Nup mislocalization observed in artificial overexpression mouse models of C9orf72 ALS/FTD (2-5), it was previously shown that in human neurons, there is a reduction in the nuclear levels of 8 specific Nups spanning multiple subcomplexes of the NPC without corresponding cytoplasmic accumulations (6). As a result, it was hypothesized that altered Nup surveillance and homeostasis may contribute to reduced Nup expression in C9orf72 iPSNs.

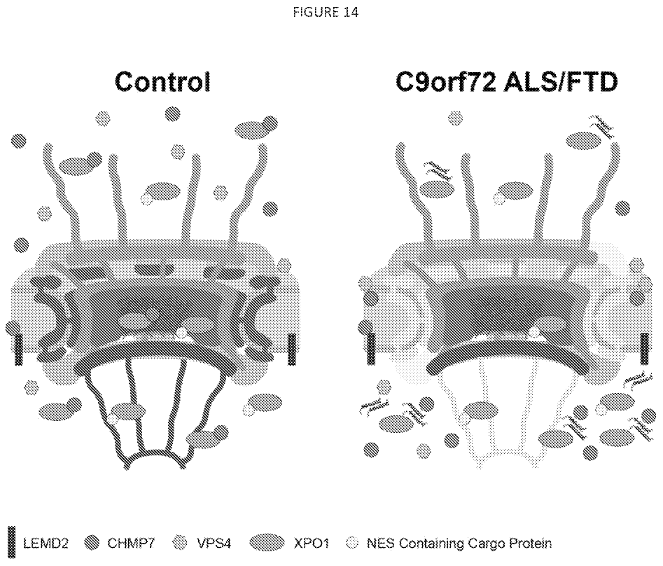

Recent work in yeast and non-neuronal cellular systems has identified CHMP7 and ESCRT-III mediated degradation as important mediators of NPC, Nup, and nuclear envelope homeostasis (7, 9, 10, 28). However, the contribution of this pathway to neuronal biology and disease pathophysiology remains unclear. Using an iPSN model of C9orf72 ALS, it was found that CHMP7 contributes to reduced nuclear levels of key Nups located within 3 distinct domains of the NPC as an early event in disease pathogenesis. These data define a functional role for CHMP7 and ESCRT-III mediated Nup proteostasis in human neurons and neurodegeneration. Collectively, our data support a model whereby pathologic G4C2 repeat RNA interferes with the association between CHMP7 and XPO1 (see FIG. 14). Notably, the association of other NES containing cargo proteins DDX3X and RanBP1 with XPO1 is unaltered in C9orf72 nuclei suggesting that expanded G4C2 repeat RNA may affect the nuclear export of specific, but not global, XPO1 cargoes. The specific inhibition of CHMP7 nuclear export may in turn initiate a downstream pathological cascade whereby the subsequent recruitment of VPS4 mediates a reduction in the nuclear levels of specific Nups (see FIG. 14) in human neurons. Future experiments are necessary to determine whether endogenous G₄C₂ repeat RNA binds directly to specific domains of CHMP7 and/or XPO1 to disrupt this specific protein-protein interaction without overt disruption to global XPO1 cargo association.

Previous studies have suggested that the knockdown of NXT1 increases nuclear G₄C₂ repeat RNA levels in artificial overexpression non-CNS cellular systems (29). Interestingly, while NXT1 is a critical component of the NXF1-NXT1 nuclear RNA export pathway (21-23), NXT1 has also been shown to associate with CRM1/XPO1 (44). Here it was shown that in human iPSNs expressing endogenous levels of the C9orf72 HRE, G4C2 and G₂C₄ repeat RNAs strongly associate with XPO1 but not NXF1 nuclear complexes (FIGS. 7G, FIG. 13). Moreover, treatment of C9orf72 iPSNs with the XPO1 inhibitor, LMB, strongly increases the presence of G₄C₂ and G₂C₄ repeat RNAs in the nuclear fraction (FIGS. 12D-12E). Together, these data suggest that the nuclear export of G₄C₂ and G₂C₄ repeat RNAs may at least be in part mediated by XPO1 complexes in human neurons. Notably, it has been shown that while the NXF1-NXT1 pathway is the primary mRNA export pathway in multiple cell types, XPO1, in combination with different “adapter” proteins, can function in the export of various RNA species including some specific intron containing mRNAs (21, 23).

Although ESCRT-III mediated NPC and nuclear envelope repair is initiated by the nuclear specific ESCRT adapter protein CHMP7 (31) and catalyzed by VPS4 (7-9, 13, 31, 32), the recruitment and involvement of other CHMPs in this process remains unknown in multiple cellular systems including human neurons. Given that mutations in the ESCRT-III pathway component CHMP2B have been implicated in FTD (33), it will be interesting to evaluate whether or not these mutations are sufficient to induce nucleoporin reduction in a manner similar to that observed in C9orf72 ALS/FTD iPSNs.

Collectively, the present data suggest that increased nuclear CHMP7 is a critical initiator of pathogenic cascades ultimately leading to Nup reduction in the nucleoplasm and NPCs of C9orf72 neurons. Moreover, the present data highlight CHMP7 as a potential therapeutic target for the mitigation of NPC injury in neurodegenerative diseases characterized by nuclear Nup reduction.

### Example 8

The below materials and methods were used for Examples 9-15.

iPSC Derived Neuronal Differentiation

C9orf72 and non-neurological control iPSC lines were obtained from the Answer ALS repository at Cedars-Sinai (see Table G for demographics). Feeder-free iPSCs were maintained on Matrigel with MTeSR and maintained according to Cedars Sinai SOP. iPSCs were differentiated into spinal neurons using the previously described direct induced motor neuron (diMNs) protocol (43). All cells were maintained at 37° C. with 5% CO₂. iPSCs and iPSNs routinely tested negative for mycoplasma.

TABLE G
Demographic Information for iPSC Lines

| iPSC Line | | Clinical | Age at Time | | |
|---|---|---|---|---|---|
| Name | Source | Diagnosis | of Collection | Sex | Origin |
| EDi036-A | Cedars- | Non-neurologic | 79 | Female | PBMC |
| | Sinai | control | | | |
| EDi037-A | Cedars- | Non-neurologic | 79 | Male | PBMC |
| | Sinai | control | | | |
| EDi029-A | Cedars- | Non-neurologic | 80 | Male | PBMC |
| | Sinai | control | | | |
| EDi034-A | Cedars- | Non-neurologic | 79 | Female | PBMC |
| | Sinai | control | | | |
| EDi022-A | Cedars- | Non-neurologic | 79 | Male | PBMC |
| | Sinai | control | | | |
| EDi044-A | Cedars- | Non-neurologic | 80 | Female | PBMC |
| | Sinai | control | | | |
| CS1ATZ | Cedars- | Non-neurologic | 60 | Male | PBMC |
| | Sinai | control | | | |
| CS8PAA | Cedars- | Non-neurologic | 58 | Female | PBMC |
| | Sinai | control | | | |
| EDi043-A | Cedars- | Non-neurologic | 80 | Male | PBMC |
| | Sinai | control | | | |
| CS0201 | Cedars- | Non-neurologic | 56 | Female | PBMC |
| | Sinai | control | | | |
| CS0002 | Cedars- | Non-neurologic | 51 | Male | PBMC |
| | Sinai | control | | | |
| CS9XH7 | Cedars- | Non-neurologic | 53 | Male | PBMC |
| | Sinai | control | | | |
| CS0206 | Cedars- | Non-neurologic | 72 | Female | PBMC |
| | Sinai | control | | | |
| CS0202 | Cedars- | Non-neurologic | 57 | Male | PBMC |
| | Sinai | control | | | |
| CS2AE8 | Cedars- | Non-neurologic | 50 | Female | PBMC |
| | Sinai | control | | | |
| CS9BP3 | Cedars- | Non-neurologic | 48 | Female | PBMC |
| | Sinai | control | | | |
| CS1FER | Cedars- | Non-neurologic | 62 | Male | PBMC |
| | Sinai | control | | | |
| CS0BUU | Cedars- | C9orf72 | 63 | Female | PBMC |
| | Sinai | | | | |
| CS7VCZ | Cedars- | C9orf72 | 64 | Male | PBMC |
| | Sinai | | | | |
| CS0LPK | Cedars- | C9orf72 | 67 | Male | PBMC |
| | Sinai | | | | |
| CS6ZLD | Cedars- | C9orf72 | | Female | PBMC |
| | Sinai | | | | |
| CS8KT3 | Cedars- | C9orf72 | 60 | Male | PBMC |
| | Sinai | | | | |
| CS2YNL | Cedars- | C9orf72 | 60 | Male | PBMC |
| | Sinai | | | | |
| CS0NKC | Cedars- | C9orf72 | 52 | Female | PBMC |
| | Sinai | | | | |
| CS6CLW | Cedars- | C9orf72 | | Male | PBMC |
| | Sinai | | | | |
| 59-1 | K. Talbot | Isogenic | 62 | Female | Fibro- |
| | | Correction of | | | blast |
| | | OXC9-02 | | | |
| OXC9-02- | K. Talbot | C9orf72 | 62 | Female | Fibro- |
| 02 | | | | | blast |
| CS0JGZ | Cedars- | sALS | 56 | Male | PBMC |
| | Sinai | | | | |
| CS2EVP | Cedars- | sALS | 69 | Male | PBMC |
| | Sinai | | | | |
| CS3XLK | Cedars- | sALS | 55 | Female | PBMC |
| | Sinai | | | | |
| CS5JPF | Cedars- | sALS | 55 | Female | PBMC |
| | Sinai | | | | |
| CS4ZCD | Cedars- | sALS | 53 | Male | PBMC |
| | Sinai | | | | |
| CS8JGP | Cedars- | sALS | 61 | Male | PBMC |
| | Sinai | | | | |
| CS6MBU | Cedars- | sALS | | Male | PBMC |
| | Sinai | | | | |
| CS3UTV | Cedars- | sALS | | Male | PBMC |
| | Sinai | | | | |
| CS1KL3 | Cedars- | sALS | 71 | Female | PBMC |
| | Sinai | | | | |
| CS9GXD | Cedars- | sALS | 68 | Male | PBMC |
| | Sinai | | | | |
| CS3EPR | Cedars- | sALS | 60 | Female | PBMC |
| | Sinai | | | | |
| CS4KGP | Cedars- | sALS | 82 | Female | PBMC |
| | Sinai | | | | |
| CS7MTJ | Cedars- | sALS | 66 | Female | PBMC |
| | Sinai | | | | |
| CS5ZHY | Cedars- | sALS | 64 | Male | PBMC |
| | Sinai | | | | |
| CS6EVH | Cedars- | sALS | 56 | Male | PBMC |
| | Sinai | | | | |
| CS4PFR | Cedars- | sALS | 72 | Female | PBMC |
| | Sinai | | | | |
| CS6PYD | Cedars- | sALS | 69 | Female | PBMC |
| | Sinai | | | | |

ASO Treatment of iPSC Derived Neurons

Non-targeting scrambled control (676630): CCTATAGGACTATCCAGGAA (SEQ ID NO:2), CHMP7 ASO 1 (1508916): GAAAACGGTTTCCACTGTAT (SEQ ID NO:3), CHMP7 ASO 2 (1508917): TGTTACCCTCAGATACCGCC (SEQ ID NO:4), and CHMP7 ASO 3 (1508918): ATGTGATGCTATTAATAGGA (SEQ ID NO:14) were generously provided by Ionis Pharmaceuticals. On day 25 of differentiation, ASOs were added to the culture media at concentrations of 1, 5, or 101.1M as indicated in Description of the Figures. Media was exchanged and ASO replaced every 3 days until iPSNs were subjected to downstream analyses on day 40 of differentiation for western blot, SIM, and glutamate excitotoxicity and day 46 of differentiation for stathmin-2 qRT-PCR, Ran GTPase localization, and TDP-43 localization.

### Nuclei Isolation

Nuclei were isolated from iPSNs and postmortem human brain and spinal cord tissue using the Nuclei Pure Prep Nuclei Isolation Kit (Sigma Aldrich) following manufacturer protocol as previously described (43). Briefly, iPSN lysates were prepared by rinsing iPSNs with 1×PBS, adding mL supplied lysis buffer supplemented with DTT and Triton X-100 directly to each well, and harvesting iPSNs with a cell scraper. Postmortem brain and spinal cord lysates were prepared by homogenizing 150 mg fresh frozen tissue directly in supplied lysis buffer with a dounce homogenizer. All lysates were transferred to a mL conical tube and vortexed. Sucrose gradients were assembled following manufacturer protocol. A 1.85 M sucrose gradient was used to enrich for neuronal nuclei. Samples were centrifuged at 15,600 rpm and 4° C. using a Swi32T swinging bucket rotor and Beckman ultracentrifuge (Beckman Coulter) for 45 minutes. The supernatant was discarded, and the remaining nuclei pellet was resuspended in 1 mL of supplied nuclei storage buffer to wash the nuclei of any remaining sucrose. Resuspended nuclei were centrifuged at 2500 rpm and 4° C. for 5 minutes. The supernatant was once again discarded, and the resulting nuclei pellet was vortexed in 1 mL of supplied nuclei storage buffer to resuspend for downstream imaging analysis. For western blots, washed nuclei were lysed in RIPA buffer as described below.

### Super Resolution Structured Illumination Microscopy

Nuclei staining and super resolution imaging was performed as previously described (43). Following nuclei isolation, 10-50 μL of final nuclei/storage buffer suspension was centrifuged onto collagen coated (1 mg/m; Advanced Biomatrix) slides with a CytoSpin 4 centrifuge (Thermo Fisher Scientific). Nuclei were immediately fixed with 4% PFA for 5 minutes, washed with 1×PBS 3×10 minutes and permeabilized with 1×PBST containing Triton X-100 for 15 minutes. Nuclei were then blocked with 10% normal goat serum diluted in 1×PBS for 1 hour at room temperature and incubated in primary antibody diluted in block (10% normal goat serum in 1×PBS) overnight at 4° C. (See Table H for antibody information). After 16-18 hours, nuclei were washed with 1×PBS 3×10 minutes, incubated in secondary antibody diluted in block (10% normal goat serum in 1×PBS) for 1 hour at room temperature (See Table H for antibody information). Nuclei were washed with 1×PBS 3×10 minutes and coverslipped using Prolong Gold Antifade Reagent (Invitrogen) and 18 mm×18 mm 1.5 high tolerance coverslips (MatTek).

NeuN or GFP positive nuclei (see Brief Description of the Figures) were identified via microscope eye pieces. A single z section ˜110 nm thick was acquired by widefield imaging to confirm NeuN or GFP positivity. The immunostained proteins were subsequently imaged by super resolution structured illumination microscopy (SIM) using a Zeiss ELYRA 51. For each image, 5 grid rotations and optimal z sectioning parameters were employed. For each immunostained protein, all images were acquired with identical laser power and exposure time. Following image acquisition, SIM images were reconstructed using default SIM processing parameters with Zeiss Zen Black 2.3 SP1 software. Automated nucleoporin spot and volume analysis was conducted as previously described (43, 78) using Imaris version 9.2.0 (Bitplane) and the 3D suite plugin in FIJI version 1.52p. Nucleoporin spots were counted using automated spot detection. A Bayesian classifier taking into account volume, average intensity, and contrast features was applied to detect and segment individual spots. The total number of nucleoporin spots was determined using a 3D-rendering of segmented SIM images comprising the entire depth of each nucleus. When individual nucleoporin spots could not be resolved due to limits of resolution of immunofluorescent SIM, the percent total nuclear volume occupied by the nucleoporin was calculated. To calculate total nuclear volume, X and Y axis length was measured in the center z-slice for each nucleus and Z axis length was estimated from the total z depth of acquired images. To calculate nucleoporin volume, image stacks were processed with automatic thresholding and automatic thresholding the 3D suite plugin in FIJI was used to determine the volume of the thresholded area for each nucleus. Representative images are presented as 3D maximum intensity projections generated in Zeiss Zen Black 2.3 SP1. Images were faux colored green for contrast and display.

TABLE H
Antibody Information
Primary Antibodies

| | | Catalog | Application and |
|---|---|---|---|
| Antibody | Source | Number | Concentration |
| Mouse Anti-CHMP7 | Santa Cruz | sc-271805 | IF: 1/50 |
| | Biotechnology | | Western: 1/250 |
| Rabbit Anti-CHMP7 | Thermo Fisher | PA557525 | IF: 1/250 |
| | Scientific | | |
| Rabbit Anti-CHMP7 | Sigma Aldrich | HPA036119 | IF: 1/250 |
| | | | Western: 1/1000 |
| Rabbit Anti-Nup50 | Abcam | ab137092 | IF: 1/250 |
| Rabbit Anti-Nup153 | Abcam | ab84872 | IF: 1/200 |
| Mouse Anti-TPR | Santa Cruz | sc-271565 | IF: 1/50 |
| | Biotechnology | | |
| Rat Anti-Nup62 | Millipore | MABE1043 | IF: 1/250 |
| Rabbit Anti-POM121 | Novus | NBP2-19890 | IF: 1/250 |
| | Biologicals | | |
| Mouse Anti-Nup133 | Santa Cruz | Sc-376699 | IF: 1/50 |
| | Biotechnology | | |
| Mouse Anti-414 (FG | Abcam | Ab50008 | IF: 1/250 |
| Nups) | | | |
| Rabbit Anti-LEMD2 | Thermo Fisher | PA553589 | IF: 1/250 |
| | Scientific | | |
| Rabbit Anti-LEMD2 | Sigma Aldrich | HPA017340 | IF: 1/250 |
| | | | Western: 1/1000 |
| Rabbit Anti-TDP-43 | ProteinTech | 10782-2-AP | IF: 1/250 |
| Mouse Anti-TDP-43 | Abcam | ab104223 | IF: 1/250 |
| Mouse Anti-Ran | BD Biosciences | 610341 | IF: 1/200 |
| Chicken Anti-NeuN | Millipore | ABN91 | IF: 1/500 |
| Guinea Pig Anti-Map2 | Synaptic | 188004 | IF: 1/1000 |
| | Systems | | |
| Chicken Anti-GFP | Millipore | AB16901 | IF: 1/1000 |
| Mouse Anti-Turbo GFP | Origene | TA150041 | IF: 1/250 |
| Rabbit Anti-Turbo GFP | Thermo Fisher | PA5-22688 | IF: 1/250 |
| | Scientific | | |
| Rabbit Anti-Lamin B1 | Abcam | ab16048 | Western: 1/1000 |
| Mouse Anti-GAPDH | Life | AM4300 | Western: |
| | Technologies | | 1/10,000 |

Secondary Antibodies

| | | | |
|---|---|---|---|
| Goat Anti-Mouse Alexa | Invitrogen | A11029 | IF: 1/1000 |
| 488 | | | |
| Goat Anti-Rabbit Alexa | Invitrogen | A11034 | IF: 1/1000 |
| 488 | | | |
| Goat Anti-Rat Alexa 488 | Invitrogen | A11006 | IF: 1/1000 |
| Goat Anti-Guinea Pig | Invitrogen | A11073 | IF: 1/1000 |
| Alexa 488 | | | |
| Goat Anti-Chicken | Invitrogen | A11039 | IF: 1/1000 |
| Alexa 488 | | | |
| Goat Anti-Mouse Alexa | Invitrogen | A11031 | IF: 1/1000 |
| 568 | | | |
| Goat Anti-Rabbit Alexa | Invitrogen | A11036 | IF: 1/1000 |
| 568 | | | |
| Goat Anti-Rat Alexa 568 | Invitrogen | A11077 | IF: 1/1000 |
| Goat Anti-Guinea Pig | Invitrogen | A11075 | IF: 1/1000 |
| Alexa 568 | | | |
| Goat Anti-Mouse Alexa | Invitrogen | A21236 | IF: 1/1000 |
| 647 | | | |
| Goat Anti-Rabbit Alexa | Invitrogen | A21245 | IF: 1/1000 |
| 647 | | | |
| Goat Anti-Guinea Pig | Invitrogen | A21450 | IF: 1/1000 |
| Alexa 647 | | | |
| Goat Anti-Chicken | Invitrogen | A21449 | IF: 1/1000 |
| Alexa 647 | | | |
| Donkey Anti-Rabbit IgG | Thermo Fisher | 45-000-682 | Western: 1/5000 |
| HRP | Scientific | | |
| Horse Anti-Mouse IgG | Cell Signaling | 7076S | Western: 1/5000 |
| HRP | | | |

Immunostaining and Confocal Imaging of iPSNs

On day 12 of differentiation, iPSNs were plated in 24 well optical bottom plates (Cellvis). At day 32 (CHMP7 OE) or 46 of differentiation (CHMP7 ASO), iPSNs were fixed in 4% PFA for 15 minutes, washed with 1×PBS 3×10 minutes, permeabilized with 1×PBST containing 0.3% Triton X-100 for 15 minutes, blocked in 10% normal goat serum diluted in 1×PBS for 1 hour, and incubated in primary antibody for 2 hours at room temperature (See Table H for antibody information). iPSNs were then washed with 1×PBS 3×10 minutes, incubated in secondary antibody (See Table H for antibody information) for 1 hour at room temperature, washed in 1×PBS 2×10 minutes, incubated with Hoescht diluted 1:1000 in 1×PBS for 10 minutes, and washed in 1×PBS 2×10 minutes. iPSNs were mounted using Prolong Gold Antifade Reagent with DAPI. iPSNs were imaged using a Zeiss LSM 800 or Zeiss LSM 980 confocal microscope. All images were acquired using identical imaging parameters (e.g. laser power, gain). Nuclear intensity and N/C ratios were calculated with FIJI as previously described (80). Images presented are maximum intensity projections generated in Zeiss Zen Blue 2.3.

### Human Tissue Immunofluorescence

Non-neurological control and C9orf72 patient postmortem paraffin embedded motor and occipital cortex sections were obtained through the Target ALS Human Postmortem Tissue Core (see Table I for demographic information). Tissue sections were gradually rehydrated with xylene 3×5 minutes, 100% ethanol 2×5 minutes, 90% ethanol 5 minutes, 70% ethanol 5 minutes, and finally dH₂O 3×5 minutes. Antigen retrieval was performed with Tissue-Tek antigen retrieval solution (IHC World) for 1 hour in a steamer. Slides were cooled for 10 minutes and washed 3×5 minutes with dH₂O, 2×5 minutes 1×PBS, and permeabilized with 0.4% Triton X-100 diluted in 1×PBS for 10 minutes on a shaker. Slides were subsequently washed 3×5 minutes in 1×PBS blocked with DAKO protein-free serum block (DAKO) overnight at 4° C. Tissue sections were incubated with primary antibody (see Table H for antibody information) diluted in DAKO antibody diluent reagent with background reducing components for a total of 2 overnights at 4° C. In between the 2 overnight incubations, slides were incubated in primary antibody at room temperature with gentle agitation for 10 hours. Following primary antibody, tissue sections were washed 3×5 minutes with 1×PBS and then incubated with secondary antibody (see Table H for antibody information) diluted in DAKO antibody diluent with background reducing components (DAKO) at room temperature with gentle agitation for 1 hour. Slides were then washed 3×5 minutes in 1×PBS, rinsed briefly with 2-3 drops autofluorescence eliminator reagent (Millipore), and extensively washed 5×5 minutes in 1×PBS to remove debris. Tissue sections were stained with Hoescht diluted 1:1000 in 1×PBS for 20 minutes, and washed 3×5 mins in 1×PBS. Slides were cover slipped using Prolong Gold Antifade Reagent with DAPI and nuclei from Map2 positive Layer V neurons were imaged with a 20× objective and a Zeiss Axioimager Z2 fluorescent microscope equipped with an apotome2 module. All images were acquired using identical exposure times.

CHMP7 and TDP-43 nuclear/cytoplasmic ratios were quantified using FIJI. Briefly, in focus neurons with visible nuclei were identified by positive Map2 and Hoescht signals. A small pixel box was used to measure fluorescent intensity in the CHMP7 channel. Two integrated density measurements were taken from each cellular compartment (nucleus and cytoplasm), the average of which was used for subsequent analysis. Five background fluorescent intensity mean measurements were taken in areas devoid of cells, the average of which was used for analysis. Nuclear and cytoplasmic intensities were calculated as follows: Intensity=Integrated Density−(Mean Background Intensity*Area Measured Within Cell). The nuclear/cytoplasmic CHMP7 ratio was calculated by dividing resulting nuclear intensity by cytoplasmic intensity for each cell. Images are presented as default apotome processed images generated in Zeiss Zen Blue 2.3.

TABLE I
Demographic Information for Postmortem Human Tissue

| | Clinical Diagnosis | Age of Death | Sex |
|---|---|---|---|
| Control-1 | Non-neurologic control | | Female |
| Control-2 | Non-neurologic control | 70 | Female |
| Control-3 | Non-neurologic control | 70 | Male |
| Control-4 | Non-neurologic control | 59 | Male |
| Control-5 | Non-neurologic control | 71 | Female |
| Control-6 | Non-neurologic control | 52 | Male |
| Control-7 | Non-neurologic control | 72 | Male |
| Control-8 | Non-neurologic control | 74 | Female |
| Control-9 | Non-neurologic control | 92 | Female |
| Control-10 | Non-neurologic control | 37 | Female |
| Control-11 | Non-neurologic control | 50 | Male |
| Control-12 | Non-neurologic control | 54 | Female |
| Control-13 | Non-neurologic control | 49 | Male |
| C9orf72-1 | C9orf72 | 72 | Male |
| C9orf72-2 | C9orf72 | 69 | Female |
| C9orf72-3 | C9orf72 | 61 | Female |
| C9orf72-4 | C9orf72 | 51 | Female |
| C9orf72-5 | C9orf72 | 59 | Male |
| C9orf72-6 | C9orf72 | 61 | Female |
| C9orf72-7 | C9orf72 | 68 | Female |
| C9orf72-8 | C9orf72 | 58 | Female |
| C9orf72-9 | C9orf72 | 77 | Female |
| C9orf72-10 | C9orf72 | 63 | Male |
| C9orf72-11 | C9orf72 | 63 | Male |
| C9orf72-12 | C9orf72 | 59 | Male |
| C9orf72-13 | C9orf72 | 62 | Female |
| C9orf72-14 | C9orf72 | 58 | Male |
| C9orf72-15 | C9orf72 | 70 | Female |
| C9orf72-16 | C9orf72 | 67 | Male |
| C9orf72-17 | C9orf72 | 67 | Female |
| sALS-1 | sALS | 69 | Male |
| sALS-2 | sALS | 68 | Female |
| sALS-3 | sALS | 69 | Male |
| sALS-4 | sALS | 67 | Female |
| sALS-5 | sALS | 59 | Female |
| sALS-6 | sALS | 68 | Male |
| sALS-7 | sALS | 71 | Female |
| sALS-8 | sALS | 66 | Male |
| sALS-9 | sALS | 70 | Female |
| sALS-10 | sALS | 82 | Male |
| sALS-11 | sALS | 48 | Male |
| sALS-12 | sALS | 58 | Female |
| sALS-13 | sALS | 60 | Female |
| sALS-14 | sALS | 72 | Female |
| sALS-15 | sALS | 56 | Female |
| sALS-16 | sALS | 52 | Male |
| sALS-17 | sALS | 50 | Male |
| sALS-18 | sALS | 62 | Female |
| sALS-19 | sALS | 70 | Male |
| sALS-20 | sALS | 62 | Male |
| sALS-21 | sALS | 64 | Female |
| sALS-22 | sALS | 69 | Female |
| sALS-23 | sALS | 60 | Male |
| sALS-24 | sALS | 73 | Female |
| sALS-25 | sALS | 66 | Male |
| sALS-26 | sALS | 71 | Female |
| sALS-27 | sALS | 79 | Male |
| sALS-28 | sALS | 77 | Female |
| sALS-29 | sALS | 70 | Male |
| sALS-30 | sALS | 63 | Male |

qRT-PCR

RNA from iPSNs was isolated with a RNeasy kit (QIAGEN) according to manufacturer protocol. RNA concentrations were determined with a NanoDrop 1000 spectrophotometer (Thermo Fisher Scientific). For CHMP7 ASO experiments, RNA was isolated on day 46 of differentiation following 3 weeks exposure to 5 μM CHMP7 ASO2 or scrambled control ASO. For CHMP7 overexpression experiments, RNA was isolated on day 32 of differentiation (2 weeks after plasmid nucleofection). 1 μg of RNA was used for cDNA synthesis with random hexamers and the Superscript IV First-Strand cDNA Synthesis Kit (Thermo Fisher Scientific). qRT-PCR reactions were conducted as previously described (70, 71) using Sybr Green Master Mix (Thermo Fisher Scientific), an Applied Biosystems Step One Plus Real Time PCR Machine (Thermo Fisher Scientific). See Table J for primer sequences. GAPDH was used for normalization in all experiments.

TABLE J
Primer Sequences for qRT-PCR

| Target | Sequence | |
|---|---|---|
| Full length | Forward | AGCTGTCCATGCTGTCACTG (SEQ ID NO: 15) |
| STMN2 | Reverse | GGTGGCTTCAAGATCAGCTC (SEQ ID NO: 16) |
| Truncated | Forward | GGACTCGGCAGAAGACCTTC (SEQ ID NO: 17) |
| STMN2 | Reverse | GCAGGCTGTCTGTCTCTCTC (SEQ ID NO: 18) |
| GAPDH | Forward | GAAGGTGAAGGTCGGAGTC (SEQ ID NO: 19) |
| | Reverse | GAAGATGGTGATGGGATTTC (SEQ ID NO: 20) |

### Western Blots

Nuclei Lysates: Following nuclei isolation, nuclei pellets were resuspended in 25 μL RIPA buffer (Millipore) containing 1× protease inhibitor cocktail (Roche). Homogenates were spun at 12,000 g for 15 minutes and 4° C. to remove debris. The supernatant was transferred to a new Eppendorf tube and protein concentrations were determined using the BCA protein estimation assay kit (Thermo Fisher Scientific). 4× Laemmli buffer (BioRad) was added to each sample to a final concentration of 1×, samples were heated at 100° C. for 5 minutes, and 5 μg protein was loaded into 4-20% acrylamide gels (BioRad). Gels were run until the dye front reached the bottom. Protein was transferred onto a nitrocellulose membrane using the Trans-Blot Turbo Transfer System (BioRad). Blots were blocked for 30 minutes with 5% non-fat milk in 1×TBST (0.1% Tween-20) and incubated overnight at 4° C. with primary antibody diluted in block (see Table H for antibody information). The next day, blots were washed 4×10 minutes with 1×TBST and probed with secondary antibody diluted in block (see Table H for antibody information) for 1 hour at room temperature. Blots were subsequently washed 4×10 minutes with 1×TBST and ECL substrate (Thermo Fisher Scientific, Millipore) was applied for 30 seconds. Chemiluminescent images were acquired with the GE Healthcare ImageQuant LAS 4000 system. To sequentially probe membranes without stripping, chemiluminescent signals were quenched by incubating blots in room temperature 30% H₂O₂ for 15 minutes (90). Analysis was conducted in FIJI. Lamin B1 was used for normalization.

ASO Treated iPSN Lysates: On day 40 of differentiation, iPSNs were rinsed with ice cold 1×DPBS with Ca²⁺ and Mg²⁺. 1 mL fresh 1×DPBS was added to each well and iPSNs were harvested with a cell scraper and transferred to an Eppendorf tube. Cells were then pelleted at 2500 rpm, 4° C. for 5 minutes. The supernatant was aspirated, and the resulting cell pellet was lysed in 50 μL RIPA buffer containing 1× protease inhibitor cocktail. Western blots and analysis were performed as described above. GAPDH was used for normalization.

### Plasmids and Nucleofection for CHMP7 Overexpression

The GFP tagged CHMP7 plasmid was obtained from Origene. To generate the CHMP7 NES mutant (CHMP7 NES*) plasmid, a single amino acid substitution (amino acid 430, L to A) was created in the CHMP7 GFP plasmid (Origene) using the Q5 site directed mutagenesis kit (New England BioLabs). A GFP control plasmid was obtained from Origene. See Table K for plasmid information. On day 18 of differentiation, iPSNs were dissociated with accutase to assist with single cell dissociation and nucleofected in suspension using the Lonza P3 Primary Cell 4D Nucleofector Kit (Lonza) and program DC104 on the Lonza nucleofection system. Each cuvette contained 5×10⁶ iPSNs and 4 μg plasmid DNA. Following nucleofection, iPSNs were plated in Matrigel (Corning) coated cell culture dishes according to Lonza protocol. Media was exchanged on day 19 and 22 of differentiation. For overexpression of CHMP7 variants, downstream SIM experimentation was carried out on day 25 of differentiation and TDP-43 localization and stathmin-2 analyses were carried out on day 32 of differentiation. For overexpression of LEMD2, SIM experiments were carried out on day 32 of differentiation.

TABLE K
Plasmid Information

| Plasmid | Backbone | Catalog Number |
|---|---|---|
| CHMP7 GFP | pCMV6-AC-GFP | Origene RG222461 |
| CHMP7 GFP | pCMV | Addgene 97006 |
| CHMP7 NES* GFP | pCMV6-AC-GFP | N/A, this study |
| GFP | pCMV6-AC-GFP | Origene PS100010 |
| Trim21 GFP | pCMV6-AC-GFP | Origene RG202088 |
| LEMD2 GFP | pCMV6-AC-GFP | Origene RG208535 |

### Knockdown of POM121 and LEMD2 by Trim Away

Knockdown of endogenous POM121 and LEMD2 was carried out using a modified Trim Away protocol (58) as previously described (43). POM121 (Thermo Fisher Scientific) and LEMD2 (Thermo Fisher Scientific) antibodies were dialyzed in 1×PBS using a Slide-A-Lyzer MINI Dialysis Device, 20K MWCO (Thermo Fisher Scientific). PBS was exchanged after 2 hours and dialysis proceeded with gentle agitation at room temperature overnight. Dialyzed antibodies were concentrated with an Amicon Ulta-0.5 Centrifugal Filter Unit with Ultacell-100 membrane (Millipore). Resulting antibody concentration was calculated with a NanoDrop 1000 spectrophotometer (Thermo Fisher Scientific) as previously described (58). On day 18 of differentiation, iPSNs were dissociated with accutase. Suspension based nucleofection was carried out with the Lonza P3 Primary Cell 4D Nucleofector Kit (Lonza) and the program DC154. Each cuvette contained 5×10 6 iPSNs, 5 lag of antibody and 4 μg Trim21 GFP plasmid DNA (Origene, see Table K). Following nucleofection, iPSNs were replated in Matrigel (Corning) coated cell culture dishes according to Lonza protocol and used for downstream analysis on day 20 of differentiation.

### Glutamate Toxicity

On day 12 of differentiation, iPSNs were plated in 24 well optical bottom plates (Cellvis) at a density of 250,000 neurons per well. Neurons were rinsed with 1×PBS and fed with fresh stage 3 media daily remove dead cells and debris until day 25 of differentiation. On day 25 of differentiation, ASO treatment was initiated as described above. Every 3 days, iPSNs were rinsed 3× with 1×PBS and media and ASO were replaced. On day 40 of differentiation, iPSNs were washed with 1×PBS to remove any remaining debris and dead cells. Media was replaced with ACSF (Tocris) containing 0 or 10 μM glutamate (Sigma Aldrich). iPSNs were incubated at 37° C. with 5% CO₂ for 4 hours. After 3.5 hours, one drop of NucBlue Live ReadyProbes (Thermo Fisher Scientific) and 1 μM propidium iodide (Thermo Fisher Scientific) and returned to the incubator for 30 minutes. iPSNs were imaged in an environmentally controlled chamber with a Zeiss LSM 800 confocal microscope. 5 images per well were acquired with a 10× objective. PI and DAPI spots were counted using FIJI.

### Statistical Analysis

All data analysis was conducted with Imaris or FIJI as described in each experimental section above and was either completely automated or blinded. All statistical analyses were performed using GraphPad Prism version 8 (GraphPad). For imaging experiments where multiple cells or nuclei per iPSC line or patient were quantified, statistical analyses were performed such that the average of all nuclei or cells evaluated per iPSC line or patient represents N=1 with total N per experiment and group as indicated in Description of the Figures. Student's t-test, One-way ANOVA with Tukey's multiple comparison test, or Two-way ANOVA with Tukey's multiple comparison test was used as described in Description of the Figures. * p<0.05, ** p<0.005, *** p<0.0005, **** p<0.0001. Violin plots are used to display the full spread and variability of large data sets (>10 data points). Center dotted line indicates median value. Two additional dotted lines indicate the 25^{th} and 75^{th} percentiles. For smaller data sets, bar graphs displaying individual data points shown where error bars represent +/−SEM.

### Example 9—Specific Nucleoporins are Reduced in sALS iPSN Nuclei

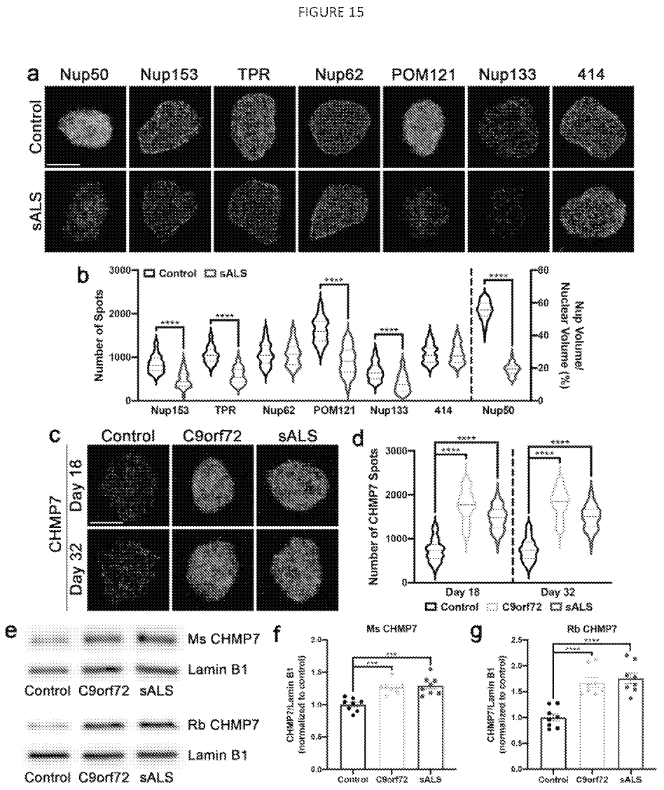

Recently, it was established that the protein expression of eight specific Nups is reduced within the nucleoplasm and NPCs of C9orf72 human neurons (43). Given that multiple pathologies and molecular pathways are commonly linked to both familial and sporadic disease (36, 38, 55), it was hypothesized that Nup reduction may also be a prominent pathological feature in sporadic disease. Therefore, we selected a subset of candidate Nups spanning multiple sub-domains of the NPC based on our previous studies in C9orf72 disease and visually examined their nuclear distribution and expression using super resolution structured illumination microscopy (SIM) in nuclei isolated from sALS iPSNs. SIM is capable of providing an estimated resolution of 100 nm, approximating the dimensions of an individual human NPC (45, 56, 57), thus providing us the unparalled opportunity to reliably evaluate the ascertain the NPC localization and expression of candidate Nups via conventional immunostaining procedures. Using this methodology, we found that a subset of Nups are reduced from the nucleoplasm and NPCs of NeuN positive sALS iPSN nuclei (FIG. 15A-15B). In contrast, we observed no overt changes to FG-Nup distribution or expression as evaluated by immunostaining with an antibody recognizing multiple FG-containing Nups, mAb414 (FIGS. 15A-15B). Notably, the pathological reduction in Nup50, TPR, POM121, and Nup133 in sALS neuronal nuclei (FIGS. 15A-15B) was highly reminiscent of our observations in C9orf72 disease (43) suggesting that common molecular events may underlie Nup reduction in familial and sporadic disease.

### Example 10—the Nuclear Localization and Expression of CHMP7 is Increased in C9orf72 and sALS iPSNs and Postmortem Human Motor Cortex

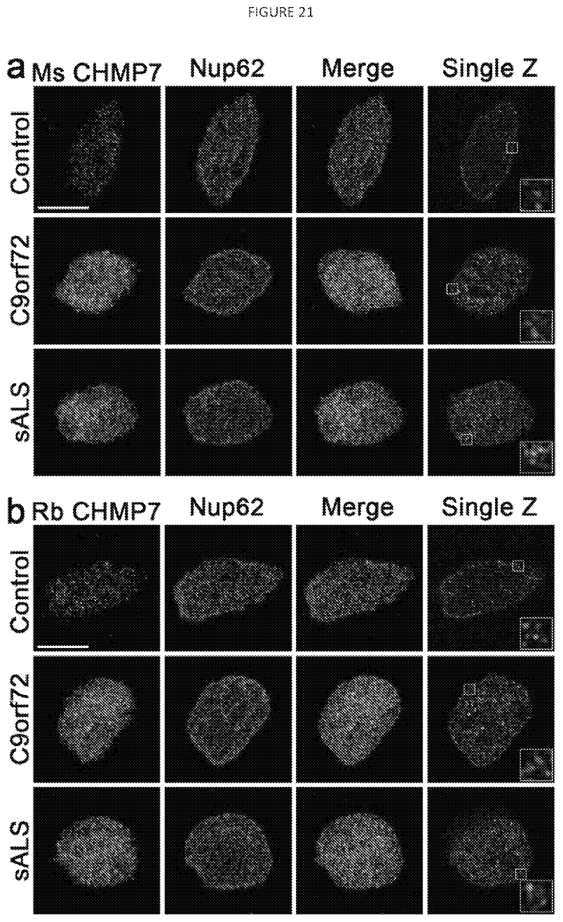

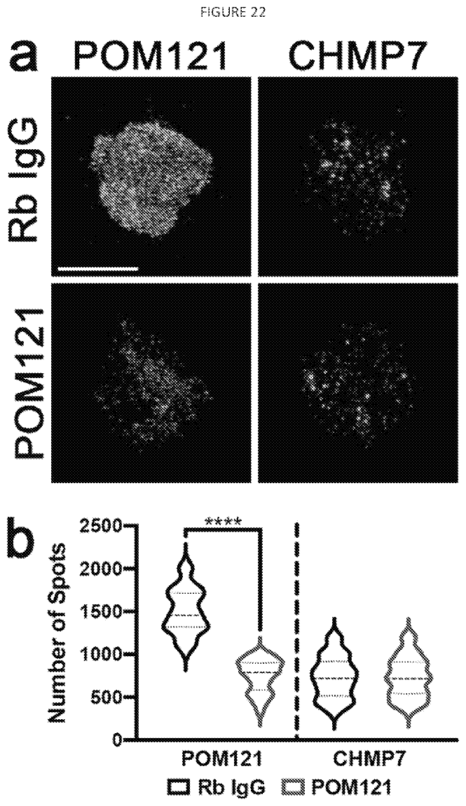

Given the critical involvement of CHMP7 in NPC homeostasis in yeast (48-50), we investigated whether the expression of this protein was pathologically altered in C9orf72 and sALS nuclei. Using SIM, we found a significant increase in CHMP7 immunoreactivity in NeuN positive nuclei isolated from control and C9orf72 and sALS iPSNs (FIGS. 15C-15D). While CHMP7 does rarely localize in close proximity to Nup62 positive NPCs and spots, its increased expression, as evaluated by immunostaining with two independent antibodies, appears predominantly nucleoplasmic in C9orf72 and sALS iPSNs (FIG. 21). Additionally, western blot analyses using two anti-CHMP7 antibodies quantitatively confirmed an increase in CHMP7 protein levels in C9orf72 and sALS nuclei (FIGS. 15E-G). Interestingly, this increase was observed even at a time point (Day 18 of differentiation, FIGS. 15C-D) preceding that at which Nup reduction was previously observed (FIGS. 15A-B); (43). We have previously demonstrated that disease associated NPC injury can be initiated by rapid Trim21 GFP mediated degradation (58) of the transmembrane Nup POM121 (43). Here, we found that rapid reduction of POM121 from wildtype iPSN nuclei did not result in increased nuclear CHMP7 expression (FIG. 22). Together, these data suggest that CHMP7 is not responding to NPC injury but may play a more direct role in its initiation.

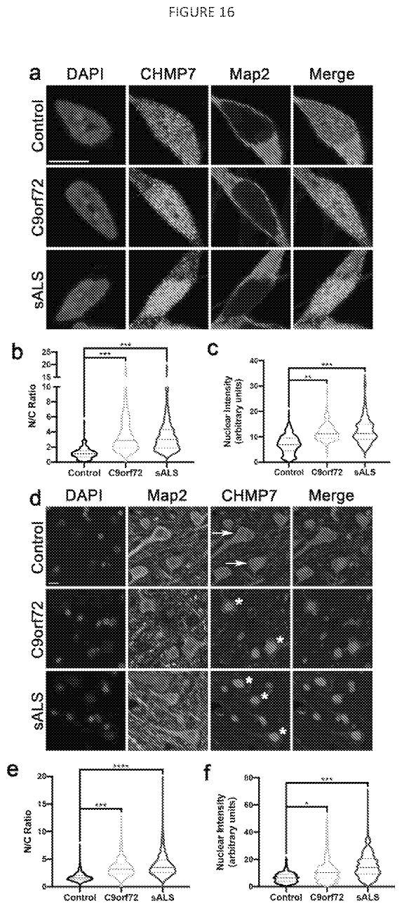

Given that the nuclear/cytoplasmic partitioning of multiple proteins is disrupted in multiple neurodegenerative diseases (59-62), we next evaluated the nuclear and cytoplasmic distribution of CHMP7 in our disease iPSNs. Using immunostaining and confocal imaging, we observed a significant relocalization of CHMP7 from the cytoplasm to the nucleus in C9orf72 and sALS iPSNs (FIGS. 16A-16B). Consequently, similar to our observations in isolated nuclei (FIGS. 15C-15D), this corresponded to an increase in overall CHMP7 nuclear intensity in C9orf72 and sALS iPSNs compared to controls (FIGS. 16A-16C). To verify that our results in iPSNs recapitulated human disease pathology, we performed immunofluorescent staining in thin paraffin embedded sections. This revealed a significant relocalization of CHMP7 to the nucleus (FIGS. 16D-16E) and overall increase in CHMP7 nuclear intensity (FIGS. 16D, 16F) in Map2 positive neurons in postmortem patient motor cortex mimicking our data obtained in iPSNs.

### Example 11—Manipulation of LEMD2 Levels does not Impact the Nuclear Localization or Expression of CHMP7 in iPSNs

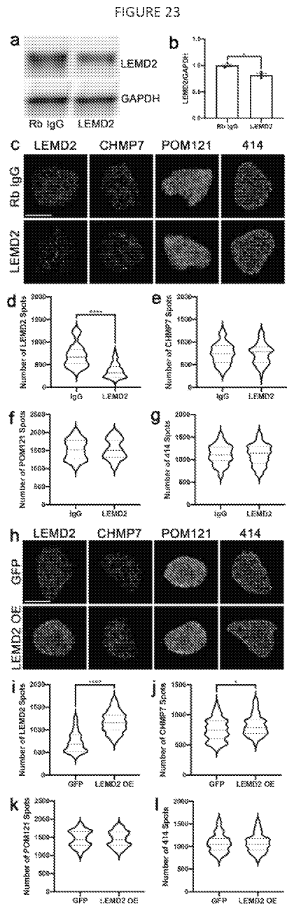

Having established that increased nuclear CHMP7 is a robust and prominent molecular feature of sporadic ALS and familial ALS/FTD, we next sought to determine the mechanism by which this pathology occurred in human neuronal nuclei. As both the yeast and human CHMP7 orthologues are recruited to the nuclear envelope by the integral INM protein LEMD2 (50, 63) in non-neuronal cell types, we genetically manipulated LEMD2 levels in control iPSNs using the Trim Away method to reduce endogenous LEMD2 levels or GFP tagged expression plasmids to overexpress LEMD2. We found that neither a small but significant reduction in nuclear LEMD2 (FIGS. 23A-23B) nor overexpression of GFP tagged LEMD2 has an effect on the nuclear immunoreactivity of CHMP7 or specific Nups using SIM (FIGS. 23C-23L). Collectively, this data suggests that LEMD2 mediated recruitment of CHMP7 to the nuclear envelope is not responsible for increased nuclear expression of CHMP7 in disease iPSNs. Additionally, the nuclear recruitment of CHMP7 may be regulated via distinct molecular mechanisms in human neurons compared to yeast and non-neuronal mammalian cells.

### Example 12—Inhibition of CHMP7 Nuclear Export Recapitulates Disease Associated Reduction in Specific Nucleoporins and Induces TDP-43 Mislocalization and Altered Stathmin-2 Splicing

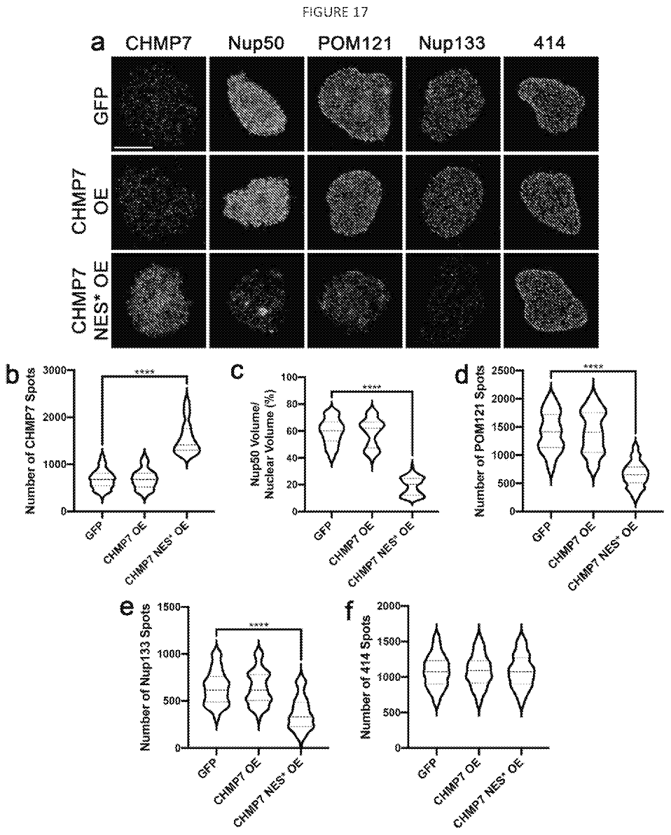

In unperturbed conditions, both yeast and human CHMP7 cannot accumulate in the nucleus due to its nuclear export by XPO1/CRM1 (49, 64). Therefore, to determine whether specifically inhibiting CHMP7 nuclear export could initiate a reduction in Nup levels in iPSNs, we mutated the nuclear export sequence (NES) of CHMP7 by making a single amino acid substitution (L to A, amino acid 430) in the CHMP7 NES sequence (LEAELEKLSLS). As previously described (49), this single point mutation is sufficient to abrogate XPO1 mediated nuclear export of CHMP7 (65). Overexpression of the GFP tagged CHMP7 NES mutant (CHMP7 NES*), but not wildtype GFP tagged CHMP7, in wildtype iPSNs recapitulated C9orf72 and sALS disease associated alterations in nuclear CHMP7, Nup50, POM121, and Nup133 levels as evaluated by SIM (FIG. 17). Consistent with a role for as an early-acting factor that mediates Nup and NPC degradation (48-50), these data suggest that aberrant nuclear accumulation of CHMP7 is sufficient to facilitate alterations to Nup levels in iPSN nuclei.

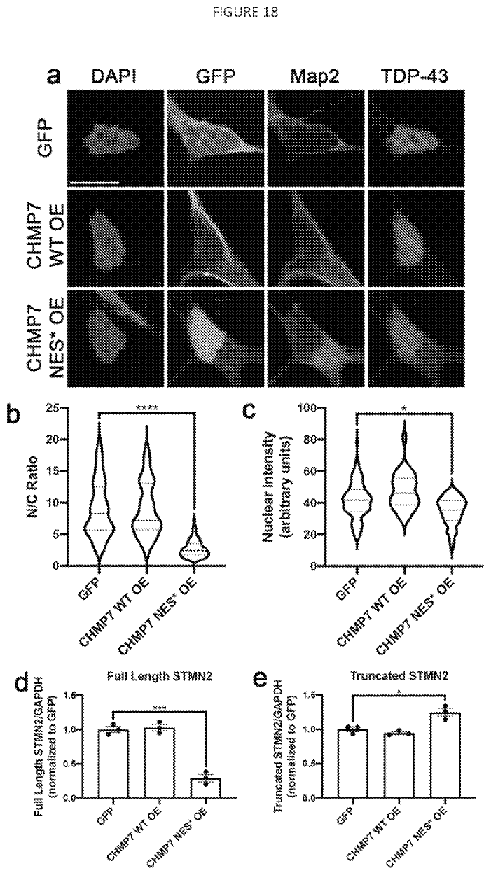

TDP-43 is a DNA and RNA binding protein that while normally predominantly nuclear, it is observed to be cleared from the nucleus and/or mislocalized (and in some cases subsequently accumulated) in the cytoplasm within a subset of neurons of ˜97% of ALS. It is thought that the nuclear clearance and cytoplasmic mislocalization leads to a loss of TDP-43 function as a pathogeneic event in multiple neurodegenerative diseases including sporadic ALS (36, 66). Through associations with nuclear transport receptors known as karyopherins, the NPC and its individual Nup components are known to critically govern the nucleocytoplasmic transport (NCT) of protein and RNA species into and out of the nucleus (45, 46, 65). Given that TDP-43 shuttles between the nucleus and cytoplasm to regulate the metabolism of its mRNA targets (68, 69), we hypothesized that perhaps NPC injury may at least in part contribute to TDP-43 mislocalization and dysfunction in human neurons. To test this, we first overexpressed GFP tagged CHMP7 variants in wildtype iPSNs and monitored the localization of endogenous TDP-43 via immunostaining and confocal microscopy. Overexpression of GFP tagged wildtype CHMP7 had no effect on the subcellular localization of endogenous TDP-43 (FIGS. 18A-18-C). In contrast, overexpression of our GFP tagged CHMP7 NES mutant (CHMP7 NES*) resulted in a significant relocalization of TDP-43 from the nucleus to the cytoplasm in control iPSNs as indicated by a decreased TDP-43 nuclear/cytoplasmic ratio and decreased nuclear intensity of TDP-43 immunostaining (FIG. 18A-18C). Recently, alterations in stathmin-2 mRNA splicing have been identified as a prominent pathological consequence of loss of TDP-43 function (70, 71). To evaluate whether TDP-43 was disrupted upon overexpression of our GFP tagged CHMP7 NES* construct, qRT-PCR was performed for full length and truncated stathmin-2 mRNA species. While overexpression of wildtype CHMP7 had no impact on stathmin-2 mRNA splicing, CHMP7 NES* overexpression resulted in a significant decrease in full length stathmin-2 mRNA and a corresponding increase in truncated stathmin-2 mRNA (FIGS. 18D-18E) consistent with a loss of TDP-43 function (70, 71). Together, these data indicate that nuclear retention of CHMP7 is sufficient to initiate a pathological cascade impacting nuclear Nup expression, and TDP-43 localization and function in human neurons.

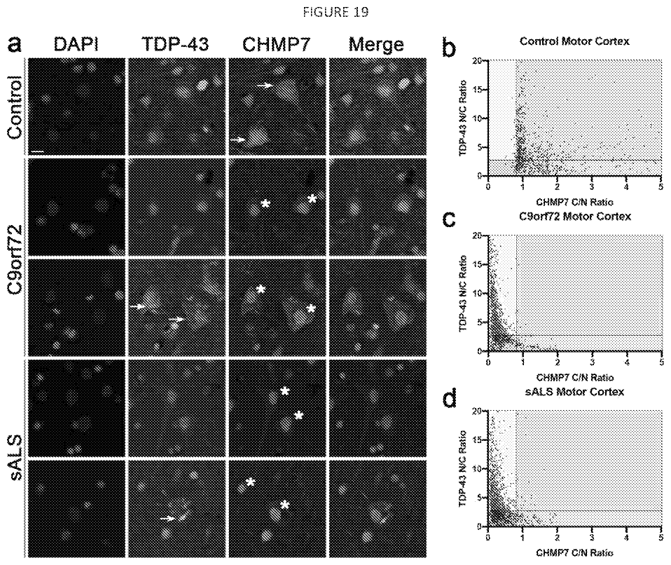

Next, to investigate whether CHMP7 and TDP-43 co-pathology could be observed in real human disease, double immunostaining in thin paraffin embedded sections from postmortem patient motor cortex was performed (FIG. 19A). Following immunostaining and imaging, the nuclear/cytoplasmic ratio of TDP-43 immunostaining was quantitatively compared to the cytoplasmic/nuclear ratio of CHMP7 immunostaining in individual Map2 positive neurons (FIGS. 19B-19B). Consistent with reports that TDP-43 mislocalization can be observed in normal aging (72) or as a result of non-degenerative neuronal injury (73, 74), a subset of neurons in non-neurologic control motor cortex tissue displayed a decreased nuclear/cytoplasmic TDP-43 immunoreactivity independent of CHMP7 pathology (FIG. 19B, blue box). In contrast, in disease motor cortex, it was found that about 80-90% of Map2 positive neurons in C9orf72 and sALS patient motor cortex display increased nuclear CHMP7 immunostaining (FIG. 19). A subset of these CHMP7 pathology positive neurons, also displayed a decreased nuclear/cytoplasmic ratio of TDP-43 immunostaining (FIGS. 19A, 19C-19D). Notably, cytoplasmic TDP-43 aggregation was observed in a handful of neurons with increased nuclear/cytoplasmic CHMP7 immunoreactivity (FIG. 19A). Intriguingly, a large portion of the neurons evaluated were positive for CHMP7 pathology but negative for TDP-43 pathology (FIG. 19) consistent with our hypothesis that increased nuclear CHMP7 is an initiating event in neuronal injury in neurodegeneration.

### Example 13—ASO Mediated Knockdown of CHMP7 Mitigates Nup Alterations, Alleviates Deficits in Stathmin-2 Splicing, and Improves Neuronal Survival

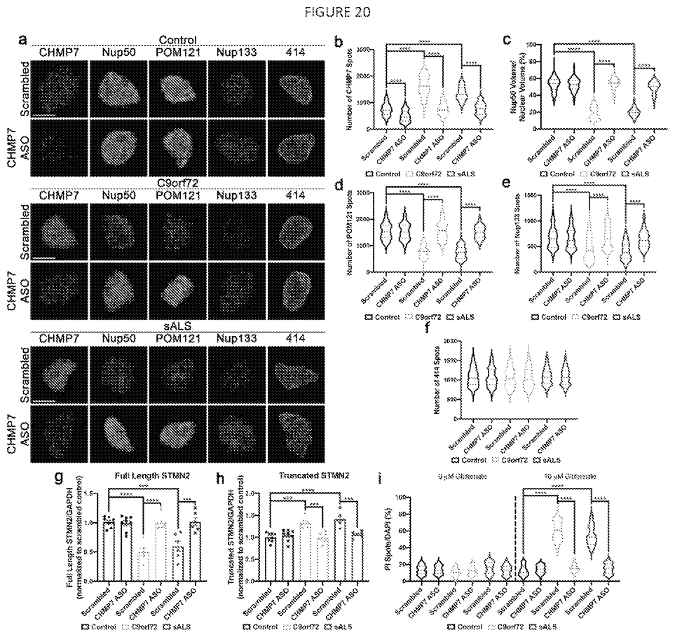

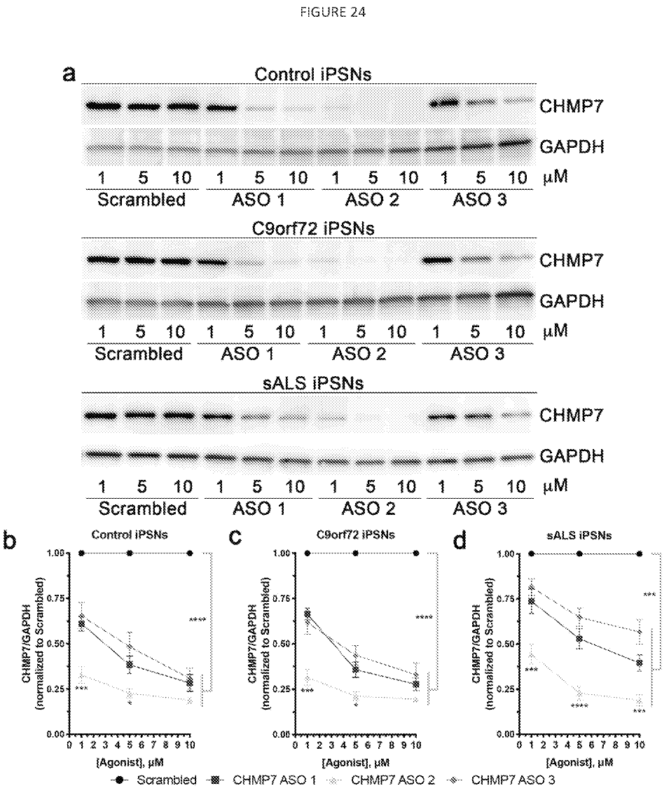

Our data suggest that therapeutically reducing CHMP7 could prove beneficial for neurodegenerative disease characterized by Nup alterations and TDP-43 dysfunction. ASOs are now commonly used as a therapeutic strategy to reduce mRNA and protein levels of genes of interests (75, 76). To evaluate if this therapeutic strategy would effectively mitigate NPC injury, ASOs that specifically target human CHMP7 pre-mRNA and induce its degradation via RNase H based mechanisms were designed. Compared to scrambled control ASOs, 2-week exposure to 3 different CHMP7 targeting ASOs, significantly reduced CHMP7 protein levels in control and C9orf72 iPSNs in a dose dependent manner (FIG. 24). Consistent with our western blot experiments using whole iPSN lysates (FIG. 24), SIM analyses revealed that CHMP7 ASO treatment significantly reduced nuclear levels of CHMP7 in control, C9orf72, and sALS iPSNs (FIGS. 20A-20B). Moreover, compared to a scrambled control ASO, 5 μM CHMP7 ASO 2 significantly restored POM121, Nup133, and Nup50 expression in C9orf72 and sALS iPSN nuclei and NPCs (FIGS. 20A, 20C-20F) as evaluated by SIM. Importantly, no disruption was observed in Nups in control iPSNs treated with CHMP7 ASOs (FIGS. 20A, 20C-20F) nor on FG Nups in C9orf72 or sALS iPSNs (FIGS. 20A,20F) suggesting that ASO mediated knockdown in CHMP7 protein levels specifically mitigates disease associated Nup alterations in iPSNs.

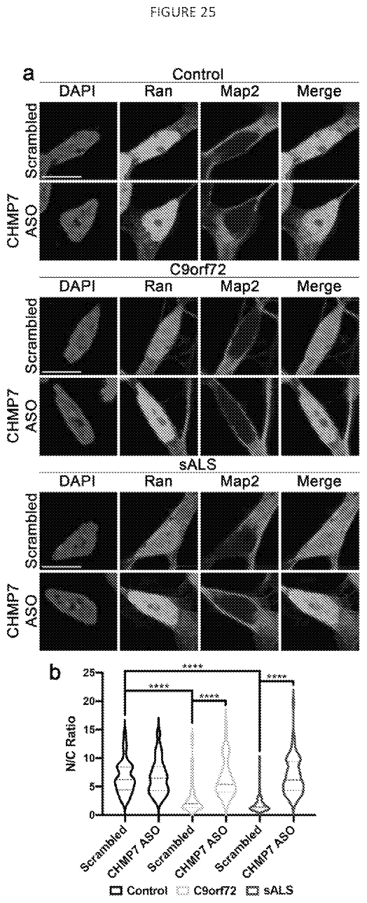

A critical function of the NPC is to govern nucleocytoplasmic transport (NCT). It is required that Ran GTPase is maintained at high levels within the nucleus in order to provide sufficient energy to power bidirectional NCT (46,77). As a result, the localization of Ran GTPase is often used as a static metric to evaluate alterations to NCT and multiple neurodegenerative disease models, including ALS/FTD iPSNs, display increased cytoplasmic Ran GTPase immunostaining (43, 78-80). It has previously been established that restoration of nuclear Nup levels significantly restores the localization of Ran GTPase to the nucleus in C9orf72 iPSNs (43). Consistent with this report, knockdown and CHMP7, and subsequent restoration of Nup expression within the nucleoplasm and NPC, restores the proper subcellular distribution of Ran GTPase in C9orf72 and sALS iPSNs (FIG. 25) as evaluated by immunostaining and confocal imaging.

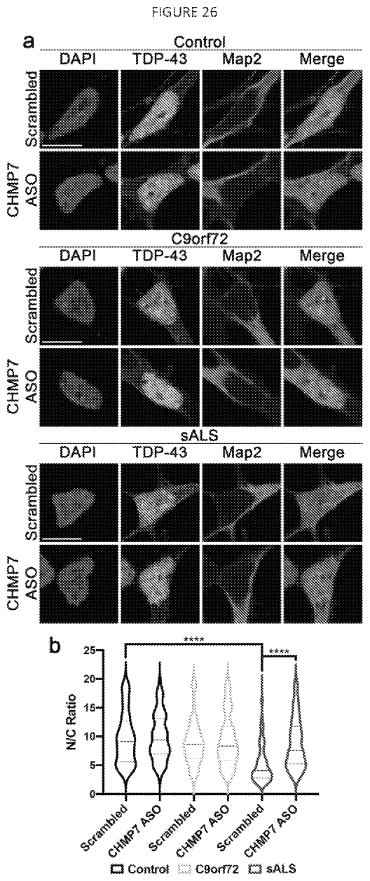

To determine whether knockdown of CHMP7 and the subsequent restoration of Nup levels within the nucleoplasm and NPCs of C9orf72 and sALS iPSN nuclei was sufficient to mitigate TDP-43 mediated splicing dysfunction, we performed qRT-PCR for full length and truncated stathmin-2 mRNAs. At day 46 of differentiation, about 2 weeks after the emergence of nuclear Nup reduction, a significant decrease in full length stathmin-2 mRNA was observed and a corresponding increase in truncated stathmin-2 mRNA in scrambled control ASO treated C9orf72 and sALS iPSNs (FIGS. 20G-20H). Following 3 week exposure to CHMP7 ASO 2, full length and truncated stathmin-2 mRNA levels were restored to levels observed in control iPSNs (FIG. 20G-20H). Further, in a subset of sALS iPSC lines evaluated, we observed a subtle but significant mislocalization of TDP-43 from the nucleus to the cytoplasm at day 46 of differentiation (FIG. 26). Consistent with a role in mitigating loss of TDP-43 function, 3-week treatment with CHMP7 ASO 2 restored nuclear TDP-43 localization in sALS iPSNs (FIG. 26).

Collectively, the observed Nup alterations are likely to impact multiple cellular functions and pathways critical to neuronal survival. As a result, we next evaluated the efficacy of CHMP7 ASOs in alleviating deficits in stressor induced neuronal survival. Two week treatment with CHMP7 ASO 2-significantly decreased the sensitivity of C9orf72 and sALS iPSNs to glutamate induced excitotoxicity (FIG. 20I) as measured by propidium iodide (PI) incorporation as previously described (41, 43). Together, these data highlight the therapeutic potential for CHMP7 ASOs in mitigating Nup and NPC defects, downstream TDP-43 dysfunction, and neuronal death in C9orf72 and sALS iPSNs.

### Example 14—CHMP7 Mediated Nup Alterations in Neurodegeneration

Pathological Nup alterations are prevalent in multiple neurodegenerative diseases including ALS, AD, and HD (78-81). However, the molecular mechanisms by which these disruptions occur remain poorly understood. In contrast to the Nup mislocalization observed in artificial overexpression mouse models of C9orf72 ALS/FTD (82-85), we have previously shown that in human neurons, there is a reduction in the nuclear levels of 8 specific Nups spanning multiple subcomplexes of the NPC without corresponding cytoplasmic accumulations (43). As a result, it was hypothesized that altered Nup surveillance and homeostasis may contribute to reduced Nup expression in C9orf72 and sALS iPSNs.

Recent work in yeast and non-neuronal cellular systems has identified CHMP7 as important mediator of NPC, Nup, and nuclear envelope homeostasis (48, 50, 51, 86). However, the contribution of this pathway to neuronal biology and disease pathophysiology remains unclear. Using an iPSN model of sporadic ALS and familial ALS/FTD, evidence is provided that disease associated increased nuclear CHMP7 expression contributes to reduced nuclear levels of key Nups located within 3 distinct domains of the NPC as an early event in disease pathogenesis. These data define a functional role for CHMP7 mediated Nup homeostasis in human neurons and neurodegeneration.

Although ESCRT-III mediated NPC and nuclear envelope repair is initiated by the nuclear specific ESCRT adapter protein CHMP7 (87), the recruitment and involvement of other CHMPs in this process remains unknown in multiple cellular systems including human neurons. Given that mutations in the ESCRT-III pathway component CHMP2B have been implicated in FTD (88), it will be interesting to evaluate whether or not these mutations are sufficient to induce nucleoporin reduction in a manner similar to that observed in C9orf72 and sALS iPSNs.

### Example 15—the Contribution of Nup Alterations to TDP-43 Pathology in Neurodegeneration

The nuclear clearance and mislocalization of TDP-43 has been regarded as a prominent pathological hallmark of ALS and related neurodegenerative diseases including AD and FTD (36, 72, 89). However, the biological events that may contribute to this pathology and/or loss of TDP-43 function in disease are poorly understood. Provided herein are multiple lines of evidence that nuclear retention of CHMP7 can at least in part contribute to TDP-43 mislocalization and dysfunction in disease. Interestingly, altered TDP-43 localization and deficits in stathmin-2 mRNA processing and TDP-43 mislocalization occur at time points well after the emergence of Nup alterations in C9orf72 and sALS iPSNs and control iPSNs artificially overexpressing a nuclear retained CHMP7. These data support our co-pathology analyses whereby 80-90% of postmortem disease neurons display increased CHMP7 immunoreactivity, but only a subset of those are also positive for TDP-43 pathology.

Collectively, this data suggests that increased nuclear CHMP7 is a critical initiator of pathogenic cascades impacting the nuclear levels of specific Nups spanning multiple subcomplexes of the neuronal NPC within C9orf72 and sALS nuclei. Ultimately, this combined “injury” to the NPC appears to contribute to TDP-43 dysfunction and downstream deficits in neuronal survival. Moreover, this data highlights CHMP7 as a potential therapeutic target in neurodegenerative diseases characterized by nuclear Nup reduction and TDP-43 pathology.

### Example 16

Materials and Methods

iPSC Derived Neuronal Differentiation

Mutant C9orf72, sALS, and non-neurological control iPSC lines were obtained from the Answer ALS repository at Cedars-Sinai (See Table L, below for demographics) and maintained on Matrigel with MTeSR according to Cedars Sinai standard operating procedures.

TABLE L
Demographic Information for iPSC Lines

| iPSC Line | | Clinical | Age at Time | | |
|---|---|---|---|---|---|
| Name | Source | Diagnosis | of Collection | Sex | Origin |
| EDi036-A | Cedars- | Non-neurologic | 79 | Female | PBMC |
| | Sinai | control | | | |
| EDi037-A | Cedars- | Non-neurologic | 79 | Male | PBMC |
| | Sinai | control | | | |
| EDi029-A | Cedars- | Non-neurologic | 80 | Male | PBMC |
| | Sinai | control | | | |
| EDi034-A | Cedars- | Non-neurologic | 79 | Female | PBMC |
| | Sinai | control | | | |
| CS1ATZ | Cedars- | Non-neurologic | 60 | Male | PBMC |
| | Sinai | control | | | |
| CS8PAA | Cedars- | Non-neurologic | 58 | Female | PBMC |
| | Sinai | control | | | |
| EDi043-A | Cedars- | Non-neurologic | 80 | Male | PBMC |
| | Sinai | control | | | |
| CS0002 | Cedars- | Non-neurologic | 51 | Male | PBMC |
| | Sinai | control | | | |
| CS9XH7 | Cedars- | Non-neurologic | 53 | Male | PBMC |
| | Sinai | control | | | |
| CS0BUU | Cedars- | C9orf72 ALS | 63 | Female | PMC |
| | Sinai | | | | |
| CS7VCZ | Cedars- | C9orf72 ALS | 64 | Male | PBMC |
| | Sinai | | | | |
| CS0LPK | Cedars- | C9orf72 ALS | 67 | Male | PBMC |
| | Sinai | | | | |
| CS6ZLD | Cedars- | C9orf72 ALS | | Female | PBMC |
| | Sinai | | | | |
| CS8KT3 | Cedars- | C9orf72 ALS | 60 | Male | PBMC |
| | Sinai | | | | |
| CS2YNL | Cedars- | C9orf72 ALS | 60 | Male | PBMC |
| | Sinai | | | | |
| CS0NKC | Cedars- | C9orf72 ALS | 52 | Female | PBMC |
| | Sinai | | | | |
| CS6CLW | Cedars- | C9orf72 ALS | | Male | PBMC |
| | Sinai | | | | |
| CS6UC9 | Cedars- | C9orf72 ALS | 54 | Male | PBMC |
| | Sinai | | | | |
| 59-1 | K. Talbot | Isogenic | 62 | Female | Fibro- |
| | | Correction of | | | blast |
| | | OXC9-02 | | | |
| OXC9-02- | K. Talbot | C9orf72 | 62 | Female | Fibro- |
| 02 | | | | | blast |
| CS3XLK | Cedars- | sALS | 55 | Female | PBMC |
| | Sinai | | | | |
| CS5JPF | Cedars- | sALS | 55 | Female | PBMC |
| | Sinai | | | | |
| CS8JGP | Cedars- | sALS | 61 | Male | PBMC |
| | Sinai | | | | |
| CS6MBU | Cedars- | sALS | | Male | PBMC |
| | Sinai | | | | |
| CS1KL3 | Cedars- | sALS | 71 | Female | PBMC |
| | Sinai | | | | |
| CS9GXD | Cedars- | sALS | 68 | Male | PBMC |
| | Sinai | | | | |
| CS0JGZ | Cedars- | sALS | 56 | Male | PBMC |
| | Sinai | | | | |
| CS2EVP | Cedars- | sALS | 69 | Male | PBMC |
| | Sinai | | | | |
| CS6PYD | Cedars- | sALS | 69 | Female | PBMC |
| | Sinai | | | | |
| CS5ZHY | Cedars- | sALS | 64 | Male | PBMC |
| | Sinai | | | | |

iPSCs were differentiated into mixed spinal neuronal populations using the direct induced motor neuron (diMNs) protocol as previously described in Coyne A N, Baskerville V, Zaepfel B L, Dickson D W, Rigo F, Bennett F, Patrick Lusk C, Rothstein J D, “Nuclear accumulation of CHMP7 initiates nuclear pore complex injury and subsequent TDP-43 dysfunction in sporadic and familial ALS”, Sci Translat. Med. (2021) and Coyne A N, Zaepfel B L, Hayes L, Fitchman B, Salzberg Y, Luo E C, Bowen K, Trost H, Aigner S, Rigo F et al. “G(4)C(2) repeat RNA initiates a POM121-mediated reduction in specific nucleoporins in C9orf72 ALS/FTD, Neuron (2020). All cells were maintained at 37° C. with 5% CO₂. iPSCs and iPSNs routinely tested negative for mycoplasma.

ASO Treatment of iPSNs

As previously described in Coyne A N, Baskerville V, Zaepfel B L, Dickson D W, Rigo F, Bennett F, Patrick Lusk C, Rothstein J D, “Nuclear accumulation of CHMP7 initiates nuclear pore complex injury and subsequent TDP-43 dysfunction in sporadic and familial ALS”, Sci Translat. Med. (2021), on day 25 of differentiation, 5 μM scrambled control (676630): CCTATAGGACTATCCAGGAA (SEQ ID NO: 2) or CHMP7 targeting (1508917): TGTTACCCTCAGATACCGCC (SEQ ID NO:4) ASOs were added to the culture media. Media and ASO were exchanged every 3 days until iPSN analyses were carried out on day 40 of differentiation. ASOs were generously provided by Ionis Pharmaceuticals.

Nucleofection of iPSNs

On day 18 of differentiation, iPSNs were dissociated with Accutase following manufacturer protocol to assist with single cell dissociated and subjected to suspension based nucleofection using the Lonza P3 Primary Cell 4D Nucleofector Kit (Lonza) and program DC104. 5 million iPSNs and 4 μg plasmid DNA were used for each nucleofection reaction. Plasmids used are as follows: GFP (Addgene 54759), VPS4 GFP (Addgene 116924), and VPS4^{E228Q} GFP (Addgene 80351). Nucleofected iPSNs were plated in Matrigel (Corning) coated cell culture dishes and media was exchanged the next day and subsequently every 3 days until downstream analyses on day 40 of differentiation.

Nuclei Isolation and Super Resolution Structured Illumination Microscopy

Nuclei were isolated from iPSNs and postmortem human motor and occipital cortex tissue using the Nuclei Pure Prep Nuclei Isolation Kit (Sigma Aldrich) following manufacturer protocol with slight modifications as previously described in Coyne A N, Baskerville V, Zaepfel B L, Dickson D W, Rigo F, Bennett F, Patrick Lusk C, Rothstein J D, “Nuclear accumulation of CHMP7 initiates nuclear pore complex injury and subsequent TDP-43 dysfunction in sporadic and familial ALS”, Sci Translat. Med. (2021) and Coyne A N, Zaepfel B L, Hayes L, Fitchman B, Salzberg Y, Luo E C, Bowen K, Trost H, Aigner S, Rigo F et al. “G(4)C(2) repeat RNA initiates a POM121-mediated reduction in specific nucleoporins in C9orf72 ALS/FTD, Neuron (2020). About 10 million iPSNs or 100 mg of frozen postmortem motor cortex tissue (obtained from the Target ALS Human Postmortem Tissue Core (see Table M for demographics) was used for nuclei isolation.

TABLE M
Demographic Information for Postmortem Human Tissue

| | Clinical Diagnosis | Age of Death | Sex |
|---|---|---|---|
| Control | Non-neurologic control | 70 | Female |
| Control | Non-neurologic control | 92 | Female |
| Control | Non-neurologic control | 72 | Male |
| Control | Non-neurologic control | 37 | Female |
| Control | Non-neurologic control | 50 | Male |
| Control | Non-neurologic control | 52 | Male |
| C9orf72 | C9orf72 ALS/FTD | 59 | Male |
| C9orf72 | C9orf72 ALS | 72 | Male |
| C9orf72 | C9orf72 ALS | 69 | Female |
| C9orf72 | C9orf72 ALS/FTD | 61 | Female |
| C9orf72 | C9orf72 ALS | 68 | Female |
| C9orf72 | C9orf72 ALS/FTD | 74 | Male |
| sALS-1 | sALS | 69 | Male |
| sALS-2 | sALS | 68 | Female |
| sALS-3 | sALS | 69 | Male |
| sALS-4 | sALS | 67 | Female |
| sALS-5 | sALS | 59 | Female |
| sALS-6 | sALS | 68 | Male |
| sALS-7 | sALS | 71 | Female |
| sALS-8 | sALS | 66 | Male |
| sALS-9 | sALS | 70 | Female |

A 1.85 M sucrose gradient was used to enrich for neuronal nuclei. Following isolation, nuclei were centrifuged onto collagen coated (1 mg/mL; Advanced Biomatrix) slides with a CytoSpin 4 centrifuge (Thermo Fisher Scientific) and immunostained as previously described in Coyne A N, Baskerville V, Zaepfel B L, Dickson D W, Rigo F, Bennett F, Patrick Lusk C, Rothstein J D, “Nuclear accumulation of CHMP7 initiates nuclear pore complex injury and subsequent TDP-43 dysfunction in sporadic and familial ALS”, Sci Translat. Med. (2021) and Coyne A N, Zaepfel B L, Hayes L, Fitchman B, Salzberg Y, Luo E C, Bowen K, Trost H, Aigner S, Rigo F et al. “G(4)C(2) repeat RNA initiates a POM121-mediated reduction in specific nucleoporins in C9orf72 ALS/FTD, Neuron (2020) (see Table N, below).

TABLE N
Antibody Information
Primary Antibodies

| | | Catalog | Application and |
|---|---|---|---|
| Antibody | Source | Number | Concentration |
| Rabbit Anti-CHMP4B | Proteintech | 13683-1-AP | IF: 1/250 |
| | | | Western: 1/1000 |
| Rabbit Anti-CHMP2B | Thermo Fisher | PA531128 | IF: 1/250 |
| | Scientific | | Western: 1/1000 |
| Mouse Anti-VPS4 | Santa Cruz | sc-133122 | IF: 1/50 |
| | Biotechnology | | Western: 1/250 |
| Rabbit Anti-POM121 | Novus | NBP2-19890 | IF: 1/250 |
| | Biologicals | | |
| Chicken Anti-NeuN | Millipore | ABN91 | IF: 1/500 |
| Chicken Anti-GFP | Millipore | AB16901 | IF: 1/1000 |
| Rabbit Anti-Lamin B1 | Abcam | ab16048 | Western: 1/1000 |
| Mouse Anti-GAPDH | Life | AM4300 | Western: |
| | Technologies | | |
| | | | 1/10,000 |

Secondary Antibodies

| | | | |
|---|---|---|---|
| Goat Anti-Mouse Alexa | Invitrogen | A11029 | IF: 1/1000 |
| 488 | | | |
| Goat Anti-Rabbit Alexa | Invitrogen | A11034 | IF: 1/1000 |
| 488 | | | |
| Goat Anti-Chicken | Invitrogen | A11039 | IF: 1/1000 |
| Alexa 488 | | | |
| Goat Anti-Mouse Alexa | Invitrogen | A11031 | IF: 1/1000 |
| 568 | | | |
| Goat Anti-Rabbit Alexa | Invitrogen | A11036 | IF: 1/1000 |
| 568 | | | |
| Goat Anti-Chicken | Invitrogen | A21449 | IF: 1/1000 |
| Alexa 647 | | | |
| Donkey Anti-Rabbit | Thermo Fisher | 45-000-682 | Western: 1/5000 |
| IgG HRP | Scientific | | |
| Horse Anti-Mouse IgG | Cell Signaling | 7076S | Western: 1/5000 |
| HRP | | | |

Isolated nuclei were subsequently imaged by super resolution structured illumination microscopy (SIM) using a Zeiss ELYRA Si as previously described in Coyne A N, Baskerville V, Zaepfel B L, Dickson D W, Rigo F, Bennett F, Patrick Lusk C, Rothstein J D, “Nuclear accumulation of CHMP7 initiates nuclear pore complex injury and subsequent TDP-43 dysfunction in sporadic and familial ALS”, Sci Translat. Med. (2021) and Coyne A N, Zaepfel B L, Hayes L, Fitchman B, Salzberg Y, Luo E C, Bowen K, Trost H, Aigner S, Rigo F et al. “G(4)C(2) repeat RNA initiates a POM121-mediated reduction in specific nucleoporins in C9orf72 ALS/FTD, Neuron (2020). All images were acquired using identical imaging parameters (e.g. laser power, gain) and subjected to default SIM deconvolution and processing in Zeiss Zen Black 2.3 SP1. Representative images are presented as 3D maximum intensity projections generated in Zeiss Zen Black 2.3 SP1. Images were faux colored green for contrast and display.

Western Blots

Nuclei and iPSN lysates were generated as previously described in Coyne A N, Baskerville V, Zaepfel B L, Dickson D W, Rigo F, Bennett F, Patrick Lusk C, Rothstein J D, “Nuclear accumulation of CHMP7 initiates nuclear pore complex injury and subsequent TDP-43 dysfunction in sporadic and familial ALS”, Sci Translat. Med. (2021) and Coyne A N, Zaepfel B L, Hayes L, Fitchman B, Salzberg Y, Luo E C, Bowen K, Trost H, Aigner S, Rigo F et al. “G(4)C(2) repeat RNA initiates a POM121-mediated reduction in specific nucleoporins in C9orf72 ALS/FTD, Neuron (2020) using RIPA lysis buffer. 5 μg protein was subjected to SDS-PAGE using 4-20% acrylamide gels (BioRad) and transferred onto nitrocellulose membranes with the Trans-Blot Turbo Transfer System as previously described in Coyne A N, Baskerville V, Zaepfel B L, Dickson D W, Rigo F, Bennett F, Patrick Lusk C, Rothstein J D, “Nuclear accumulation of CHMP7 initiates nuclear pore complex injury and subsequent TDP-43 dysfunction in sporadic and familial ALS”, Sci Translat. Med. (2021) and Coyne A N, Zaepfel B L, Hayes L, Fitchman B, Salzberg Y, Luo E C, Bowen K, Trost H, Aigner S, Rigo F et al. “G(4)C(2) repeat RNA initiates a POM121-mediated reduction in specific nucleoporins in C9orf72 ALS/FTD, Neuron (2020). Following 30 minutes room temperature incubation in block (5% nonfat milk in 1×TBS containing 0.1% Tween-20), blots were incubated with rotation in primary antibody (See, Table N) diluted in block overnight at 4° C. After 16-18 hours, blots were washed 4×10 minutes with 1×TBST and then incubated with rotation in secondary antibody (See, Table N) diluted in block for 1 hour at room temperature. Blots wee then washed 4×10 minutes with 1×TBST and incubated with ECL substrate (Thermo Fisher Scientific, Millipore) for 30 seconds. The GE Healthcare ImageQuant LAS 400 system was used to acquire chemiluminescent images. Blots were incubated for 15 minutes at room temperature with 30% H₂O₂ to facilitate sequential probing without stripping (Sennepin A D, Charpentier S, Normand T, Sarre C, Legrand A, Mollet L M, Multiple reprobing of Western blots after inactivation of peroxidase activity by its substrate, hydrogen peroxide, Anal Biochem, 393:129-131 (2009)). Analysis was carried out with FIJI software. GAPDH and Lamin B1 were used for normalization.

Statistical Analysis

All data analysis was conducted with FIJI or Imaris as previously described in Coyne A N, Baskerville V, Zaepfel B L, Dickson D W, Rigo F, Bennett F, Patrick Lusk C, Rothstein J D, “Nuclear accumulation of CHMP7 initiates nuclear pore complex injury and subsequent TDP-43 dysfunction in sporadic and familial ALS”, Sci Translat. Med. (2021) and Coyne A N, Zaepfel B L, Hayes L, Fitchman B, Salzberg Y, Luo E C, Bowen K, Trost H, Aigner S, Rigo F et al. “G(4)C(2) repeat RNA initiates a POM121-mediated reduction in specific nucleoporins in C9orf72 ALS/FTD, Neuron (2020). The analyzer was completely blinded to genotype/treatment/time point/brain region information. All statistical analyses were performed using Prism version 9 (GraphPad). For imaging experiments, statistical analyses were performed whereby the average of all nuclei or cells evaluated per each iPSC line, patient, and treatment condition represents n=1. The total number of nuclei or cells evaluated per experiment is indicated in the description of the figures. Two-tailed Student's t-test, One-way ANOVA with Tukey's multiple comparison test, or Two-way ANOVA with Tukey's multiple comparison test was used as appropriate for experimental design and as indicated in the description of the figures. *p<0.05, **p<0.01, ***p<0.001, ****p<0.0001. Violin plots are used to display the full spread and variability of large data sets (>10 data points). Center dotted line indicates median value. Two additional dotted lines indicate the 25th and 75th percentiles. Bar graphs with individual data points are used to display summary data sets with <10 data points.

Results

Expression of the ESCRT-III protein VPS4 is increased in C9orf72 and sALS neuronal nuclei

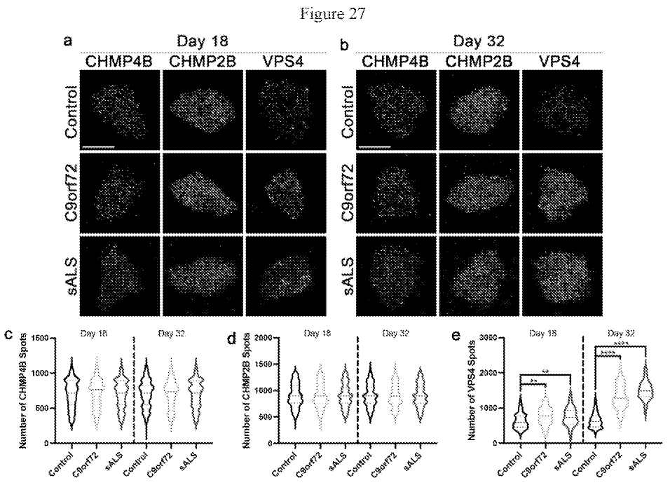

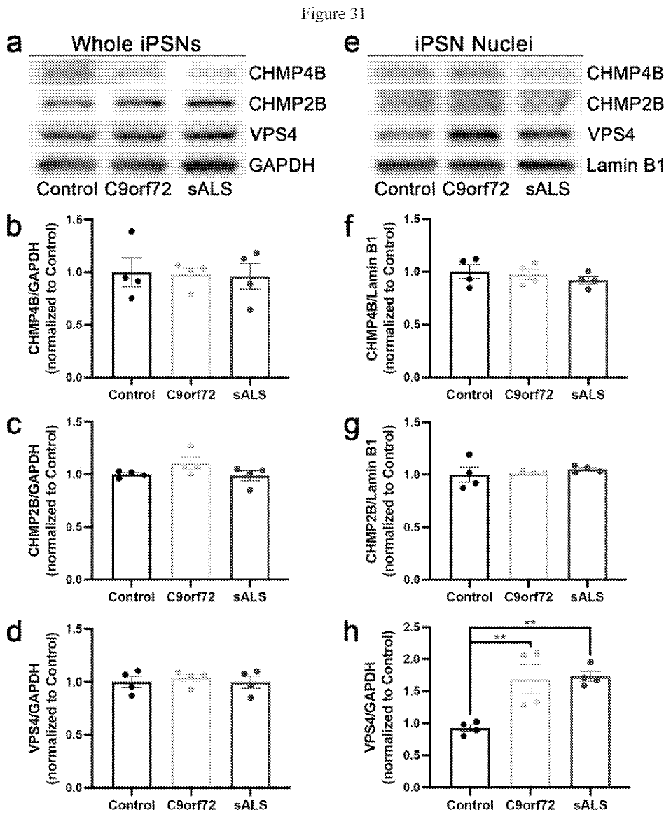

Increased nuclear localization and expression of the ESCRT-III protein CHMP7 was previously identified as an early and consequential pathologic event leading to NPC injury in C9orf72 ALS/FTD and sALS (Coyne A N, Baskerville V, Zaepfel B L, Dickson D W, Rigo F, Bennett F, Patrick Lusk C, Rothstein J D, “Nuclear accumulation of CHMP7 initiates nuclear pore complex injury and subsequent TDP-43 dysfunction in sporadic and familial ALS”, Sci Translat. Med. (2021)). Given the previously reported involvement of additional ESCRT-III proteins (CHMP4B, CHMP2B, and VPS4) in NPC and NE surveillance and homeostasis (Denais C M, Gilbert R M, Isermann P, McGregor A L, to Lindert M, Weigelin B, Davidson P M, Friedl P, Wolf K, Lammerding J, Nuclear envelope rupture and repair during cancer cell migration, Science (New York, NY) 352:353-358, (2016), Toyama B H, Arrojo E D R, Lev-Ram V, Ramachandra R, Deerinck T J, Lechene C, Ellisman M H, Hetzer M W, Visualization of long-lived proteins reveals age mosaicism within nuclei of postmitotic cells, J Cell Biol. (2018), Vietri M, Radulovic M, Stenmark H, The many functions of ESCRTs, Nat Rev Mol Cell Biol. (2019), Vietri M, Schultz S W, Bellanger A, Jones C M, Petersen L I, Raiborg C, Skarpen E, Pedurupillay C R J, Kjos I, Kip E et al, Unrestrained ESCRT-III drives micronuclear catastrophe and chromosome fragmentation, Nat Cell Biol., 22:856-867 (2020)), an examination was conducted to determine whether the nuclear distribution and cellular expression of CHMP4B, CHMP2B, and VPS4 was altered in ALS neuronal nuclei. Using immunostaining SIM, it was found that the amount of VPS4, but not CHMP4B nor CHMP2B, was increased in nuclei isolated from C9orf72 ALS/FTD and sALS iPSNs compared to controls (FIG. 27). Similar to the published observations for CHMP7 (Coyne A N, Baskerville V, Zaepfel B L, Dickson D W, Rigo F, Bennett F, Patrick Lusk C, Rothstein J D, “Nuclear accumulation of CHMP7 initiates nuclear pore complex injury and subsequent TDP-43 dysfunction in sporadic and familial ALS”, Sci Translat. Med. (2021)), the increase in nuclear VPS4 spots occurred at a time point prior to the initiation of NPC injury (FIG. 27A, FIG. 27E). Importantly, an overall increase in VPS4, CHMP4B, or CHMP2B levels in C9orf72 ALS/FTD or sALS whole cell iPSN lysates was not detected (FIGS. 31A-31D). Highlighting the utility of SIM for evaluating not only protein distribution, but also expression, western blot analyses quantitatively confirmed an increase in VPS4, but not CHMP4B nor CHMP2B protein in nuclei isolated from C9orf72 ALS/FTD and sALS iPSNs (FIGS. 31E-31H). Together, these data suggest that like CHMP7 (Coyne A N, Baskerville V, Zaepfel B L, Dickson D W, Rigo F, Bennett F, Patrick Lusk C, Rothstein J D, “Nuclear accumulation of CHMP7 initiates nuclear pore complex injury and subsequent TDP-43 dysfunction in sporadic and familial ALS”, Sci Translat. Med. (2021)), VPS4 may be relocalized from the cytoplasm to the nucleus in ALS neurons.

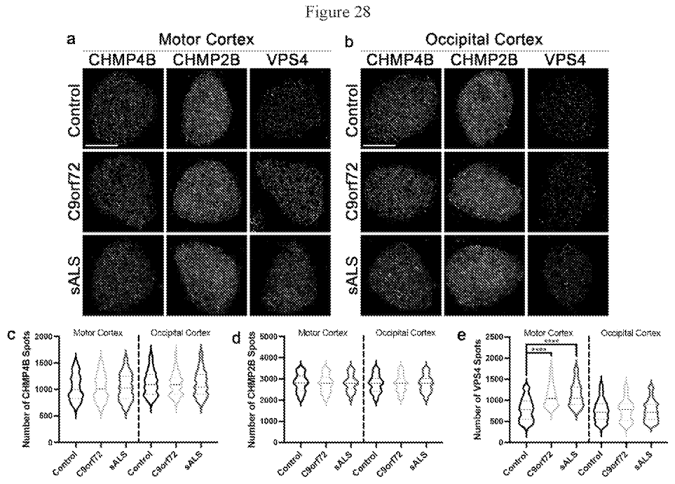

To validate that the results from iPSNs recapitulates observations in real human disease tissues, an immunostaining and SIM for CHMP4B, CHMP2B, and VPS4 was performed in nuclei isolated from postmortem human motor and occipital cortex tissues. Consistent with the data in iPSNs, VPS4, but not CHMP4B nor CHMP2B, was increased in neuronal nuclei from C9orf72 ALS/FTD and sALS motor cortex (FIG. 28A, FIGS. 28C-28E). In contrast, in the occipital cortex, a control brain region unaffected in ALS, no change in the expression of CHMP4B, CHMP2B, or VPS4 in C9orf72 ALS/FTD or sALS neuronal nuclei was observed (FIGS. 28B-28E).

ASO Mediated Knockdown of CHMP7 Mitigates VPS4 Pathology in C9orf72 and sALS Neuronal Nuclei

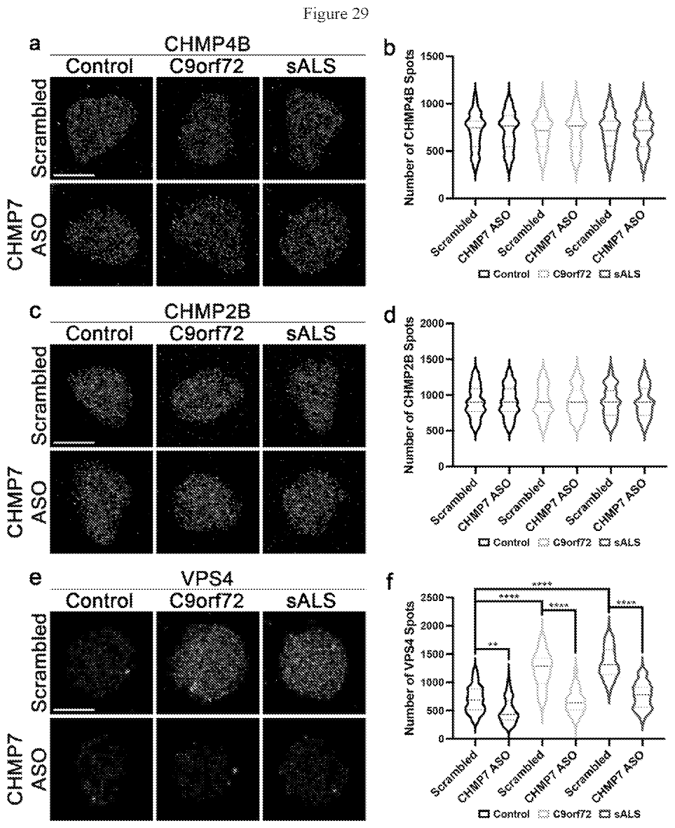

The nuclear relocalization of CHMP7 from the cytoplasm to the nucleus has been proposed to initiate the recruitment of additional ESCRT-III pathway components culminating in scission and removal of NPC and NE components by VPS4 (Denais C M, Gilbert R M, Isermann P, McGregor A L, to Lindert M, Weigelin B, Davidson P M, Friedl P, Wolf K, Lammerding J, Nuclear envelope rupture and repair during cancer cell migration, Science (New York, NY) 352:353-358 (2016), Thaller D J, Allegretti M, Borah S, Ronchi P, Beck M, Lusk C P, An ESCRT-LEM protein surveillance system is poised to directly monitor the nuclear envelope and nuclear transport system, Elife (2019), Toyama B H, Arrojo E D R, Lev-Ram V, Ramachandra R, Deerinck T J, Lechene C, Ellisman M H, Hetzer M W Visualization of long-lived proteins reveals age mosaicism within nuclei of postmitotic cells, J Cell Biol., (2018), Vietri M, Radulovic M, Stenmark H, The many functions of ESCRTs, Nat Rev Mol Cell Biol. (2019), Vietri M, Schultz S W, Bellanger A, Jones C M, Petersen L I, Raiborg C, Skarpen E, Pedurupillay C R J, Kjos I, Kip E et al., Unrestrained ESCRT-III drives micronuclear catastrophe and chromosome fragmentation, Nat Cell Biol., 22:856-867 (2020), Webster B M, Colombi P, Jager J, Lusk C P, Surveillance of nuclear pore complex assembly by ESCRT-III/Vps4, Cell 159:388-401 (2014), Webster B M, Thaller D J, Jager J, Ochmann S E, Borah S, Lusk C P, Chm7 and Heh1 collaborate to link nuclear pore complex quality control with nuclear envelope sealing, EMBO J, 35:2447-2467 (2016)). It has not previously been shown that antisense oligonucleotide (ASO) mediated knockdown of CHMP7 not only alleviates the aberrant nuclear accumulation of CHMP7, but also robustly mitigates NPC injury and downstream NPC and TDP-43 dysfunction in C9orf72 and sALS iPSNs (Coyne AN, Baskerville V, Zaepfel B L, Dickson D W, Rigo F, Bennett F, Patrick Lusk C, Rothstein J D, “Nuclear accumulation of CHMP7 initiates nuclear pore complex injury and subsequent TDP-43 dysfunction in sporadic and familial ALS”, Sci Translat. Med. (2021)). To test whether increased nuclear CHMP7 impacted the nuclear distribution and expression of downstream components of the ESCRT-III pathway in human neurons, a control and ALS iPSNs with CHMP7 targeting ASOs (Coyne A N, Baskerville V, Zaepfel B L, Dickson D W, Rigo F, Bennett F, Patrick Lusk C, Rothstein J D, “Nuclear accumulation of CHMP7 initiates nuclear pore complex injury and subsequent TDP-43 dysfunction in sporadic and familial ALS”, Sci Translat. Med. (2021)) was treated for 2 weeks after the emergence of CHMP7 (Coyne A N, Baskerville V, Zaepfel B L, Dickson D W, Rigo F, Bennett F, Patrick Lusk C, Rothstein J D, “Nuclear accumulation of CHMP7 initiates nuclear pore complex injury and subsequent TDP-43 dysfunction in sporadic and familial ALS”, Sci Translat. Med. (2021)) and VPS4 (FIG. 27a, 27d) pathology (see Materials and Methods, above). Using SIM, it was found that ASO mediated CHMP7 knockdown has no impact on nuclear CHMP4B and CHMP2B immunoreactivity (FIGS. 29A-29D). In contrast, reduction of CHMP7 levels (See, Coyne A N, Baskerville V, Zaepfel B L, Dickson D W, Rigo F, Bennett F, Patrick Lusk C, Rothstein J D, “Nuclear accumulation of CHMP7 initiates nuclear pore complex injury and subsequent TDP-43 dysfunction in sporadic and familial ALS”, Sci Translat. Med. (2021)) significantly decreases nuclear VPS4 immunoreactivity (FIG. 29E, 29F) suggesting that increased nuclear VPS4 expression is dependent on CHMP7.

Impaired VPS4 Function is not Sufficient to Alleviate NPC Alterations in C9orf72 and sALS Neuronal Nuclei

It has previously been reported that substantial reduction of the Nup POM121 from NPC and nucleoplasm is an early and consistent injury to the NPC (Coyne A N, Zaepfel B L, Hayes L, Fitchman B, Salzberg Y, Luo E C, Bowen K, Trost H, Aigner S, Rigo F et al. “G(4)C(2) repeat RNA initiates a POM121-mediated reduction in specific nucleoporins in C9orf72 ALS/FTD, Neuron (2020)) as a result of aberrant nuclear accumulation of CHMP7 (Coyne A N, Baskerville V, Zaepfel B L, Dickson D W, Rigo F, Bennett F, Patrick Lusk C, Rothstein J D, “Nuclear accumulation of CHMP7 initiates nuclear pore complex injury and subsequent TDP-43 dysfunction in sporadic and familial ALS”, Sci Translat. Med. (2021)). As VPS4 is a AAA-ATPase that facilitates the removal of NPC and NE components from the nucleus and nuclear membrane (Thaller D J, Allegretti M, Borah S, Ronchi P, Beck M, Lusk C P, Vietri M, Radulovic M, Stenmark H, The many functions of ESCRTs, Nat Rev Mol Cell Biol. (2019), Webster B M, Colombi P, Jager J, Lusk C P, Surveillance of nuclear pore complex assembly by ESCRT-III/Vps4, Cell, 159:388-401 (2014), An ESCRT-LEM protein surveillance system is poised to directly monitor the nuclear envelope and nuclear transport system, Elife (2019), Webster B M, Lusk C P, Border safety: quality control at the nuclear envelope, Trends Cell Biol., 26:29-39 (2016), Webster B M, Thaller D J, Jager J, Ochmann S E, Borah S, Lusk C P, Chm7 and Heh1 collaborate to link nuclear pore complex quality control with nuclear envelope sealing, EMBO J, 35:2447-246 (2016)), it was hypothesized that increased nuclear VPS4 expression might also functionally contribute to NPC injury in C9orf72 ALS/FTD and sALS neurons. To test this, GFP tagged wildtype VPS4 or a GFP tagged dominant negative variant of VPS4 (VPS4^{E228Q}) that has previously been shown to impair ESCRT dependent release events (Votteler J, Ogohara C, Yi S, Hsia Y, Nattermann U, Belnap D M, King N P, Sundquist W I, Designed proteins induce the formation of nanocage-containing extracellular vesicles, Nature, 540:292-295 (2016)) was overexpressed and SIM and immunostaining performed for POM121 in nuclei isolated from control and ALS iPSNs.

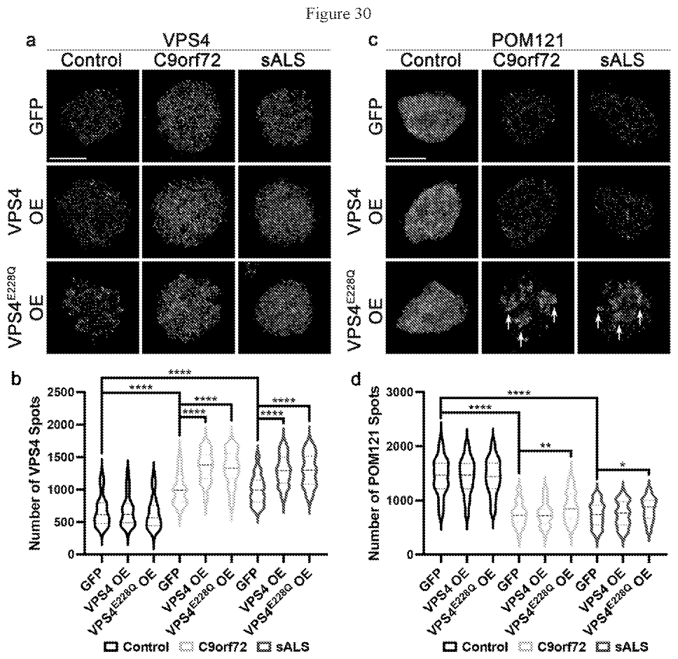

Overexpression of VPS4 variants increased the nuclear expression of VPS4 as detected by immunostaining in C9orf72 and sALS, but not control iPSNs (FIG. 30A, FIG. 30B) suggesting that nuclear recruitment of VPS4 is not “hyper activated” in the context of a wildtype human neuron. Consistent with a function for VPS4 downstream of CHMP7, VPS4^{E228Q} overexpression only partially restored the nuclear expression of POM121 in C9orf72 and sALS iPSNs (FIG. 30C, FIG. 30D). Intriguingly, overexpression of wildtype VPS4 had no impact on nuclear POM121 immunoreactivity in C9orf72 and sALS iPSNs (FIG. 30c, FIG. 30d) suggesting that simply increasing nuclear VPS4 levels is not sufficient to enhance NPC injury. Compared to control nuclei, the distribution of POM121 appears to be abnormal in C9orf72 ALS/FTD and sALS nuclei overexpressing VPS4^{E228Q} (FIG. 30C) for reasons that remain unclear. Notably, Trim21 mediated knockdown (Trim Away, (Clift D, McEwan W A, Labzin L I, Konieczny V, Mogessie B, James L C, Schuh M, A method for the acute and rapid degradation of endogenous proteins, Cell 171:1692-1706.e1618 (2017)) of endogenous VPS4 protein was toxic to iPSNs perhaps as a result of its functions beyond nuclear envelope and NPC homeostasis (McCullough J, Frost A, Sundquist W I, Structures, functions, and dynamics of ESCRT-III/Vps4 membrane remodeling and fission complexes, Annu Rev Cell Dev Biol., 34:85-109 (2018), Vietri M, Radulovic M, Stenmark H, The many functions of ESCRTs, Nat Rev Mol Cell Biol., (2019)). Collectively, these data suggest that in contrast to CHMP7 knockdown (Coyne A N, Baskerville V, Zaepfel B L, Dickson D W, Rigo F, Bennett F, Patrick Lusk C, Rothstein J D, “Nuclear accumulation of CHMP7 initiates nuclear pore complex injury and subsequent TDP-43 dysfunction in sporadic and familial ALS”, Sci Translat. Med. (2021)), impaired VPS4 function is not sufficient to restore the expression and distribution of specific Nups within C9orf72 ALS/FTD and sALS nuclei.

Nuclear pore complex injury, pathological cytoplasmic accumulations of specific Nups, and defects and nucleocytoplasmic transport have now been reported as prominent pathological features of multiple neurodegenerative diseases including ALS/FTD, AD, and HD (in Coyne A N, Baskerville V, Zaepfel B L, Dickson D W, Rigo F, Bennett F, Patrick Lusk C, Rothstein J D, “Nuclear accumulation of CHMP7 initiates nuclear pore complex injury and subsequent TDP-43 dysfunction in sporadic and familial ALS”, Sci Transla.t Med. (2021), Coyne A N, Zaepfel B L, Hayes L, Fitchman B, Salzberg Y, Luo E C, Bowen K, Trost H, Aigner S, Rigo F et al. “G(4)C(2) repeat RNA initiates a POM121-mediated reduction in specific nucleoporins in C9orf72 ALS/FTD, Neuron (2020), Eftekharzadeh B, Daigle J G, Kapinos L E, Coyne A, Schiantarelli J, Carlomagno Y, Cook C, Miller S J, Dujardin S, Amaral A S et al., Tau protein disrupts nucleocytoplasmic transport in Alzheimer's disease, Neuron 99:925-940.e927 (2018), Grima J C, Daigle J G, Arbez N, Cunningham K C, Zhang K, Ochaba J, Geater C, Morozko E, Stocksdale J, Glatzer J C et al., Mutant huntingtin disrupts the nuclear pore complex, Neuron 94:93-107.e106 (2017), and Zhang K, Donnelly C J, Haeusler A R, Grima J C, Machamer J B, Steinwald P, Daley E L, Miller S J, Cunningham K M, Vidensky S et al., The C9orf72 repeat expansion disrupts nucleocytoplasmic transport, Nature 525:56-61 (2015). However, the molecular mechanisms leading to these disruptions are still poorly understood. It has recently been established that aberrant nuclear accumulation of the ESCRT-III protein CHMP7 is sufficient to initiate a reduction in specific Nups, beginning with POM121, from the NPC and nucleoplasm of C9orf72 ALS/FTD and sALS human neuronal nuclei (Coyne A N, Baskerville V, Zaepfel B L, Dickson D W, Rigo F, Bennett F, Patrick Lusk C, Rothstein J D, “Nuclear accumulation of CHMP7 initiates nuclear pore complex injury and subsequent TDP-43 dysfunction in sporadic and familial ALS”, Sci Translat. Med. (2021)). Ultimately, this nuclear pore injury impacts NCT and subsequent TDP-43 function and localization and downstream neuronal survival in response to glutamate stress (Coyne A N, Zaepfel B L, Hayes L, Fitchman B, Salzberg Y, Luo E C, Bowen K, Trost H, Aigner S, Rigo F et al. “G(4)C(2) repeat RNA initiates a POM121-mediated reduction in specific nucleoporins in C9orf72 ALS/FTD, Neuron (2020)). To further characterize ESCRT-III protein pathology and the contribution to NPC injury in familial and sporadic ALS, this study shows that VPS4, but not CHMP4B or CHMP2B, is increased in a CHMP7 dependent manner in C9orf72 ALS/FTD and sALS human neuronal nuclei. Together, this data supports a critical role for and highlight the complexity of the ESCRT-III pathway in NPC injury in ALS/FTD. All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

### REFERENCES

1. K. Zhang et al., The C9orf72 repeat expansion disrupts nucleocytoplasmic transport. Nature 525, 56-61 (2015).
2. J. Chew et al., Aberrant deposition of stress granule-resident proteins linked to C9orf72-associated TDP-43 proteinopathy. Molecular neurodegeneration 14, 9 (2019).
3. Y. J. Zhang et al., Poly(GR) impairs protein translation and stress granule dynamics in C9orf72-associated frontotemporal dementia and amyotrophic lateral sclerosis. Nature medicine, (2018).
4. Y. J. Zhang et al., C9ORF72 poly(GA) aggregates sequester and impair HR23 and nucleocytoplasmic transport proteins. Nature neuroscience 19, 668-677 (2016).
5. Y. J. Zhang et al., Heterochromatin anomalies and double-stranded RNA accumulation underlie C9orf72 poly(PR) toxicity. Science (New York, N.Y.) 363, (2019).
6. A. N. Coyne et al., G(4)C(2) Repeat RNA Initiates a POM121-Mediated Reduction in Specific Nucleoporins in C9orf72 ALS/FTD. Neuron, (2020).
7. B. M. Webster, P. Colombi, J. Jager, C. P. Lusk, Surveillance of nuclear pore complex assembly by ESCRT-III/Vps4. Cell 159, 388-401 (2014).
8. D. J. Thaller et al., An ESCRT-LEM protein surveillance system is poised to directly monitor the nuclear envelope and nuclear transport system. eLife 8, (2019).
9. B. M. Webster et al., Chm7 and Heh1 collaborate to link nuclear pore complex quality control with nuclear envelope sealing. The EMBO journal 35, 2447-2467 (2016).
10. B H. Toyama et al., Visualization of long-lived proteins reveals age mosaicism within nuclei of postmitotic cells. The Journal of cell biology, (2018).
11. D. Clift et al., A Method for the Acute and Rapid Degradation of Endogenous Proteins. Cell 171, 1692-1706.e1618 (2017).
12. S. Mizielinska et al., C9orf72 repeat expansions cause neurodegeneration in Drosophila through arginine-rich proteins. Science (New York, N.Y.) 345, 1192-1194 (2014).
13. M. Vietri, M. Radulovic, H. Stenmark, The many functions of ESCRTs. Nature reviews. Molecular cell biology, (2019).
14. S. L. DeVos, T. M. Miller, Antisense oligonucleotides: treating neurodegeneration at the level of RNA. Neurotherapeutics: the journal of the American Society for Experimental Neuro Therapeutics 10, 486-497 (2013).
15. K. M. Schoch, T. M. Miller, Antisense Oligonucleotides: Translation from Mouse Models to Human Neurodegenerative Diseases. Neuron 94, 1056-1070 (2017).
16. C. J. Donnelly et al., RNA toxicity from the ALS/FTD C9ORF72 expansion is mitigated by antisense intervention. Neuron 80, 415-428 (2013).
17. M. Gu et al., LEM2 recruits CHMP7 for ESCRT-mediated nuclear envelope closure in fission yeast and human cells. Proceedings of the National Academy of Sciences of the United States of America 114, E2166-e2175 (2017).
18. M. Vietri et al., Unrestrained ESCRT-III drives micronuclear catastrophe and chromosome fragmentation. Nature cell biology 22, 856-867 (2020).
19. Kudo et al., Leptomycin B inactivates CRM1/exportin 1 by covalent modification at a cysteine residue in the central conserved region. Proceedings of the National Academy of Sciences of the United States of America 96, 9112-9117 (1999).
20. D. J. Thaller et al., Direct PA-binding by Chm7 is required for nuclear envelope surveillance at herniations. bioRxiv, 2020.2005.2004.074880 (2020).
21. M. Delaleau, K. L. Borden, Multiple Export Mechanisms for mRNAs. Cells 4, 452-473 (2015).
22. B. J. Natalizio, S. R. Wente, Postage for the messenger: designating routes for nuclear mRNA export. Trends in cell biology 23, 365-373 (2013).
23. M. Okamura, H. Inose, S. Masuda, RNA Export through the NPC in Eukaryotes. Genes (Basel) 6, 124-149 (2015).
24. K. Thakar, S. Karaca, S. A. Port, H. Urlaub, R. H. Kehlenbach, Identification of CRM1-dependent Nuclear Export Cargos Using Quantitative Mass Spectrometry. Mol Cell Proteomics 12, 664-678 (2013).
25. C. C. Chou et al., TDP-43 pathology disrupts nuclear pore complexes and nucleocytoplasmic transport in ALS/FTD. Nature neuroscience 21, 228-239 (2018).
26. B. Eftekharzadeh et al., Tau Protein Disrupts Nucleocytoplasmic Transport in Alzheimer's Disease. Neuron 99, 925-940.e927 (2018).
27. J. C. Grima et al., Mutant Huntingtin Disrupts the Nuclear Pore Complex. Neuron 94, 93-107.e106 (2017).
28. J. V. Thevathasan et al., Nuclear pores as versatile reference standards for quantitative superresolution microscopy. Nature methods 16, 1045-1053 (2019).
29. W. Cheng et al., CRISPR-Cas9 Screens Identify the RNA Helicase DDX3X as a Repressor of C9ORF72 (GGGGCC)n Repeat-Associated Non-AUG Translation. Neuron 104, 885-898.e888 (2019).
30. B. E. Black et al., NXT1 is necessary for the terminal step of Crml-mediated nuclear export. The Journal of cell biology 152, 141-155 (2001).
31. J. McCullough, A. Frost, W. I. Sundquist, Structures, Functions, and Dynamics of ESCRT-III/Vps4 Membrane Remodeling and Fission Complexes. Annu Rev Cell Dev Biol 34, 85-109 (2018).
32. C. P. Lusk, N. R. Ader, CHMPions of repair: Emerging perspectives on sensing and repairing the nuclear envelope barrier. Current opinion in cell biology 64, 25-33 (2020).
33. G. Skibinski et al., Mutations in the endosomal ESCRTIII-complex subunit CHMP2B in frontotemporal dementia. Nature genetics 37, 806-808 (2005).
34. C. Lagier-Tourenne et al., Targeted degradation of sense and antisense C9orf72 RNA foci as therapy for ALS and frontotemporal degeneration. Proceedings of the National Academy of Sciences of the United States of America 110, E4530-4539 (2013).
35. A. D. Sennepin et al., Multiple reprobing of Western blots after inactivation of peroxidase activity by its substrate, hydrogen peroxide. Anal Biochem 393, 129-131 (2009).
36. Ling, S. C., Polymenidou, M. & Cleveland, D. W. Converging mechanisms in ALS and FTD: disrupted RNA and protein homeostasis. Neuron 79, 416-438, doi:10.1016/j.neuron.2013.07.033 (2013).
37. Taylor, J. P., Brown, R. H., Jr. & Cleveland, D. W. Decoding ALS: from genes to mechanism. Nature 539, 197-206, doi:10.1038/nature20413 (2016).
38. Kim, G., Gautier, O., Tassoni-Tsuchida, E., Ma, X. R. & Gitler, A. D. ALS Genetics: Gains, Losses, and Implications for Future Therapies. Neuron, doi:10.1016/j.neuron.2020.08.022 (2020).
39. Sances, S. et al. Modeling ALS with motor neurons derived from human induced pluripotent stem cells. Nature neuroscience 19, 542-553, doi:10.1038/nn.4273 (2016).
40. Zhang, X., Hu, D., Shang, Y. & Qi, X. Using induced pluripotent stem cell neuronal models to study neurodegenerative diseases. Biochimica et biophysica acta. Molecular basis of disease, doi:10.1016/j.bbadis.2019.03.004 (2019).
41. Donnelly, C. J. et al. RNA toxicity from the ALS/FTD C9ORF72 expansion is mitigated by antisense intervention. Neuron 80, 415-428, doi:10.1016/j.neuron.2013.10.015 (2013).
42. Gendron, T. F. et al. Poly(GP) proteins are a useful pharmacodynamic marker for C9ORF72-associated amyotrophic lateral sclerosis. Science translational medicine 9, doi:10.1126/scitranslmed.aai7866 (2017).
43. Coyne, A. N. et al. G(4)C(2) Repeat RNA Initiates a POM121-Mediated Reduction in Specific Nucleoporins in C9orf72 ALS/FTD. Neuron, doi:10.1016/j.neuron.2020.06.027 (2020).
44. Beck, M. & Hurt, E. The nuclear pore complex: understanding its function through structural insight. Nature reviews. Molecular cell biology 18, 73-89, doi:10.1038/nrm.2016.147 (2017).
45. Lin, D. H. & Hoelz, A. The Structure of the Nuclear Pore Complex (An Update). Annual review of biochemistry, doi:10.1146/annurev-biochem-062917-011901 (2019).
46. Raices, M. & D'Angelo, M. A. Nuclear pore complex composition: a new regulator of tissue-specific and developmental functions. Nature reviews. Molecular cell biology 13, 687-699, doi:10.1038/nrm3461 (2012).
47. Raices, M. & D'Angelo, M. A. Nuclear pore complexes and regulation of gene expression. Current opinion in cell biology 46, 26-32, doi:10.1016/j.ceb.2016.12.006 (2017).
48. Webster, B. M., Colombi, P., Jager, J. & Lusk, C. P. Surveillance of nuclear pore complex assembly by ESCRT-III/Vps4. Cell 159, 388-401, doi:10.1016/j.cell.2014.09.012 (2014).
49. Thaller, D. J. et al. An ESCRT-LEM protein surveillance system is poised to directly monitor the nuclear envelope and nuclear transport system. eLife 8, doi:10.7554/eLife.45284 (2019).
50. Webster, B. M. et al. Chm7 and Heh1 collaborate to link nuclear pore complex quality control with nuclear envelope sealing. The EMBO journal 35, 2447-2467, doi:10.15252/embj.201694574 (2016).
51. Toyama, B. H. et al. Visualization of long-lived proteins reveals age mosaicism within nuclei of postmitotic cells. The Journal of cell biology, doi:10.1083/jcb.201809123 (2018).
52. Lee, C. W. et al. Selective autophagy degrades nuclear pore complexes. Nature cell biology 22, 159-166, doi:10.1038/s41556-019-0459-2 (2020).
53. Tomioka, Y. et al. TORC1 inactivation stimulates autophagy of nucleoporin and nuclear pore complexes. The Journal of cell biology 219, doi:10.1083/jcb.201910063 (2020).
54. Lusk, C. P. & Ader, N. R. CHMPions of repair: Emerging perspectives on sensing and repairing the nuclear envelope barrier. Current opinion in cell biology 64, 25-33, doi:10.1016/j.ceb.2020.01.011 (2020).
55. Robberecht, W. & Philips, T. The changing scene of amyotrophic lateral sclerosis. Nature reviews. Neuroscience 14, 248-264, doi:10.1038/nrn3430 (2013).
56. Maglione, M. & Sigrist, S. J. Seeing the forest tree by tree: super-resolution light microscopy meets the neurosciences. Nature neuroscience 16, 790-797, doi:10.1038/nn.3403 (2013).
57. Schermelleh, L. et al. Subdiffraction multicolor imaging of the nuclear periphery with 3D structured illumination microscopy. Science (New York, N.Y.) 320, 1332-1336, doi:10.1126/science.1156947 (2008).
58. Clift, D. et al. A Method for the Acute and Rapid Degradation of Endogenous Proteins. Cell 171, 1692-1706.e1618, doi:10.1016/j.cell.2017.10.033 (2017).
59. Boeynaems, S., Bogaert, E., Van Damme, P. & Van Den Bosch, L. Inside out: the role of nucleocytoplasmic transport in ALS and FTLD. Acta neuropathologica 132, 159-173, doi:10.1007/s00401-016-1586-5 (2016).
60. Hutten, S. & Dormann, D. Nucleocytoplasmic transport defects in neurodegeneration Cause or consequence? Seminars in cell & developmental biology, doi:10.1016/j.semcdb.2019.05.020 (2019).
61. Jovicic, A., Paul, J. W., 3rd & Gitler, A. D. Nuclear transport dysfunction: a common theme in amyotrophic lateral sclerosis and frontotemporal dementia. Journal of neurochemistry 138 Suppl 1, 134-144, doi:10.1111/jnc.13642 (2016).
62. Kim, H. J. & Taylor, J. P. Lost in Transportation: Nucleocytoplasmic Transport Defects in ALS and Other Neurodegenerative Diseases. Neuron 96, 285-297, doi:10.1016/j.neuron.2017.07.029 (2017).
63. Gu, M. et al. LEM2 recruits CHMP7 for ESCRT-mediated nuclear envelope closure in fission yeast and human cells. Proceedings of the National Academy of Sciences of the United States of America 114, E2166-e2175, doi:10.1073/pnas.1613916114 (2017).
64. Vietri, M. et al. Unrestrained ESCRT-III drives micronuclear catastrophe and chromosome fragmentation. Nature cell biology 22, 856-867, doi:10.1038/s41556-020-0537-5 (2020).
65. Thaller, D. J. et al. Direct PA-binding by Chm7 is required for nuclear envelope surveillance at herniations. bioRxiv, 2020.2005.2004.074880, doi:10.1101/2020.05.04.074880 (2020).
66. Gao, J., Wang, L., Huntley, M. L., Perry, G. & Wang, X. Pathomechanisms of TDP-43 in neurodegeneration. Journal of neurochemistry, doi:10.1111/jnc.14327 (2018).
67. Dickmanns, A., Kehlenbach, R. H. & Fahrenkrog, B. Nuclear Pore Complexes and Nucleocytoplasmic Transport: From Structure to Function to Disease. International review of cell and molecular biology 320, 171-233, doi:10.1016/bs.ircmb.2015.07.010 (2015).
68. Coyne, A. N., Zaepfel, B. L. & Zarnescu, D. C. Failure to Deliver and Translate-New Insights into RNA Dysregulation in ALS. Frontiers in cellular neuroscience 11, 243, doi:10.3389/fnce1.2017.00243 (2017).
69. Suk, T. R. & Rousseaux, M. W. C. The role of TDP-43 mislocalization in amyotrophic lateral sclerosis. Molecular neurodegeneration 15, 45, doi:10.1186/s13024-020-00397-1 (2020).
70. Melamed, Z. et al. Premature polyadenylation-mediated loss of stathmin-2 is a hallmark of TDP-43-dependent neurodegeneration. Nature neuroscience 22, 180-190, doi:10.1038/s41593-018-0293-z (2019).
71. Prudencio, M. et al. Truncated stathmin-2 is a marker of TDP-43 pathology in frontotemporal dementia. The Journal of clinical investigation, doi:10.1172/jci139741 (2020).
72. DeTure, M. A. & Dickson, D. W. The neuropathological diagnosis of Alzheimer's disease. Molecular neurodegeneration 14, 32, doi:10.1186/s13024-019-0333-5 (2019).
73. Johnson, V. E., Stewart, W., Trojanowski, J. Q. & Smith, D. H. Acute and chronically increased immunoreactivity to phosphorylation-independent but not pathological TDP-43 after a single traumatic brain injury in humans. Acta neuropathologica 122, 715-726, doi:10.1007/s00401-011-0909-9 (2011).
74. Thammisetty, S. S. et al. Age-related deregulation of TDP-43 after stroke enhances NF-κB-mediated inflammation and neuronal damage. J Neuroinflammation 15, 312, doi:10.1186/s12974-018-1350-y (2018).
75. DeVos, S. L. & Miller, T. M. Antisense oligonucleotides: treating neurodegeneration at the level of RNA. Neurotherapeutics: the journal of the American Society for Experimental NeuroTherapeutics 10, 486-497, doi:10.1007/s13311-013-0194-5 (2013).
76. Schoch, K. M. & Miller, T. M. Antisense Oligonucleotides: Translation from Mouse Models to Human Neurodegenerative Diseases. Neuron 94, 1056-1070, doi:10.1016/j.neuron.2017.04.010 (2017).
77. Melchior, F. Ran GTPase cycle: oOne mechanism—two functions. Current biology: CB 11, R257-260 (2001).
78. Eftekharzadeh, B. et al. Tau Protein Disrupts Nucleocytoplasmic Transport in Alzheimer's Disease. Neuron 99, 925-940.e927, doi:10.1016/j.neuron.2018.07.039 (2018).
79. Grima, J. C. et al. Mutant Huntingtin Disrupts the Nuclear Pore Complex. Neuron 94, 93-107.e106, doi:10.1016/j.neuron.2017.03.023 (2017).
80. Zhang, K. et al. The C9orf72 repeat expansion disrupts nucleocytoplasmic transport. Nature 525, 56-61, doi:10.1038/nature14973 (2015).
81. Chou, C. C. et al. TDP-43 pathology disrupts nuclear pore complexes and nucleocytoplasmic transport in ALS/FTD. Nature neuroscience 21, 228-239, doi:10.1038/s41593-017-0047-3 (2018).
82. Chew, J. et al. Aberrant deposition of stress granule-resident proteins linked to C9orf72-associated TDP-43 proteinopathy. Molecular neurodegeneration 14, 9, doi:10.1186/s13024-019-0310-z (2019).
83. Zhang, Y. J. et al. Poly(GR) impairs protein translation and stress granule dynamics in C9orf72-associated frontotemporal dementia and amyotrophic lateral sclerosis. Nature medicine, doi:10.1038/s41591-018-0071-1 (2018).
84. Zhang, Y. J. et al. C9ORF72 poly(GA) aggregates sequester and impair HR23 and nucleocytoplasmic transport proteins. Nature neuroscience 19, 668-677, doi:10.1038/nn.4272 (2016).
85. Zhang, Y. J. et al. Heterochromatin anomalies and double-stranded RNA accumulation underlie C9orf72 poly(PR) toxicity. Science (New York, N.Y.) 363, doi:10.1126/science.aav2606 (2019).
86. Thevathasan, J. V. et al. Nuclear pores as versatile reference standards for quantitative superresolution microscopy. Nature methods 16, 1045-1053, doi:10.1038/s41592-019-0574-9 (2019).
87. McCullough, J., Frost, A. & Sundquist, W. I. Structures, Functions, and Dynamics of ESCRT-III/Vps4 Membrane Remodeling and Fission Complexes. Annu Rev Cell Dev Biol 34, 85-109, doi:10.1146/annurev-cellbio-100616-060600 (2018).
88. Skibinski, G. et al. Mutations in the endosomal ESCRTIII-complex subunit CHMP2B in frontotemporal dementia. Nature genetics 37, 806-808, doi:10.1038/ng1609 (2005).
89. Chang, X. L., Tan, M. S., Tan, L. & Yu, J. T. The Role of TDP-43 in Alzheimer's Disease. Molecular neurobiology 53, 3349-3359, doi:10.1007/s12035-015-9264-5 (2016).
90. Sennepin, A. D. et al. Multiple reprobing of Western blots after inactivation of peroxidase activity by its substrate, hydrogen peroxide. Anal Biochem 393, 129-131, doi:10.1016/j.ab.2009.06.004 (2009).

1. A method for screening compounds which reduce or inhibit CHMP7 in a neuronal cell or population of cells, the method comprising administering to a neuronal cell or population of cells at least one compound and determining if the compound reduces or inhibits CHMP7 expression in the cell or population of cells.

2. The method of claim 1, wherein the compound is a small molecule or a pharmaceutically acceptable salt thereof, an antibody or antigen-binding fragment thereof, an oligonucleotide, small-interfering RNA, a microRNA, a peptide, a peptidomimetic, or any combinations thereof.

3. The method of claim 2, wherein the oligonucleotide is an antisense oligonucleotide.

4. A pharmaceutical composition comprising a compound determined pursuant to the method of claim 1.

5. A method of inhibiting CHMP7 expression in a neuronal cell or population of cells, the method comprising administering to the cell or population of cells an effective amount of a CHMP7 inhibiting agent.

6. The method of claim 5, wherein the CHMP7 inhibiting agent is a small molecule or a pharmaceutically acceptable salt thereof, an antibody or antigen-binding fragment thereof, an oligonucleotide, small-interfering RNA, a microRNA, a peptide, a peptidomimetic, or any combinations thereof.

7. The method of claim 6, wherein the oligonucleotide is an antisense oligonucleotide.

8. A method of treating a neurodegenerative disease in a subject in need thereof, the method comprising administering to the subject a therapeutically effective amount of a CHMP7 inhibiting agent.

9. The method of claim 9, wherein the CHMP7 inhibiting agent is administered to a subject in an amount of about 0.001 mg/kg to about 1000 mg/kg.

10. The method of claim 8 or claim 9, wherein the subject is a human.

11. The method of any of claims 8-10, wherein the CHMP7 inhibiting agent is a small molecule or a pharmaceutically acceptable salt thereof, an antibody or antigen-binding fragment thereof, an oligonucleotide, small-interfering RNA, a microRNA, a peptide, a peptidomimetic, or any combinations thereof.

12. The method of claim 11, wherein the oligonucleotide is an antisense oligonucleotide.

13. The method of any of claims 8-12, wherein the neurodegenerative disease is Amyotrophic Lateral Sclerosis, Huntington's disease, Alzheimer's disease, Parkinson's disease, Lewy body dementia, multiple sclerosis, or frontotemporal degeneration.

14. The method of claim 13, wherein the neurodegenerative disease is Amyotrophic Lateral Sclerosis or frontotemporal degeneration.


SEQUENCE LISTING (25 sequences)

SEQ ID NO: 1 - protein, 6 aa, Artificial sequence
  Synthetic
GGGGCC

SEQ ID NO: 2 - DNA, 20 bp, Artificial sequence
  Synthetic
cctataggactatccaggaa

SEQ ID NO: 3 - DNA, 20 bp, Artificial sequence
  synthetic
gaaaacggtttccactgtat

SEQ ID NO: 4 - DNA, 20 bp, Artificial sequence
  Synthetic
tgttaccctcagataccgcc

SEQ ID NO: 5 - DNA, 22 bp, Artificial sequence
  Synthetic
tgtgacagttggaatgcagtga

SEQ ID NO: 6 - DNA, 25 bp, Artificial sequence
  Synthetic
gccacttaaagcaatctctgtcttg

SEQ ID NO: 7 - DNA, 21 bp, Artificial sequence
  Synthetic
tcgactctttgcccaccgcca

SEQ ID NO: 8 - DNA, 20 bp, Artificial sequence
  Synthetic
gggtctagcaagagcaggtg

SEQ ID NO: 9 - DNA, 21 bp, Artificial sequence
  Synthetic
gtcttggcaacagctggagat

SEQ ID NO: 10 - DNA, 24 bp, Artificial sequence
  Synthetic
tgatgtcgactctttgcccaccgc

SEQ ID NO: 11 - DNA, 26 bp, Artificial sequence
  Synthetic
agaaatgagagggaaagtaaaaatgc

SEQ ID NO: 12 - DNA, 38 bp, Artificial sequence
  Synthetic
cgactggagcacgaggacactgacggctgccgggaaga

SEQ ID NO: 13 - DNA, 23 bp, Artificial sequence
  Synthetic
aggagagcccccgcttctacccg

SEQ ID NO: 14 - DNA, 20 bp, Artificial sequence
  Synthetic
atgtgatgctattaatagga

SEQ ID NO: 15 - DNA, 20 bp, Artificial sequence
  Synthetic
agctgtccatgctgtcactg

SEQ ID NO: 16 - DNA, 20 bp, Artificial sequence
  Synthetic
ggtggcttcaagatcagctc

SEQ ID NO: 17 - DNA, 20 bp, Artificial sequence
  Synthetic
ggactcggcagaagaccttc

SEQ ID NO: 18 - DNA, 20 bp, Artificial sequence
  Synthetic
gcaggctgtctgtctctctc

SEQ ID NO: 19 - DNA, 19 bp, Artificial sequence
  Synthetic
gaaggtgaaggtcggagtc

SEQ ID NO: 20 - DNA, 20 bp, Artificial sequence
  Synthetic
gaagatggtgatgggatttc

SEQ ID NO: 21 - protein, 11 aa, Artificial sequence
  Synthetic
LEAELEKLSLS

SEQ ID NO: 22 - RNA, 19 nt, Artificial sequence
  Synthetic
cgaccuugguaaacggaaa

SEQ ID NO: 23 - RNA, 19 nt, Artificial sequence
  Synthetic
ggguuuauccugucgcuaa

SEQ ID NO: 24 - RNA, 19 nt, Artificial sequence
  Synthetic
ggagguguaucgucuguau

SEQ ID NO: 25 - RNA, 19 nt, Artificial sequence
  Synthetic
guaacaaauggcuuagauu